(19) United States
(12) Patent Application Publication
(10) Pub. No.: US 2024/0373830 A1
(43) Pub. Date: Nov. 14, 2024

(54) METHODS OF MODULATING SEX RATIO

(71) Applicant: Whitehead Institute for Biomedical Research, Cambridge, MA (US)

(72) Inventors: Jennifer Hughes, Waltham, MA (US); David C. Page, Winchester, MA (US); Helen Skaletsky, Arlington, MA (US); Peter K. Nicholls, Sowerby Bridge (GB)

(21) Appl. No.: 17/530,345

(22) Filed: Nov. 18, 2021

Related U.S. Application Data
(60) Provisional application No. 63/115,602, filed on Nov. 18, 2020.

(51) Int. Cl. A01K 67/027; C12Q 1/6879

(52) U.S. Cl. CPC A01K 67/0276; C12Q 1/6879; A01K 2217/058; A01K 2227/101; C12Q 2600/158

(57) ABSTRACT

Disclosed herein are compositions, methods, and transgenic animals for sex biasing offspring of male animals, as well as methods of screening for sex biasing agents.

### RELATED APPLICATIONS

This application claims the benefit of U.S. Provisional Application No. 63/115,602, filed Nov. 18, 2020, the entire teachings of which are incorporated herein by reference.

### STATEMENT REGARDING SEQUENCE LISTING

A Sequence Listing associated with this application is provided in text format in lieu of a paper copy and is hereby incorporated by reference into the specification. The name of the text file containing the Sequence Listing is WIBR-174-101-Sequence Listing.txt. The text file is 47.1 KB, was created on May 19, 2022, and is being submitted electronically via EFS-Web.

### BACKGROUND OF THE INVENTION

The manipulation of livestock sex is of great commercial interest because the two sexes are used for different purposes. For example, females produce milk, and males may have greater mass for meat. Many non-genetic methods to alter livestock (e.g., cattle) sex ratio exist, including sorting X- and Y-bearing sperm and sexing embryos prior to implantation. No genetic methods to change sex ratio are currently in use, although a group at UC Davis has created a bull calf designed to produce a greater number of male offspring via insertion of the male-determining gene SRY on chromosome 17 (www.ucdavis.edu/news/meet-cosmo-bull-calf-designed-produce-75-male-offspring/). However, males produced by this method will not be fertile, so the trait cannot be bred. Thus, there is still a need for methods of manipulation of livestock sex without producing offspring that are infertile.

### SUMMARY OF THE INVENTION

### Testis Specific Gene Located in a MSY Having an X-Linked Homolog

The inventors have, for the first time, obtained a high-resolution map of Bos taurus (i.e., domestic cattle) Y-chromosome, which has resisted efforts at accurate sequencing due to massive amplification of genes that cofound routine PCR based sequencing methods. Surprisingly, the inventors have found that the Y-chromosome comprises multiple copies of a testis specific gene HSFY for which a multiply copied X-linked homolog HSFX also occurs. Similar unique multiple copy x and y chromosome architecture has been previously found in mice (but not other mammals). The inventors herein disclose methods of biasing the sex of mammals, including domestic cattle, by manipulation of copy number, expression, and activity of these testis specific genes having X-linked homologs.

Some aspects of the present disclosure are directed to a method of biasing the sex ratio of offspring of a male mammal, comprising contacting the mammal with an agent that modulates the expression or activity of a testis specific gene located in a male-specific region of the Y chromosome (MSY) having an X-linked homolog, or the X-linked homolog thereof, wherein the mammal is not a mouse. In some embodiments, the mammal is not a rodent. In some embodiments the mammal is uniparous. In some embodiments, the testis specific gene has at least two copies on the MSY. In some embodiments, the testis specific gene has at least ten copies on the MSY. In some embodiments, the X-linked homolog comprises at least two copies. In some embodiments, the X-linked homolog comprises at least ten copies. In some embodiments, the testis specific gene is located in an ampliconic region.

In some embodiments of the methods disclosed throughout this disclosure, reducing the expression or activity of a testis specific gene (or deleting/inactivating one or more copies of the gene) located in a male-specific region of the Y chromosome (MSY) having an X-linked homolog increases the sex bias towards more female offspring. This may be of interest, for example, in domesticated cattle used for diary production because only cows produce milk. In some embodiments of the methods disclosed throughout this disclosure, reducing the expression or activity of the X-linked homolog gene (or deleting/inactivating one or more copies of the gene) increases the sex bias towards more male offspring. This may be of interest, for example, in domesticated cattle used for meat production because bulls/steers have more muscle than female cattle.

In some embodiments, the mammal is bovine (e.g., Bos taurus), porcine, gorilla, feline, equine, or human, or wherein the mammal is an ungulate (e.g., livestock ungulate). In some embodiments, the sex ratio is biased to males. In some embodiments, the sex ratio is biased toward females.

In some embodiments, the agent comprises an RNAi agent or antisense oligonucleotide specifically reducing or eliminating the expression of the testis specific gene or X-linked homolog thereof. In some embodiments, the agent comprises a targeting endonuclease. In some embodiments, the agent comprises a nucleic acid, protein or small molecule that modulates the activity of a gene product of the testis specific gene or X-linked homolog thereof. In some embodiments, the agent modulates a level of a gene product of the testis specific gene or X-linked homolog thereof.

In some embodiments, the testis specific gene is HSFY. In some embodiments, the X-linked homolog thereof is HSFX.

In some embodiments, the agent is locally administered to the mammal's reproductive cells.

Some aspects of the present invention are directed to a non-human mammal (e.g., Bos taurus) comprising a non-naturally occurring mutation in a testis specific gene located in a male-specific region of the Y chromosome (MSY) having an X-linked homolog, or the X-linked homolog thereof. In some embodiments, the testis specific gene has at least two copies on the MSY. In some embodiments, the testis specific gene has at least ten copies on the MSY. In some embodiments, the X-linked homolog comprises at least two copies. In some embodiments, the X-linked homolog comprises at least ten copies. In some embodiments, the testis specific gene is located in an ampliconic region.

In some embodiments, the mammal is bovine (e.g., Bos taurus), porcine (e.g., domesticated pig), gorilla, feline, equine (e.g., domesticated horse), or human, or wherein the mammal is an ungulate (e.g., livestock ungulate).

In some embodiments, the mutation reduces expression or activity of a gene product of the gene or X-linked homolog thereof. In some embodiments, the non-human mammal comprises at least 10 less functional copies of the testis specific gene or the X-linked homolog thereof than a wild-type mammal. In some embodiments, the non-human mammal comprises no functional copies of the testis specific gene or the X-linked homolog thereof.

Some aspects of the present disclosure are directed to a non-human mammal comprising a non-naturally occurring nucleotide sequence capable of expressing an RNAi agent that reduces expression of a testis specific gene located in a male-specific region of the Y chromosome (MSY) having an X-linked homolog, or the X-linked homolog thereof. In some embodiments, the RNAi agent is an siRNA or a microRNA. In some embodiments, the RNAi agent is expressed under the control of a tissue specific promoter.

Some aspects of the present disclosure are directed to a method of selective breeding of non-human mammals to bias the sex ratio of offspring, comprising providing a wild-type copy number of a testis specific gene located in a male-specific region of the Y chromosome (MSY) having an X-linked homolog, or a wild-type copy number of the X-linked homolog thereof, identifying one or more members of a population of the non-human mammals having a copy number of the testis specific gene or X-linked homolog thereof that is greater than or less than the wild-type copy number; and selectively breeding said one or more members.

Some aspects of the present disclosure are directed to a method of screening for a candidate agent that biases the sex ratio of offspring of a mammal, comprising providing a composition comprising a cell or cell free expression system expressing the product of a testis specific gene located in a male-specific region of a Y chromosome (MSY) of a mammal having an X-linked homolog, or the X-linked homolog thereof; contacting the composition with a test agent; and measuring the expression or activity of the product, wherein if the expression or activity of the product is modulated as compared to a control then the agent is identified as a candidate agent that biases the sex ratio of offspring of a mammal. In some embodiments, the gene is HSFY or HSFX (e.g., a Bos taurus HSFY or HSFX gene sequence, a Bos taurus HSFY or HSFX gene sequence as disclosed in the examples herein).

### PRSSLY

The Applicants have further discovered that an ancient, broadly conserved gene-PRSSLY (protease, serine-like Y), located on the Y chromosome in eutherian mammals-appears to be a meiotic drive factor because it is essential for equal sex ratio in mice. Specifically, it has been surprisingly found that PRSSLY knockouts produce offspring with a female-biased sex ratio. Across eutheria, PRSSLY's expression is testis-specific, and, in mouse, it is most robustly expressed in post-meiotic germ cells. PRSSLY homologs are found on the X chromosome and autosomes in more distant animals, including marsupials, monotremes, lizards, newts, and caecilians. PRSSLY appears to be the first example of a sex-linked meiotic drive element conserved for >100 million years. The identification of PRSSLY enables the possible manipulation of sex ratios in livestock, which would be of great interest, both biologically and commercially.

Some aspects of the present disclosure are directed to a method of biasing the sex ratio of offspring of a male animal, comprising contacting the animal with an agent that modulates the expression or activity of PRSSLY or a PRSSLY homolog. In some embodiments, the agent modulates the activity of a gene product of PRSSLY or a PRSSLY homolog. In some embodiments, the PRSSLY is selected from SEQ ID NO. 11 (cattle), SEQ ID NO. 12 (pig), SEQ ID NO. 13 (sheep), and SEQ ID NO. 14 (goat).

Some aspects of the present disclosure are directed to knocking out or suppressing the expression or activity of PRSSLY or a PRSSLY homolog. In some embodiments, the knocking out or suppressing the expression or activity of PRSSLY or a PRSSLY homolog results in the biasing of the sex ratio of offspring of a male animal towards female offspring. In some embodiments, the animal is a mouse or a livestock animal. In some embodiments, the PRSSLY or a PRSSLY homolog is knockout via a targeting endonuclease. In some embodiments, the PRSSLY is selected from SEQ ID NO. 11 (cattle), SEQ ID NO. 12 (pig), SEQ ID NO. 13 (sheep), and SEQ ID NO. 14 (goat).

In some embodiments, the animal is a eutherian mammal. In some embodiments, the animal is a human, chimpanzee, marmoset, mouse lemur, tree shrew, mouse, hamster, deer mouse, beaver, squirrel, damara mole rat, chinchilla, rabbit, sea otter, ferret, walrus, sea lion, monk seal, polar bear, dog, fox, horse, bovine, antelope, sheep, goat, deer, minke whale, pig, camel, flying fox, bat, elephant, wallaby, Tasmanian devil, echidna, or newt. In some embodiments, the animal is a non-human animal. In some embodiments, the animal is a livestock animal (e.g., bovine, equine, porcine, rabbit, sheep, goat, deer, camel).

In some embodiments, the sex ratio is biased to males. In some embodiments, the sex ratio is biased toward females.

In some embodiments, the agent comprises an RNAi agent or antisense oligonucleotide specifically reducing or eliminating the expression of PRSSLY or a PRSSLY homolog. In some embodiments, the agent comprises a targeting endonuclease. In some embodiments, the agent comprises a nucleic acid, protein or small molecule that modulates the activity of a gene product of PRSSLY or a PRSSLY homolog. In some embodiments, the agent modulates a level of a gene product of PRSSLY or a PRSSLY homolog. In some embodiments, the agent is locally administered to the mammal's reproductive cells.

Some aspects of the present disclosure are directed to a transgenic non-human animal exhibiting differential expression or activity of PRSSLY or a PRSSLY homolog as compared to a control non-human animal. In some embodiments, the animal comprises a non-naturally occurring mutation that modulates the expression or activity of PRSSLY or a PRSSLY homolog as compared to a control non-human animal.

In some embodiments, the animal is a eutherian mammal. In some embodiments, the animal is a chimpanzee, marmoset, mouse lemur, tree shrew, mouse, hamster, deer mouse, beaver, squirrel, damara mole rat, chinchilla, rabbit, sea otter, ferret, walrus, sea lion, monk seal, polar bear, dog, fox, horse, bovine, antelope, sheep, goat, deer, minke whale, pig, camel, flying fox, bat, elephant, wallaby, Tasmanian devil, echidna, or newt. In some embodiments, the animal is a livestock animal.

In some embodiments, the sex ratio is biased to males. In some embodiments, the sex ratio is biased toward females.

Some aspects of the present disclosure are directed to a transgenic non-human animal comprising a non-naturally occurring nucleotide sequence capable of expressing an RNAi agent that reduces expression of PRSSLY or a PRSSLY homolog as compared to a control non-human animal. In some embodiments, the RNAi agent is an siRNA or a microRNA. In some embodiments, the RNAi agent is expressed under the control of a tissue specific promoter.

Some aspects of the present disclosure are directed to a method of screening for a candidate agent that biases the sex ratio of offspring of an animal, comprising (a) providing a composition comprising a cell or cell free expression system expressing the product of PRSSLY or a PRSSLY homolog; (b) contacting the composition with a test agent; and (c) measuring the expression or activity of the product, wherein if the expression or activity of the product is modulated as compared to a control then the agent is identified as a candidate agent that biases the sex ratio of offspring of an animal.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D show the structure and ampliconic sequence content of bull, mouse, and primate Y chromosomes. (FIG. 1A) Phylogenetic relationships among five species with SHIMS-assembled MSY sequences. Branch lengths are drawn to scale to indicate species divergence times (FIGS. 1B-1D) Triangular dot plots, to scale, of DNA sequence identities within the euchromatic MSYs of bull (FIG. 1B), mouse (FIG. 1C), and primates (FIG. 1D)—human, chimpanzee, and rhesus. Each dot represents 100% intrachromosomal identity within a 200-bp window. Direct repeats appear as horizontal lines, inverted repeats as vertical lines, and palindromes as vertical lines that nearly intersect the baseline. Below plots, schematic representations of chromosomes are shown. Sequence classes are color-coded as indicated. Other: single-copy male-specific sequences that are not homologous to the X chromosome.

FIG. 2 shows intrachromosomal similarities in bull, human, and mouse MSYs. Circos plots were generated to visualize intrachromosomal DNA similarities at three different percent identity cut-offs. Bull, mouse, and human MSYs are represented, to scale, as circle diagrams; black tick mark at top of circle denotes the artificial junction between the Yp and Yq termini; color-coding corresponds to sequence class as in FIG. 1. For each MSY sequence, step-wise 50-kb segments were compared to the remaining unmasked sequence within the MSY. Lines within plots connect each 50-kb query to its top hit (only hits >10 kb in length are plotted). Line colors indicate minimum percent nucleotide identity of hits as shown.

FIGS. 3A-3B show conservation of major MSY repeats across bovine species. Two-color FISH analyses in five different Bos taurus breeds, two Bos indicus breeds, and seven wild bovine species. (FIG. 3A) Positions of CHORI-240 BAC FISH probes within sequenced bull MSY. Five different probe combinations were used. In all experiments, the red probe is the same BAC, derived from the long-arm amplicon. The green probe differs among experiments, as indicated. (FIG. 3B) Phylogenetic tree, drawn to scale, represents evolutionary relationships among Bovine species. Representative Y chromosomes observed in extended metaphase FISH images are shown for each experiment in each species. Red and green signals derive from colored FISH probes diagrammed in (FIG. 3A).

FIGS. 4A-4E show Bull MSY gene content and expression analysis. (FIG. 4A) Tabulation of ancestral and acquired protein-coding genes in bull MSY. Only intact genes, not pseudogenes, are counted in these totals. Transcription of all single-copy genes and representative members of multicopy gene families were confirmed. Copy numbers for TSPY1 and PRAMEY1 are estimates because these gene families are located within tandem arrays, for which Applicants have only partial sequence. Copy numbers were calculated by determining BAC coverage within each array, which gives an estimate of its total size. (FIG. 4B) Plot of protein-coding gene density (number of protein-coding genes per Mb) in the five sequenced MSYs: bull, mouse, human, chimpanzee, and rhesus. (FIG. 4C) Plot of percentage of MSY protein-coding genes with testis-specific expression in the five sequenced MSYs. (FIGS. 4D-E) Gene expression analysis includes RNA-seq datasets previously generated from nine adult male Bos taurus (Holstein) tissues. Expression levels for MSY genes (FIG. 4D) and their X or autosome homologs (FIG. 4E) were estimated in transcript per million (TPM) units. TPM values are plotted on a log 10 scale. Three biological replicates were analyzed for each tissue; means with standard errors are plotted.

FIGS. 5A-5B show co-amplification of testis-specific gene families on X and Y chromosomes. (FIG. 5A) Extended metaphase and interphase FISH analysis using CHORI-240 BAC probes containing HSFY (red, C0365D18) or HSFX (red, C0054K01). (FIG. 5B) Phylogenetic analysis of HSFY and HSFX amino acid sequences in mammals; chicken autosomal homolog was used as an outgroup. Branch lengths are proportional to substitution rates. Numbers at nodes indicate support from 100 bootstrap replicates. Gray bars highlight multi-copy gene families within species. For bull, only 11 of 79 nearly identical HSFY copies for which sequences were found were included in alignment; all 11 copies of HSFX for which sequence were known were included.

FIG. 6 shows consequences of X-Y recombination suppression: Two major, parallel themes of Y chromosome evolution. Summary of major events and processes in the evolution of the mammalian X and Y chromosomes from a pair of autosomes. The initial step was the Y chromosome's acquisition of the testis-determining gene, which was followed by suppression of crossing over between the X and Y chromosomes over most of their length. Lack of X-Y crossing-over resulted in a cascade of effects apparent within the sex chromosomes and genome-wide. The soma (bottom) and germline (top) influenced independent aspects of sex chromosome evolution.

FIG. 7 shows copy number variability within MSY amplicons and HSFX among 234 bulls representing four different breeds. Relative read depth mapping is shown for five MSY ampliconic regions (TSPY1 array, TSPY2-PRAME2 array, PRAME1 array, RBMY array, and long-arm amplicon), HSFX on the X chromosome, and a Y-chromosome long-arm single-copy region as a control. Each graph shows number of reads mapping to a given region normalized by number of reads mapping to 1-Mb short-arm single-copy region on the Y chromosome (for MSY amplicons) or the X chromosome (for HSFX). Each circle represents data for one animal. Data for different breeds (Holstein, Simmental, Angus, and Jersey) are grouped separately. Black horizontal line indicates mean for each breed. Multiple-testing-corrected P-values of statistically significant differences between the means are indicated as determined by Welch t-test; * P<0.05, ** P<0.01, *** P<0.001.

FIGS. 8A-8B show gene expression analysis in purified male germ cells. Analysis includes RNA-seq datasets from whole testis and purified germ cell fractions containing pachytene spermatocytes and round spermatids. Expression for MSY genes (FIG. 8A) and their X or autosome homologs (FIG. 8B) was estimated in transcript per million (TPM) units. TPM values are plotted on a log 10 scale. The analysis includes three replicates for whole testis and single samples for each purified germ cell fraction.

FIGS. 9A-9C show the sex ratio distortion in PRSSLY mutant offspring. A: Structure of mouse PRSSLY. Exons are indicated by boxes, and introns by lines; both drawn to scale. Conserved trypsin-like serine protease domains are shaded blue. Arrows indicate positions of CRISPR guide RNAs. B and C: Total number of male and female offspring in all four mutant lines vs. controls (B) and number of male and female offspring in each mutant line (C). Two-tailed p-values are from chi-squared test, comparing offspring sex numbers in mutants vs. controls. Only statistically significant p-values are shown.

FIG. 10 shows species distribution of PRSSLY homologs. At left, tree diagram shows evolutionary relationship between species. Line length is proportional to time, with scale shown at bottom. Red asterisks indicate loss or pseudogenization of PRSSLY in a given lineage. At right, status of PRSSLY and chromosomal location (if known) are indicated.

FIG. 11 shows the PRSSLY gene structure. Exons are indicated by boxes and are drawn to scale; introns are indicated by lines and are not drawn to scale. Conserved trypsin-like serine protease domains are shaded blue. Red lines indicate putative translation start sites.

FIG. 12 shows RNA-seq analysis of PRSSLY across tissues in eight eutherian mammals. Expression levels for PRSSLY were estimated in transcript per million (TPM) units. TPM values are plotted on a log 10 scale. For some tissues, multiple biological replicates were analyzed for each tissue; means with standard errors are plotted.

FIGS. 13A-13B show RNA-seq analysis of PRSSLY across development in mouse, rat, and rabbit. (A) For single-cell RNA-seq analysis in mouse, expression levels for PRSSLY are shown as reads per million mapped reads (RPM). At right, representative spermatogenic cells are shown (created with BioRender.com). (B) For bulk RNA-seq in rat and rabbit across developmental timepoints, expression levels for PRSSLY were estimated in transcript per million (TPM) units. TPM values are plotted on a log 10 scale. For some timepoints, multiple biological replicates were analyzed for each tissue; means with standard errors are plotted.

FIG. 14 shows RNA-seq analysis of PRSSLY homologs (located on X chromosome and autosomes) across tissues in marsupials, monotremes, and lizards. Expression levels for PRSSLY homologs were estimated in transcript per million (TPM) units. TPM values are plotted on a log 10 scale. For some tissues, multiple biological replicates were analyzed for each tissue; means with standard errors are plotted.

FIG. 15 illustrates syntenic relationships in anole lizard, wallaby, and human. Gene positions (blue boxes) in vicinity of autosomal PRSSLYL in anole lizard and X-linked PRSSLX in wallaby, as well as syntenic region on human X chromosome, which is missing a PRSSLY homolog, are shown. Gene positions based on genome assemblies for anole lizard chr1 (Broad AnoCar2.0/anoCar2) and human X chromosome (GRCh38/hg38). The genome assembly for wallaby does not provide sufficient X coverage, but an X-chromosome-derived BAC sequence is available (accession number CU234131) that contains PRSSLX and upstream genes. Dotted lines connect homologous genes.

FIG. 16 shows four CRISPR-induced mutations in mouse PRSSLY. Exons are indicated by boxes and are drawn to scale; introns are indicated by lines and are not drawn to scale. Conserved trypsin-like serine protease domains are shaded blue. Red asterisks indicate premature stop codons; purple asterisk indicates splice site disruption; green box indicated retroviral insertion.

FIG. 17 provides the structure of human, chimpanzee, and rhesus PRSSLY pseudogenes. Intact and active PRSSLY in mouse lemur is shown at top. Length of longest open reading frame (ORF) is indicated by black line above gene. Exons are indicated by boxes and are drawn to scale; introns are indicated by lines and are not drawn to scale. Conserved trypsin-like serine protease domains are shaded blue.

FIG. 18 provides the phylogenetic analysis of PRSSLY nucleotide sequences. Only conserved trypsin-like serine protease domains were used for alignment. Branch lengths are proportional to substitution rates. Numbers at nodes indicate support from 100 bootstrap replicates.

FIG. 19 provides confirmation of X-linkage of PRSSLY in marsupials. PRSSLY homologs map to X-chromosome contigs in both the wallaby and Tasmanian devil genome assemblies. However, Applicants wanted to confirm X-linkage using an independent method, so Applicants performed read mapping depth coverage analysis. Separately, for wallaby and Tasmanian devil, Applicants mapped genomic reads derived from male and female animals to the female reference genome using Bowtie version 2.3.4.1 and counted the number of reads mapping to PRSSLY-containing contigs, normalized by the total number of reads mapped to the genome. The number of hits in the male (XY) genome is approximately half that in female (XX), confirming X linkage.

FIG. 20 shows sequence conservation across PRSSLY gene sequences. Dot plot analysis of deer gene sequence (which has the longest open reading frame of all known PRSSLY homologs) vs. bull, pig, mouse lemur, ferret, mouse, and tree shrew. Deer is most closely related to bull and pig, which is reflected in relatively high conservation across entire gene. In comparison, conservation is mostly limited to the conserved trypsin-like serine protease domain (blue) in mouse lemur, ferret, rat, and tree shrew. Window size=30, min. % score=60, hash value=8.

FIG. 21 shows expression of human PRSS homologs across tissues. Heat map was generated using Morpheus (software.broadinstitute.org/morpheus). Only PRSS homologs with maximum tpm greater than 1 are shown. Expression levels of PRSS33, PRSS48, and PRSS57 were below 1 tpm in all tissues examined.

FIGS. 22A-22B show gene expression analysis of PRSSLY in purified male germ cells and germ-cell-depleted testis. Analysis includes RNA-seq datasets from whole testis and purified germ cell fractions containing pachytene spermatocytes and round spermatids for bull (A) and mouse (B). For mouse, analysis includes datasets derived from embryonic day 12.5 and adult wild-type (WT) and germ-cell-depleted (W/Wv) mice. Expression was estimated in transcript per million (TPM) units. TPM values are plotted on a log 10 scale.

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, NJ, 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### Methods and Compositions for Sex Biasing by Modulating a Testis Specific Gene Located in a Male-Specific Region of the Y Chromosome (MSY) Having an X-Linked Homolog, or the X-Linked Homolog Thereof

Some aspects of the present disclosure are directed to a method of biasing the sex ratio of offspring of a male mammal, comprising contacting the mammal with an agent that modulates the expression or activity of a testis specific gene located in a male-specific region of the Y chromosome (MSY) having an X-linked homolog, or the X-linked homolog thereof.

In some embodiments, the sex ratio is biased towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more males: females offspring. In some embodiments, the sex ratio is biased towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more females: males offspring.

In some embodiments, the agent reduces the expression or activity (e.g., activity of the gen product) of the testis specific gene or the X-linked homolog thereof by at least about 10%, 20%, 30%, 40%, 50%, 60%, 70%, 80%, 90%, or more. In some embodiments, the agent increases the expression or activity (e.g., activity of the gen product) of the testis specific gene or the X-linked homolog thereof by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

The mammal is not limited. In some embodiments, the mammal is not a mouse. In some embodiments, the mammal is an ungulate. In some embodiments, the ungulate is a livestock animal. In some embodiments, the mammal is bovine, porcine, gorilla, feline, equine, primate, or human. In some embodiments, the male mammal is a bull (i.e., Bos taurus). In some embodiments, the male mammal is a human. In some embodiments, the human male has, or is at risk of developing, an X-linked recessive disease or disorder.

The testis specific gene located in the MSY is not limited as long as an X-linked homolog of the gene is also present in the genome of the mammal. In some embodiments, the testis specific gene is HSFY or VCY. In some embodiments, the X-linked homolog is HSFX or VCX. In some embodiments, the HSFY gene is accession number NM_033108, NM_153716, FJ527015, NM_001077006, NM_001040123, or GQ253469. In some embodiments, the HSFX gene is accession number NM_016153, XM_005594782, XM_001089561, XM_416447, NM_001164415, NM_001323079, NM_001351114, or GQ253474. In some embodiments, the HSFX gene is shown in SEQ ID NO: 1. In some embodiments, the HSFX gene is a Bos Taurus gene shown in SEQ ID NOS: 2-10.

In some embodiments, the testis specific gene has at least 2, 3, 4, 5, 6, 7, 8, 9, 10, 11, 12, 13, 14, 15, 20, 25, 30, 35, 40, 50, 60, 70, 75, 80, 90, 100 or more copies on the MSY. In some embodiments, the testis specific gene has at least 2 copies on the MSY. In some embodiments, the testis specific gene has at least 10 copies on the MSY.

In some embodiments, the X-linked homolog gene has at least 2, 3, 4, 5, 6, 7, 8, 9, 10, 11, 12, 13, 14, 15, 20, 25, 30, 35, 40, 50, 60, 70, 75, 80, 90, 100 or more copies on the X-chromosome. In some embodiments, the X-linked homolog gene has at least 2 copies on the X-chromosome. In some embodiments, the X-linked homolog gene has at least 10 copies on the X-chromosome.

In some embodiments, the testis specific gene is located in an ampliconic region. As used herein, an ampliconic region comprises ampliconic sequences, which are euchromatic repeats that display >99% identity over more than 10 kb. In some embodiments, the testis specific gene is located in an MSY comprising at least about 30%, 40%, 50%, 60%, 70%, 80%, 90%, 95%, 99%, or 99.5% ampliconic sequences.

The agent is not limited and may be any suitable agent capable of modulating the expression or activity of a testis specific gene located in a male-specific region of the Y chromosome (MSY) having an X-linked homolog, or the X-linked homolog thereof. The agent may be any agent described herein.

In some embodiments, the agent is an antagonist of a gene product of the testis specific gene or X-linked homolog thereof. In some embodiments, the agent is an agonist of a gene product of the testis specific gene or X-linked homolog thereof. In some embodiments, the agent is encoded by a synthetic RNA (e.g., modified mRNAs). In some embodiments, the synthetic agent expresses a gene product of the testis specific gene or X-linked homolog thereof.

In some embodiments, the agent is a targetable nuclease and, if appropriate, a guide molecule (e.g., one or more gRNA). In some embodiments, the agent is capable of making a mutation in a of a gene product of the testis specific gene or X-linked homolog thereof as described herein.

In some embodiments, the agent is a targetable nuclease capable of introducing a mutation reducing the expression or activity of a gene product of the testis specific gene or X-linked homolog thereof, or increasing the expression or activity of a gene product of the testis specific gene or X-linked homolog thereof. In some embodiments, the agent is an RNA-guided nucleases (RGNs) (e.g., the Cas proteins of the CRISPR/Cas Type II system targetable nuclease) and RNA template (e.g., gRNA) capable of specifically introducing a mutation reducing the expression or activity of a gene product of the testis specific gene or X-linked homolog thereof, or increasing the expression or activity of a gene product of the testis specific gene or X-linked homolog thereof.

In some embodiments, the agent comprises an “RNA interference” (RNAi) agent specifically reducing or eliminating the expression of the testis specific gene or X-linked homolog thereof. In some embodiments, the agent comprises antisense oligonucleotide specifically reducing or eliminating the expression of the testis specific gene or X-linked homolog thereof.

In some embodiments, an oligonucleotide and the target RNA sequence (e.g., an mRNA of a testis specific gene located in a male-specific region of the Y chromosome (MSY) having an X-linked homolog, or the X-linked homolog thereof) have 100% sequence complementarity. In some aspects, an oligonucleotide may comprise sequence variations, e.g., insertions, deletions, and single point mutations, relative to the target sequence. In some embodiments, an oligonucleotide has at least 70% sequence identity or complementarity to the target RNA (e.g., mRNA, pre-mRNA, or nascent RNA of a testis specific gene located in a male-specific region of the Y chromosome (MSY) having an X-linked homolog, or the X-linked homolog thereof). In certain embodiments, an oligonucleotide has at least 70%, 75%, 80%, 85%, 90%, 95%, 97%, 99%, or 100% sequence identity to the target sequence.

Some aspects of the present disclosure are directed to compositions (e.g., pharmaceutical compositions including compositions for veterinary use) comprising an agent as described herein for sex biasing of offspring of a male mammal.

### Transgenic Animals Comprising a Non-Naturally Occurring Mutation in a Testis Specific Gene Located in a Male-Specific Region of the Y Chromosome (MSY) Having an X-Linked Homolog, or the X-Linked Homolog Thereof

Some aspects of the present invention are directed to a non-human mammal (i.e., transgenic non-human mammal) comprising a non-naturally occurring mutation in a testis specific gene located in a male-specific region of the Y chromosome (MSY) having an X-linked homolog, or the X-linked homolog thereof. The non-human mammal may be any suitable mammal described herein. In some embodiments, the mammal is a bull (Bos Taurus), bovine or other ungulate species (e.g., livestock ungulate). The testis specific gene or X-linked homolog thereof may be any testis specific gene or X-linked homolog thereof described herein. In some embodiments, the testis specific gene or X-linked homolog thereof is a Bos taurus HSFY or HSFX gene (e.g., a Bos taurus gene described or disclosed herein). In some embodiments, the testis specific gene has at least 2, 5, 10, 20, 30, 50, or more copies on the MSY. In some embodiments, the X-linked homolog gene has at least 2, 5, 10, 20, 30, 50, or more copies on the X-chromosome. In some embodiments, the testis specific gene is located in an ampliconic region.

In some embodiments, the mutation reduces expression or activity of a gene product of the gene or X-linked homolog thereof as compared to the wild-type of the non-human mammal. In some embodiments, the mutation reduces the expression or activity of the testis specific gene or the X-linked homolog thereof by at least about 10%, 20%, 30%, 40%, 50%, 60%, 70%, 80%, 90%, or more. In some embodiments, the mutation increases the expression or activity of the testis specific gene or the X-linked homolog thereof by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the sex ratio is biased by the mutation towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more males: females offspring. In some embodiments, the sex ratio is biased by the mutation towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more females: males offspring. In some embodiments, sex ratio biasing as compared to wild-type is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold towards males or females.

In some embodiments, the mutation is one or more nucleotide insertions, deletions and/or substitutions. In some embodiments, the mutation reduces or eliminates expression or activity of the gene product (e.g., a frameshift mutation, a mutation rendering the gene product non-functional (inactive) or partially functional (less active), a mutation in a regulatory region that reduces or eliminates expression, a mutation deleting all or a portion of the gene product, a mutation deleting one or more copies of a gene). In some embodiments, the mutation increases expression or activity of the gene product (e.g., a mutation enhancing gene product activity, a mutation enhancing transcription of the gene product, a mutation adding one or more additional copies of the gene).

In some embodiments, the transgenic non-human mammal comprises at least 2, 3, 4, 5, 6, 7, 8, 9, 10, 11, 12, 13, 14, 15, 20, 25, 30, 35, 40, 50, 60, 70, 75, 80, 90, or 100 less functional copies of the testis specific gene or the X-linked homolog thereof than a wild-type mammal. In some embodiments, the transgenic non-human mammal comprises at least 10 less functional copies of the testis specific gene or the X-linked homolog thereof than a wild-type mammal. In some embodiments, the transgenic non-human mammal comprises at least 1 less functional copy of the testis specific gene or the X-linked homolog thereof than a wild-type mammal. In some embodiments, the transgenic non-human mammal comprises no functional copies of the testis specific gene or the X-linked homolog thereof.

In some embodiments, the transgenic non-human mammal comprises at least 2, 3, 4, 5, 6, 7, 8, 9, 10, 11, 12, 13, 14, 15, 20, 25, 30, 35, 40, 50, 60, 70, 75, 80, 90, or 100 more copies of the testis specific gene or the X-linked homolog thereof than a wild-type mammal. In some embodiments, the transgenic non-human mammal comprises at least 10 more copies of the testis specific gene or the X-linked homolog thereof than a wild-type mammal. In some embodiments, the transgenic non-human mammal comprises at least 1 more copy of the testis specific gene or the X-linked homolog thereof than a wild-type mammal.

Some aspects of the present disclosure are related to a non-human mammal (i.e., a transgenic mammal) comprising a non-naturally occurring nucleotide sequence capable of expressing an RNAi agent as described herein that reduces expression of a testis specific gene located in a male-specific region of the Y chromosome (MSY) having an X-linked homolog, or the X-linked homolog thereof. In some embodiments, the RNAi agent is an siRNA, shRNA, or a microRNA.

In some embodiments, the RNAi agent is expressed under the control of a tissue specific promoter. The tissue specific promoter is not limited and may be any suitable promoter. In some embodiments, the promoter is testis specific (e.g., a bull testis specific promoter). In some embodiments, the bull testis specific promoter is HSPB9, ADAM18, or RELN. In some embodiments, the promoter is an inducible promoter or tissue specific inducible promoter. In some embodiments, the transgenic gene is integrated into a safe harbor locus.

Standard methods of generating genetically modified animals, e.g., transgenic animals that comprises exogenous genes or animals that have an alteration to an endogenous gene, e.g., an insertion or an at least partial deletion or replacement can be used. In some embodiments, the genetically modified animal having a transgene is produced by a method comprising DNA microinjection, embryonic stem cell-mediated gene transfer, somatic cell nuclear transfer, retrovirus-mediated gene transfer, transposon-mediated gene transfer, or sperm-mediated gene transfer. In some embodiments, the transgenic animal is produced by a method comprising contacting an embryonic stem cell, zygote, or embryo with an agent as described herein wherein the agent is a targetable nuclease and, if appropriate, a guide molecule (e.g., one or more gRNA).

### Breeding Methods for Modulating the Copy Number of the Testis Specific Gene or X-Linked Homolog Thereof

Some aspects of the present disclosure are directed to a method of selective breeding of non-human mammals to bias the sex ratio of offspring, comprising providing a wild-type copy number of a testis specific gene located in a male-specific region of the Y chromosome (MSY) having an X-linked homolog, or a wild-type copy number of the X-linked homolog thereof, identifying one or more members of a population of the non-human mammals having a copy number of the testis specific gene or X-linked homolog thereof that is greater than or less than the wild-type copy number, and selectively breeding said one or more members. In some embodiments, the selective breeding increases the copy number of the testis specific gene or X-linked homolog thereof. In some embodiments, the selective breeding decreases the copy number of the testis specific gene or X-linked homolog thereof.

In some embodiments, the offspring of the one or more members are screened for the presence of more or less copies of the testis specific gene or X-linked homolog thereof and selectively bred. This method may be repeated one or more times until a stable line of non-human animals is obtained having a biased sex ratio as compared to the wild-type animal.

The non-human mammals may be any mammals disclosed herein. In some embodiments, the mammals are cattle. In some embodiments, the mammals are livestock ungulates.

Methods of detecting the copy number of a gene in the genome of an animal are known in the art and any suitable method may be used. Said methods include, without limitation, in situ hybridization (ISH) (such as fluorescence in situ hybridization (FISH), chromogenic in situ hybridization (CISH) or silver in situ hybridization (SISH)), genomic comparative hybridization or polymerase chain reaction (such as real time quantitative PCR). In some embodiments, the method of detecting the copy number is disclosed in the Examples herein. In some embodiments, the method is or comprises Single-Haplotype Iterative Mapping and Sequencing (SHIMS) methodology (SHIMS). See, Bellott et al., Nat Protoc (2018) 13:787-809.

### Methods of Screening for Agents Modulating the Product of a Testis Specific Gene Located in a Male-Specific Region of a Y Chromosome (MSY) of a Mammal Having an X-Linked Homolog, or the X-Linked Homolog Thereof

Some aspects of the present disclosure are directed to a method of screening for a candidate agent that biases the sex ratio of offspring of a mammal, comprising providing a composition comprising a cell or cell free expression system expressing the product of a testis specific gene located in a male-specific region of a Y chromosome (MSY) of a mammal having an X-linked homolog, or the X-linked homolog thereof; contacting the composition with a test agent; and measuring the expression or activity of the product, wherein if the expression or activity of the product is modulated as compared to a control then the agent is identified as a candidate agent that biases the sex ratio of offspring of a mammal. The testis specific gene or X-linked homolog thereof is not limited and may be any one disclosed herein. In some embodiments, the gene is HSFY or HSFX. The mammal may be any mammal disclosed herein. In some embodiments, the mammal is a bull. In some embodiments, the mammal is a livestock ungulate.

Some aspects of the present disclosure are directed to a method of screening for a candidate agent that biases the sex ratio of offspring of a bull, comprising providing a composition comprising a gene product of a HSFY or HSFX gene (e.g., a Bos taurus HSFY or HSFX gene, a Bos taurus HSFY or HSFX gene disclosed herein) and cognate heat shock binding elements nucleotide sequence; contacting the composition with a test agent; and determining if the test agent reduces or eliminates binding of the gene product to the binding elements. In some embodiments, the binding of the gene product to the binding elements causes expression of a gene product and reduced expression of the gene product can be used to detect if the agent is inhibiting binding of the gene product to the binding element.

### Methods and Compositions for Sex Biasing by Modulating PRSSLY or a Homolog Thereof

Some aspects of the present disclosure are directed to a method of biasing the sex ratio of offspring of a male animal, comprising contacting the animal with an agent that modulates the expression or activity of PRSSLY or a PRSSLY homolog (e.g., GenBank Accession Number KJ780361 or a homolog thereof). In some embodiments, the agent modulates the activity of a gene product of PRSSLY or a PRSSLY homolog.

In some embodiments, the sex ratio is biased to males. In some embodiments, the sex ratio is biased toward females. In some embodiments, the sex ratio is biased towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more males: females offspring. In some embodiments, the sex ratio is biased towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more females: males offspring.

In some embodiments, the agent reduces the expression or activity (e.g., activity of the gene product) of PRSSLY or a PRSSLY homolog by at least about 10%, 20%, 30%, 40%, 50%, 60%, 70%, 80%, 90%, or more. In some embodiments, the agent increases the expression or activity (e.g., activity of the gene product) of PRSSLY or a PRSSLY homolog by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

The animal is not limited. In some embodiments, the wild-type animal expresses PRSSLY or a PRSSLY homolog. In some embodiments, the wild-type animal expresses PRSSLY. In some embodiments, the PRSSLY is selected from SEQ ID NO. 11 (cattle), SEQ ID NO. 12 (pig), SEQ ID NO. 13 (sheep), and SEQ ID NO. 14 (goat). In some embodiments, the animal is a eutherian mammal. In some embodiments, the animal is a human, chimpanzee, marmoset, mouse lemur, tree shrew, mouse, hamster, deer mouse, beaver, squirrel, damara mole rat, chinchilla, rabbit, sea otter, ferret, walrus, sea lion, monk seal, polar bear, dog, fox, horse, bovine, antelope, sheep, goat, deer, minke whale, pig, camel, flying fox, bat, elephant, wallaby, Tasmanian devil, echidna, or newt. In some embodiments, the animal is a non-human animal. In some embodiments, the animal is a livestock animal (e.g., bovine, equine, porcine, rabbit, sheep, goat, deer, camel). In some embodiments, the animal is a pig. In some embodiments, the animal is a bull. In some embodiments, the animal is a dog. In some embodiments, the animal is a mouse or rat.

The agent is not limited and may be any suitable agent capable of modulating the expression or activity of PRSSLY or a PRSSLY homolog. The agent may be any agent described herein.

In some embodiments, the agent comprises an RNAi agent or antisense oligonucleotide specifically reducing or eliminating the expression of PRSSLY or a PRSSLY homolog. In some embodiments, the agent comprises a targeting endonuclease. In some embodiments, the agent comprises a nucleic acid, protein or small molecule that modulates the activity (e.g., increases or decreases) of a gene product of PRSSLY or a PRSSLY homolog.

In some embodiments, the agent modulates a level (e.g., increases or decreases) of a gene product of PRSSLY or a PRSSLY homolog.

In some embodiments, the agent is an antagonist of a gene product of PRSSLY or a PRSSLY homolog. In some embodiments, the agent is an agonist of a gene product of PRSSLY or a PRSSLY homolog. In some embodiments, the agent is encoded by a synthetic RNA (e.g., modified mRNAs). In some embodiments, the synthetic agent expresses a gene product of PRSSLY or a PRSSLY homolog.

In some embodiments, the agent is a targetable nuclease and, if appropriate, a guide molecule (e.g., one or more gRNA). In some embodiments, the agent is capable of making a mutation in a of a gene product of PRSSLY or a PRSSLY homolog as described herein.

In some embodiments, the agent is a targetable nuclease capable of introducing a mutation reducing the expression or activity of a gene product of PRSSLY or a PRSSLY homolog, or increasing the expression or activity of PRSSLY or a PRSSLY homolog. In some embodiments, the agent is an RNA-guided nucleases (RGNs) (e.g., the Cas proteins of the CRISPR/Cas Type II system targetable nuclease) and RNA template (e.g., gRNA) capable of specifically introducing a mutation reducing the expression or activity of a gene product of PRSSLY or a PRSSLY homolog, or increasing the expression or activity of a gene product of PRSSLY or a PRSSLY homolog.

In some embodiments, the agent comprises an “RNA interference” (RNAi) agent specifically reducing or eliminating the expression of PRSSLY or a PRSSLY homolog. In some embodiments, the agent comprises antisense oligonucleotide specifically reducing or eliminating the expression of PRSSLY or a PRSSLY homolog.

In some embodiments, an oligonucleotide and the target RNA sequence (e.g., an mRNA of PRSSLY or a PRSSLY homolog) have 100% sequence complementarity. In some aspects, an oligonucleotide may comprise sequence variations, e.g., insertions, deletions, and single point mutations, relative to the target sequence. In some embodiments, an oligonucleotide has at least 70% sequence identity or complementarity to the target RNA (e.g., mRNA, pre-mRNA, or nascent RNA of PRSSLY or a PRSSLY homolog). In certain embodiments, an oligonucleotide has at least 70%, 75%, 80%, 85%, 90%, 95%, 97%, 99%, or 100% sequence identity to the target sequence. In some embodiments, the PRSSLY is selected from SEQ ID NO. 11 (cattle), SEQ ID NO. 12 (pig), SEQ ID NO. 13 (sheep), and SEQ ID NO. 14 (goat).

Some aspects of the present disclosure are directed to compositions (e.g., pharmaceutical compositions including compositions for veterinary use) comprising an agent as described herein for sex biasing of offspring of a male animal. In some embodiments, the agent/composition is locally administered to the mammal's reproductive cells.

### Transgenic Animals Comprising a Non-Naturally Occurring Mutation in PRSSLY or a PRSSLY Homolog

Some aspects of the present disclosure are directed to a transgenic non-human animal exhibiting differential expression or activity (e.g., increased or decreased) of PRSSLY or a PRSSLY homolog as compared to a control non-human animal. In some embodiments, the animal comprises a non-naturally occurring mutation that modulates (e.g., increases or decreases) the expression or activity of PRSSLY or a PRSSLY homolog as compared to a control non-human animal.

In some embodiments, the animal is a eutherian mammal. In some embodiments, the animal is a chimpanzee, marmoset, mouse lemur, tree shrew, mouse, hamster, deer mouse, beaver, squirrel, damara mole rat, chinchilla, rabbit, sea otter, ferret, walrus, sea lion, monk seal, polar bear, dog, fox, horse, bovine, antelope, sheep, goat, deer, minke whale, pig, camel, flying fox, bat, elephant, wallaby, Tasmanian devil, echidna, or newt. In some embodiments, the animal is a livestock animal.

In some embodiments, the sex ratio is biased to males. In some embodiments, the sex ratio is biased toward females.

In some embodiments, the transgenic non-human animal has at least 2, 5, 10, 20, 30, 50, or more copies of PRSSLY or a PRSSLY homolog as compared to a control wild-type animal.

In some embodiments, the mutation reduces expression or activity of a gene product of PRSSLY or a PRSSLY homolog as compared to the wild-type of a control non-human animal. In some embodiments, the mutation reduces the expression or activity of PRSSLY or a PRSSLY homolog by at least about 10%, 20%, 30%, 40%, 50%, 60%, 70%, 80%, 90%, or more. In some embodiments, the mutation increases the expression or activity of PRSSLY or a PRSSLY homolog by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, the PRSSLY is selected from SEQ ID NO. 11 (cattle), SEQ ID NO. 12 (pig), SEQ ID NO. 13 (sheep), and SEQ ID NO. 14 (goat).

In some embodiments, the sex ratio is biased by the mutation towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more males: females offspring. In some embodiments, the sex ratio is biased by the mutation towards at least about 1:1.1, 1:1.2, 1:1.3, 1:1.4, 1:1.5, 1:1.6, 1:1.7, 1:1.8, 1:1.9, 1:2, 1:2.5, 1:3, 1:3.5; 1:4, 1:4.5, 1:5 or more females: males offspring. In some embodiments, sex ratio biasing as compared to wild-type is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold towards males or females.

In some embodiments, the mutation is one or more nucleotide insertions, deletions and/or substitutions. In some embodiments, the mutation reduces or eliminates expression or activity of the gene product (e.g., a frameshift mutation, a mutation rendering the gene product non-functional (inactive) or partially functional (less active), a mutation in a regulatory region that reduces or eliminates expression, a mutation deleting all or a portion of the gene product, a mutation deleting one or more copies of a gene). In some embodiments, the mutation increases expression or activity of the gene product (e.g., a mutation enhancing gene product activity, a mutation enhancing transcription of the gene product, a mutation adding one or more additional copies of the gene). In some embodiments, the mutation modulates (e.g., increases or decreases) PRSSLY or a PRSSLY homolog promoter activity. In some embodiments, the PRSSLY is selected from SEQ ID NO. 11 (cattle), SEQ ID NO. 12 (pig), SEQ ID NO. 13 (sheep), and SEQ ID NO. 14 (goat).

Some aspects of the present disclosure are directed to a transgenic non-human animal comprising a non-naturally occurring nucleotide sequence capable of expressing an RNAi agent that reduces expression of PRSSLY or a PRSSLY homolog as compared to a control non-human animal. In some embodiments, the RNAi agent is an siRNA or a microRNA. In some embodiments, the RNAi agent is an siRNA, shRNA, or a microRNA.

In some embodiments, the RNAi agent is expressed under the control of a tissue specific promoter. The tissue specific promoter is not limited and may be any suitable promoter. In some embodiments, the promoter is testis specific (e.g., a bull testis specific promoter). In some embodiments, the promoter is an inducible promoter or tissue specific inducible promoter. In some embodiments, the transgenic gene is integrated into a safe harbor locus.

Standard methods of generating genetically modified animals, e.g., transgenic animals that comprises exogenous genes or animals that have an alteration to an endogenous gene, e.g., an insertion or an at least partial deletion or replacement can be used. In some embodiments, the genetically modified animal having a transgene is produced by a method comprising DNA microinjection, embryonic stem cell-mediated gene transfer, somatic cell nuclear transfer, retrovirus-mediated gene transfer, transposon-mediated gene transfer, or sperm-mediated gene transfer. In some embodiments, the transgenic animal is produced by a method comprising contacting an embryonic stem cell, zygote, or embryo with an agent as described herein wherein the agent is a targetable nuclease and, if appropriate, a guide molecule (e.g., one or more gRNA).

### Breeding Methods for Modulating PRSSLY or a PRSSLY Homolog Expression or Activity

Some aspects of the present disclosure are directed to a method of selective breeding of non-human animals (e.g., non-transgenic) to bias the sex ratio of offspring, comprising identifying one or more members of a population of the non-human animals having differential (e.g., increased or decreased) PRSSLY or a PRSSLY homolog expression or activity, and selectively breeding said one or more members. In some embodiments, the selective breeding increases PRSSLY or a PRSSLY homolog expression or activity. In some embodiments, the selective breeding decreases PRSSLY or a PRSSLY homolog expression or activity. In some embodiments, the PRSSLY is selected from SEQ ID NO. 11 (cattle), SEQ ID NO. 12 (pig), SEQ ID NO. 13 (sheep), and SEQ ID NO. 14 (goat).

In some embodiments, the offspring of the one or more members are screened for modulated PRSSLY or a PRSSLY homolog expression or activity and selectively bred. This method may be repeated one or more times until a stable line of non-human animals is obtained having a biased sex ratio as compared to the wild-type animal.

The non-human animal may be any animal disclosed herein. In some embodiments, the non-human animal is a livestock animal. In some embodiments, the non-human animal is a bull or pig.

### Methods of Screening for Agents Modulating the Product of PRSSLY or a PRSSLY Homolog

Some aspects of the present disclosure are directed to a method of screening for a candidate agent that biases the sex ratio of offspring of an animal, comprising (a) providing a composition comprising a cell or cell free expression system expressing the product of PRSSLY or a PRSSLY homolog; (b) contacting the composition with a test agent; and (c) measuring the expression or activity of the product, wherein if the expression or activity of the product is modulated as compared to a control then the agent is identified as a candidate agent that biases the sex ratio of offspring of an animal. In some embodiments, the PRSSLY is selected from SEQ ID NO. 11 (cattle), SEQ ID NO. 12 (pig), SEQ ID NO. 13 (sheep), and SEQ ID NO. 14 (goat).

The animal may be any animal disclosed herein. In some embodiments, the animal is a livestock animal. In some embodiments, the animal is a bull or pig.

### Some Definitions and Embodiments

The terms “decrease”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

“Agent” is used herein to refer to any substance, compound (e.g., molecule), supramolecular complex, material, or combination or mixture thereof. In some aspects, an agent can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, peptide mimetics, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. Unless otherwise specified, the term “analog” as used herein refers to a structural analog. A structural analog of an agent may have substantially similar physical, chemical, biological, and/or pharmacological propert (ies) as the agent or may differ in at least one physical, chemical, biological, or pharmacological property. In some embodiments at least one such property differs in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, ST (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929, 226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long. In some embodiments, the nucleic acid agent codes for a gene product of the testis specific gene or X-linked homolog thereof.

“Nucleic acid construct” refers to a nucleic acid that is generated by man and is not identical to nucleic acids that occur in nature, i.e., it differs in sequence from naturally occurring nucleic acid molecules and/or comprises a modification that distinguishes it from nucleic acids found in nature. A nucleic acid construct may comprise two or more nucleic acids that are identical to nucleic acids found in nature, or portions thereof, but are not found as part of a single nucleic acid in nature.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent is an antagonist of a gene product of gene disclosed herein. In some embodiments, the agent is an agonist of a gene product disclosed here. In some embodiments, the agent is encoded by a synthetic RNA (e.g., modified mRNAs). In some embodiments, the synthetic agent expresses a gene product of a gene disclosed herein.

The synthetic RNA can encode any suitable agent described herein. Synthetic RNAs, including modified RNAs are taught in WO 2017075406, which is herein incorporated by reference. In some embodiments, the agent is, or is encoded by, a synthetic RNA (e.g., modified mRNAs) conjugated to non-nucleic acid molecules. In some embodiments, the synthetic RNAs are conjugated to (or otherwise physically associated with) a moiety that promotes cellular uptake, nuclear entry, and/or nuclear retention (e.g., peptide transport moieties or the nucleic acids). In some embodiments, the synthetic RNA is conjugated to a peptide transporter moiety, for example a cell-penetrating peptide transport moiety, which is effective to enhance transport of the oligomer into cells.

In some embodiments, the agent is a targetable nuclease and, if appropriate, a guide molecule (e.g., one or more gRNA).

The term “targetable nuclease” refers to a nuclease that can be programmed to produce site-specific DNA breaks, e.g., double-stranded breaks (DSBs), at a selected site in DNA. Such a site may be referred to as a “target site”. The target site can be selected by appropriate design of the targetable nuclease or by providing a guide molecule (e.g., a guide RNA) directs the nuclease to the target site. Examples of targetable nucleases include zinc finger nucleases (ZFNs), transcription activator-like effector nucleases (TALENs), and RNA-guided nucleases (RGNs) such as the Cas proteins of the CRISPR/Cas Type II system, and engineered meganucleases.

In some embodiments, the agent comprises an “RNA interference” (RNAi) agent.

The term “RNA interference” (RNAi) encompasses processes in which a molecular complex known as an RNA-induced silencing complex (RISC) reduces gene expression in a sequence-specific manner in, e.g., eukaryotic cells, e.g., vertebrate cells, or in an appropriate in vitro system. RISC may incorporate a short nucleic acid strand (e.g., about 16-about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA.” A guide strand may initially become associated with RISC components (in a complex sometimes termed the RISC loading complex) as part of a short double-stranded RNA (dsRNA), e.g., a short interfering RNA (siRNA). The other strand of the short dsRNA may be referred to as a “passenger strand” or “sense strand”. The complementarity of the structure formed by hybridization of a target RNA and the guide strand may be such that the strand can (i) guide cleavage of the target RNA in the RNA-induced silencing complex (RISC) and/or (ii) cause translational repression of the target RNA. Reduction of expression due to RNAi may be essentially complete (e.g., the amount of a gene product is reduced to background levels) or may be less than complete in various embodiments. For example, mRNA and/or protein level may be reduced by 50%, 60%, 70%, 75%, 80%, 85%, 90%, 95%, or more, in various embodiments. As known in the art, the complementarity between the guide strand and a target RNA need not be perfect (100%) but need only be sufficient to result in inhibition of gene expression. For example, in some embodiments 1, 2, 3, 4, 5, or more nucleotides of a guide strand may not be matched to a target RNA. “Not matched” or “unmatched” refers to a nucleotide that is mismatched (not complementary to the nucleotide located opposite it in a duplex, i.e., wherein Watson-Crick base pairing does not take place) or forms at least part of a bulge. Examples of mismatches include, without limitation, an A opposite a G or A, a C opposite an A or C, a U opposite a Cor U, a G opposite a G. A bulge refers to a sequence of one or more nucleotides in a strand within a generally duplex region that are not located opposite to nucleotide(s) in the other strand. “Partly complementary” refers to less than perfect complementarity. In some embodiments a guide strand has at least about 80%, 85%, or 90%, e.g., at least about 91%, 92%, 93%, 94%, 95%, 96%, 97%, 98%, 99%, or 100% sequence complementarity to a target RNA over a continuous stretch of at least about 15 nt, e.g., between 15 nt and 30 nt, between 17 nt and 29 nt, between 18 nt and 25 nt, between 19 nt and 23 nt, of the target RNA. In some embodiments at least the seed region of a guide strand (the nucleotides in positions 2-7 or 2-8 of the guide strand) is perfectly complementary to a target RNA. In some embodiments, a guide strand and a target RNA sequence may form a duplex that contains no more than 1, 2, 3, or 4 mismatched or bulging nucleotides over a continuous stretch of at least 10 nt, e.g., between 10-30 nt. In some embodiments a guide strand and a target RNA sequence may form a duplex that contains no more than 1, 2, 3, 4, 5, or 6 mismatched or bulging nucleotides over a continuous stretch of at least 12 nt, e.g., between 10-30 nt. In some embodiments, a guide strand and a target RNA sequence may form a duplex that contains no more than 1, 2, 3, 4, 5, 6, 7, or 8 mismatched or bulging nts over a continuous stretch of at least 15 nt, e.g., between 10-30 nt. In some embodiments, a guide strand and a target RNA sequence may form a duplex that contains no mismatched or bulging nucleotides over a continuous stretch of at least 10 nt, e.g., between 10-30 nt. In some embodiments, between 10-30 nt is 10, 11, 12, 13, 14, 15, 16, 17, 18, 19, 20, 21, 22, 23, 24, 25, 26, 27, 28, 29, or 30 nt.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments, strands may differ by 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., about 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5y with respect to the loop or 3y with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., between 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116 (2): 281-97 (2004); Bartel D P. Cell. 136 (2): 215-33 (2009); Winter, J., et al., Nature Cell Biology 11:228-234 (2009). Artificial miRNA may be designed to take advantage of the endogenous RNAi pathway in order to silence a target RNA of interest. The sequence of such artificial miRNA may be selected so that one or more bulges is present when the artificial miRNA is hybridized to its target sequence, mimicking the structure of naturally occurring miRNA: mRNA hybrids. Those of ordinary skill in the art are aware of how to design artificial miRNA.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also be considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

Antisense oligonucleotides (ASO) are small sequences of DNA or RNA (e.g., about 8-50 base pairs in length) able to target RNA transcripts by Watson-Crick base pairing, resulting in reduced or modified protein expression. In some embodiments, oligonucleotides are unmodified. In other embodiments oligonucleotides include one or more modifications, e.g., to improve solubility, binding, potency, and/or stability of the antisense oligonucleotide. Modified oligonucleotides may comprise at least one modification relative to unmodified RNA or DNA. In some embodiments, oligonucleotides are modified to include internucleoside linkage modifications, sugar modifications, and/or nucleobase modifications. Examples of such modifications are known to those of skill in the art.

In some embodiments the oligonucleotide is modified by the substitution of at least one nucleotide with a modified nucleotide, such that in vivo stability is enhanced as compared to a corresponding unmodified oligonucleotide. In some aspects, the modified nucleotide is a sugar-modified nucleotide. In another aspect, the modified nucleotide is a nucleobase-modified nucleotide.

In some embodiments, oligonucleotides, may contain at least one modified nucleotide analogue. The nucleotide analogues may be located at positions where the target-specific activity, e.g., the splice site selection modulating activity is not substantially affected, e.g., in a region at the 5′-end and/or the 3′-end of the oligonucleotide molecule. In some aspects, the ends may be stabilized by incorporating modified nucleotide analogues.

In some aspects preferred nucleotide analogues include sugar- and/or backbone-modified ribonucleotides (i.e., include modifications to the phosphate-sugar backbone). For example, the phosphodiester linkages of a ribonucleotide may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH— group is replaced by a group selected from H, OR, R, halo, SH, SR, NH2, NHR, NR2 or ON, wherein R is C1-C6 alkyl, alkenyl or alkynyl and halo is F, Cl, Br or I.

In some embodiments, modified oligonucleotides comprise one or more modified nucleosides comprising a modified sugar moiety.

Modified oligonucleotides may comprise one or more nucleosides comprising an unmodified nucleobase. In some embodiments modified oligonucleotides comprise one or more nucleosides comprising a modified nucleobase. In some embodiments, modified oligonucleotides comprise one or more nucleosides that does not comprise a nucleobase.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone.

Also preferred are nucleobase-modified ribonucleotides, i.e., ribonucleotides, containing at least one non-naturally occurring nucleobase instead of a naturally occurring nucleobase. Examples of modified nucleobases include, but are not limited to, uridine and/or cytidine modifications at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine. Oligonucleotide reagents of the invention also may be modified with chemical moieties that improve the in vivo pharmacological properties of the oligonucleotide reagents.

In some embodiments, nucleosides of modified oligonucleotides are linked together using any internucleoside linkage.

Additional modifications are known by those of skill in the art and examples can be found in WO 2019/241648, U.S. Pat. Nos. 10,307,434, 9,045,518, and 10,266,822, each of which is incorporated herein by reference.

Oligonucleotides may be of any size and/or chemical composition sufficient to target a testis specific gene located in a male-specific region of the Y chromosome (MSY) having an X-linked homolog, or the X-linked homolog thereof. In some embodiments, an oligonucleotide is between about 5-300 nucleotides or modified nucleotides. In some aspects an oligonucleotide is between about 10-100, 15-85, 20-70, 25-55, or 30-40 nucleotides or modified nucleotides. In certain aspects an oligonucleotide is between about 15-35, 15-20, 20-25, 25-30, or 30-35 nucleotides or modified nucleotides.

In some embodiments, an oligonucleotide and the target RNA sequence have 100% sequence complementarity. In some aspects, an oligonucleotide may comprise sequence variations, e.g., insertions, deletions, and single point mutations, relative to the target sequence. In some embodiments, an oligonucleotide has at least 70% sequence identity or complementarity to the target RNA. In certain embodiments, an oligonucleotide has at least 70%, 75%, 80%, 85%, 90%, 95%, 97%, 99%, or 100% sequence identity to the target sequence.

The method of administering the agent to the male mammal is not limited and may be any suitable method. In some embodiments, the agent is locally administered to the mammal's reproductive cells.

Agents disclosed herein and/or identified or validated using a method described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular agent and the dosage required for therapeutic efficacy (e.g., the desired degree of sex bias). The methods, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. The term “parenteral” includes intravenous, intramuscular, intraperitoneal, subcutaneous, intraosseus, and intrasternal administration, e.g., by injection or infusion techniques. In some embodiments, a route of administration is parenteral or oral. Optionally, a route or location of administration is selected based at least in part on the location of the male's reproductive cells (e.g., testes). For example, an agent may be administered locally to a target tissue or organ, e.g., a tissue or organ comprising reproductive cells. “Local administration” encompasses (1) administration directly into or near a target tissue or organ, (2) into or near a blood vessel that directly supplies a target tissue or organ, or (3) into a fluid-filled extravascular compartment in fluid communication with the target tissue or organ. “Near” in this context refers to locations up to 1 cm, 5 cm, or 10 cm from an edge or border of the target tissue, organ, or blood vessel. In some embodiments an agent is locally administered to the testes. Other appropriate routes and devices for administering agents will be apparent to one of ordinary skill in the art.

Suitable preparations, e.g., substantially pure preparations, of an active agent may be combined with one or more pharmaceutically acceptable carriers or excipients, etc., to produce an appropriate pharmaceutical composition. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington 's Pharmaceutical Sciences”, E. W. Martin, 19th Ed., 1995, Mack Publishing Co.: Easton, PA, and more recent editions or versions thereof, such as Remington: The Science and Practice of Pharmacy. 21st Edition. Philadelphia, PA. Lippincott Williams & Wilkins, 2005, for additional discussion of pharmaceutically acceptable substances and methods of preparing pharmaceutical compositions of various types).

Some aspects of the present disclosure are directed to compositions (e.g., pharmaceutical compositions including compositions for veterinary use) comprising an agent as described herein.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate, fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic. Pharmaceutical compositions and agents for use in such compositions may be manufactured under conditions that meet standards or criteria prescribed by a regulatory agency such as the USFDA (or similar agency in another jurisdiction) having authority over the manufacturing, sale, and/or use of therapeutic agents. For example, such compositions and agents may be manufactured according to Good Manufacturing Practices (GMP) and/or subjected to quality control procedures appropriate for pharmaceutical agents to be administered to humans.

For oral administration, agents can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For topical applications, pharmaceutical compositions may be formulated in a suitable ointment, lotion, gel, or cream containing the active components suspended or dissolved in one or more pharmaceutically acceptable carriers suitable for use in such composition.

For local administration to the eye, pharmaceutical compositions may be formulated as solutions or micronized suspensions in isotonic, pH adjusted sterile saline, e.g., for use in eye drops, or in an ointment. In some embodiments intraocular administration is used. Routes of intraocular administration include, e.g., intravitreal injection, retrobulbar injection, peribulbar injection, subretinal, sub-Tenon injection, and subconjunctival injection

Pharmaceutical compositions may be formulated for transmucosal or transdermal delivery. For transmucosal or transdermal administration, penetrants appropriate to the barrier to be permeated may be used in the formulation. Such penetrants are generally known in the art. Pharmaceutical compositions may be formulated as suppositories (e.g., with conventional suppository bases such as cocoa butter and other glycerides) or as retention enemas for rectal delivery.

In some embodiments, a pharmaceutical composition includes one or more agents intended to protect the active agent(s) against rapid elimination from the body, such as a controlled release formulation, implant, microencapsulated delivery system, etc. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle-based delivery systems are known in the art for delivery of siRNA. Use of such compositions is contemplated. In some embodiments lipidoid particles are used. In some embodiments non-lipid particles are used. Liposomes or other lipid-based particles can also be used as pharmaceutically acceptable carriers. In some embodiments a macroscopic implant is used to deliver an agent systemically or locally.

In some embodiments, a pharmaceutically acceptable derivative of an agent described herein or identified or validated as described herein, is provided. As used herein, a pharmaceutically acceptable derivative of a particular agent includes, but is not limited to, pharmaceutically acceptable salts, esters, salts of such esters, or any other adduct or derivative which upon administration to a subject in need thereof is capable of providing the compound, directly or indirectly. Thus, pharmaceutically acceptable derivatives can include salts, prodrugs, and/or active metabolites. The term “pharmaceutically acceptable salt” refers to those salts which are, within the scope of sound medical judgment, suitable for use in contact with the tissues of humans and/or lower animals without undue toxicity, irritation, allergic response and the like, and which are commensurate with a reasonable benefit/risk ratio. A wide variety of appropriate pharmaceutically acceptable salts are well known in the art. Pharmaceutically acceptable salts include, but are not limited to, those derived from suitable inorganic and organic acids and bases.

Pharmaceutical compositions are, in at least some embodiments, administered for a time and in an amount sufficient to promote sex biasing of offspring. Efficacy and toxicity of active agents can be assessed by standard pharmaceutical procedures in cell cultures or experimental animals. An effective dose (e.g., sufficient to promote sex biasing of offspring) of an active agent in a pharmaceutical composition may be within a range of about 0.001 to about 100 mg/kg body weight, about 0.01 to about 25 mg/kg body weight, about 0.1 to about 20 mg/kg body weight, about 1 to about 10 mg/kg. Other doses include, for example, about 1 μg/kg to about 500 mg/kg, and about 100 μg/kg to about 5 mg/kg. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the age, body weight, general health of the subject, etc.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier.

It will be understood that a therapeutic regimen may include administration of multiple unit dosage forms over a period of time. In some embodiments, a subject is treated for between 1-7 days. In some embodiments a subject is treated for between 7-14 days. In some embodiments a subject is treated for between 14-28 days. In other embodiments, a longer course of therapy is administered, e.g., over between about 4 and about 10 weeks, 10-26 weeks, 26-52 weeks, or longer. In some embodiments, treatment is continued for 1-5 years, 1-10 years, 1-20 years, or more. A subject may receive one or more doses a day, or may receive doses every other day or less frequently, within a treatment period. Treatment courses may be intermittent. For example, a subject may be treated during sexual maturation.

In some embodiments of the screening methods disclosed herein, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg R P. Design and implementation of high-throughput screening assays. Methods Mol Biol., 565:1-32, 2009 and/or An WF & Tolliday N J., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hüser.

The term “hit” generally refers to an agent that achieves an effect of interest in a screen or assay, e.g., an agent that has at least a predetermined level of modulating effect on cell survival, cell proliferation, gene expression, protein activity, or other parameter of interest being measured in the screen or assay. Test agents that are identified as hits in a screen may be selected for further testing, development, or modification. In some embodiments a test agent is retested using the same assay or different assays. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a non-target molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties. An analog that has one or more improved properties may be identified and used in a composition or method described herein. In some embodiments a molecular target of a hit compound is identified or known. In some embodiments, additional compounds that act on the same molecular target may be identified empirically (e.g., through screening a compound library) or designed.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert (ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate agent is identified, additional agents, e.g., analogs, may be generated based on it. An additional agent, may, for example, have increased cell uptake, increased potency, increased stability, greater solubility, or any improved property. In some embodiments a labeled form of the agent is generated. The labeled agent may be used, e.g., to directly measure binding of an agent to a molecular target in a cell. In some embodiments, a molecular target of an agent identified as described herein may be identified. An agent may be used as an affinity reagent to isolate a molecular target. An assay to identify the molecular target, e.g., using methods such as mass spectrometry, may be performed. Once a molecular target is identified, one or more additional screens maybe performed to identify agents that act specifically on that target.

Any of a wide variety of agents may be used as a test agent in various embodiments. For example, a test agent may be a small molecule, polypeptide, peptide, amino acid, nucleic acid, oligonucleotide, lipid, carbohydrate, or hybrid molecule. In some embodiments a nucleic acid used as a test agent comprises a siRNA, shRNA, antisense oligonucleotide, aptamer, or random oligonucleotide. In some embodiments a test agent is cell permeable or provided in a form or with an appropriate carrier or vector to allow it to enter cells.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells. In some embodiments, if the agent is one that is found in a cell culture medium known or used in the art, the agent may be used at a different, e.g., higher, concentration when used as a test agent in a method or composition described herein.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

Studies of Y-chromosome evolution have focused primarily on gene decay, a consequence of suppression of crossing over with the X chromosome. Here is provided evidence that suppression of X-Y crossing over unleashed a second dynamic: selfish X-Y arms races that reshaped the sex chromosomes in mammals as different as cattle, mice, and men. Using super-resolution sequencing, the Y chromosome of Bos taurus (bull) is found to be dominated by lineage-specific massive amplification of testis-expressed gene families, making it the most gene-dense Y chromosome sequenced to date. As in mice, an X-linked homolog of a bull Y-amplified gene has become testis-specific and amplified. This striking evolutionary convergence suggests that lineage-specific X-Y co-evolution through gene amplification, and the selfish forces underlying this phenomenon, are dominatingly powerful among diverse mammalian lineages. Together with Y gene decay, X-Y arms races molded mammalian sex chromosomes and influenced the course of mammalian evolution.

The inventors assembled the complete sequence of the Bos taurus (i.e. domestic cattle) Y chromosome and part of the X chromosome. The inventors discovered an X-Y gene pair, expressed exclusively in the testis, that is coamplified on the X and Y chromosomes. There are 79 copies of HSFY on the Y chromosome and at least 11 copies of HSFX on the X chromosome. This is only the second case of massive amplification of a testis-specific gene family on mammalian X and Y chromosomes. During sequencing of the mouse Y (published in 2014), it was discovered that an unrelated gene family—Slx/Sly—is testis-specific and has undergone co-amplification on the X and Y chromosomes. The inventors proposed that this X-Y co-evolution may be a manifestation of sex-linked meiotic drive, where the X and Y chromosomes compete for transmission to gametes during the process of meiosis. In mouse, it has been shown that altering the copy number of Sly and Slx changes the sex ratio, such that reduced Sly copy number results in more female offspring and reduced Slx copy number results in more male offspring. The inventors propose that the same situation might be true in cattle, and manipulation of the copy number of HSFY and HSFX could result in sex ratio skewing.

### Introduction

Mammalian sex chromosomes evolved from an ordinary pair of autosomes (1-3). Differentiation of the X and Y chromosomes began 200-300 million years ago, propelled by gradual suppression of X-Y crossing over, most likely due to inversions on the Y chromosome (3, 4). While the X chromosome continues to participate in crossing over with its homolog during female meiosis, the male-specific region of the Y chromosome (MSY) lacks a partner for crossing over during male meiosis. Theoretical and empirical studies of Y chromosome evolution have largely focused on one specific outcome of crossover suppression-genetic decay. Gene loss is a nearly universal feature of MSY evolution, and has been reported in species as diverse as plants (papaya, white campion, and heartwing sorrel), flies, fish, frogs, lizards, and mammals (6-14).

Complete sequencing of four mammalian Y chromosomes—human, chimpanzee (Pan troglodytes), rhesus macaque (Macaca mulatta), and mouse (Mus musculus)—revealed that MSY decay has been counterbalanced to varying degrees by sequence acquisition and amplification (15-18). The super-resolution sequencing method employed for these MSY sequences is a requirement for elucidating the complex structure of ampliconic sequences, which are euchromatic repeats that display >99% identity over more than 10 kb. The mouse MSY stands in stark contrast to the three primate MSYs because it has been overtaken by ampliconic sequence. Nearly 98% of the mouse MSY is ampliconic, compared to 45%, 57%, and 5% in human, chimpanzee, and rhesus, respectively. The amplified mouse sequence bears no homology to any primate MSY sequence and contains three gene families that are expressed exclusively in the male germline. These MSY amplified genes have independently acquired and amplified germline-specific gene families on the mouse X chromosome. Mouse X-Y co-amplification may be a manifestation of sex-linked meiotic drive, where the X and Y chromosomes are competing against each other for transmission to the next generation (18).

Sex-linked meiotic drive has been reported in dozens of insect species but, in mammals, this phenomenon has only been observed in a few rodent species (19). In natural mouse populations, autosomes are known to engage in meiotic drive, with the t-haplotypes of chromosome 17 harboring a strong segregation distorter (20). It is unclear what features insects and rodents share that make them uniquely permissible to the proliferation of meiotic drive systems.

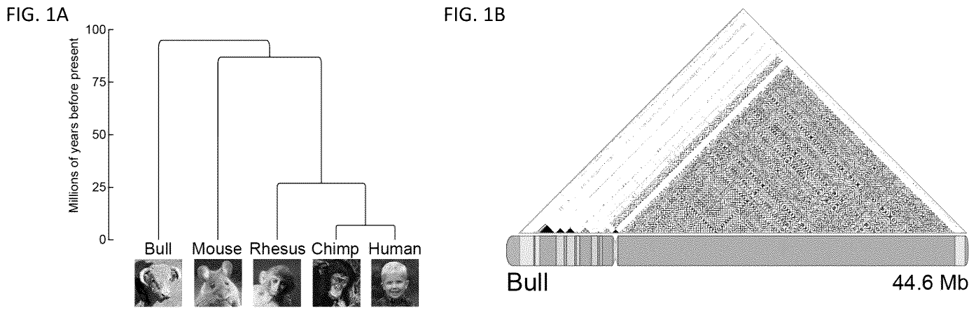

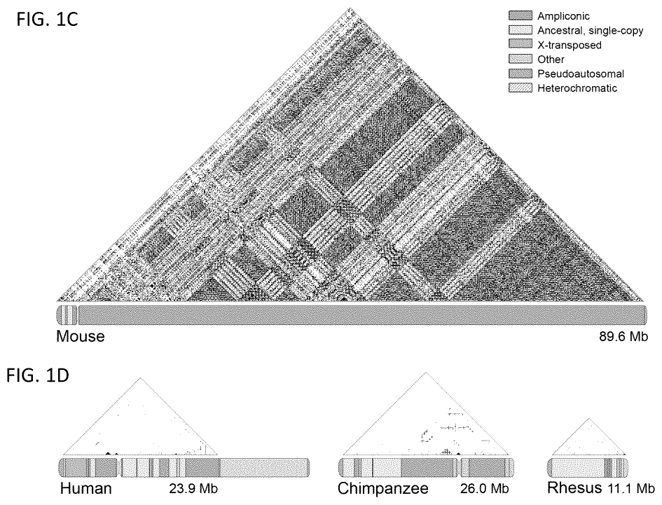

Here is presented the complete MSY sequence of the bull (Bos taurus, Hereford breed). This species represents a third major branch of the mammalian tree (FIG. 1A) and is of great agricultural, anthropological, and economic importance. The bull and mouse lineages diverged from each other nearly 100 million years ago, and bull and mouse differ profoundly in life histories and reproductive strategies. However, their MSYs exhibit striking structural convergence, which has important implications for our understanding of mammalian MSY evolution and biology. As in mouse, the majority of the bull MSY is comprised of massively amplified sequence containing testis-specific genes that are expressed in the male germline. Consequently, the bull and mouse MSYs are much larger in size and have substantially higher gene densities than sequenced primate MSYs. Homologous ampliconic sequence were found in numerous species of wild bovids, dating the origin of this sequence to at least 17 million years ago. Finally, as in mouse, evidence for independent amplification of related testis-specific gene families on the X and Y chromosomes of bull were found. These findings are evidence that sex-linked meiotic drive is more common and widespread among mammals than previously thought. Thus, suppression of X-Y crossing over not only leads to genetic decay, but it may foster selfish X-Y arms races, a phenomenon that may have widespread consequences throughout the genome across mammals.

### Results

Mapping, sequencing, and assembly of the bull MSY

The inventors produced and assembled a nearly complete sequence of the bull MSY (FIG. 1B), accurate to one nucleotide per megabase (Mb). To achieve this high level of completeness and accuracy, the super-resolution Single-Haplotype Iterative Mapping and Sequencing (SHIMS) methodology was used (21), which was previously employed to sequence sex chromosomes in human, chimpanzee, rhesus macaque, mouse, and chicken (2, 15-18, 22, 23). A total of 40 Mb of sequence was assembled from a tiling path of 349 bull Bacterial Artificial Chromosome (BAC) clones, all derived from a single male donor (L1 Domino, Bos taurus, Hereford breed). The assembly contains 15 gaps, 12 of which are associated with heterochromatin or tandem arrays. Fluorescence in situ hybridization (FISH) was used to visualize these tandem arrays, to order and orient sequence contigs within the assembly, and to estimate gap sizes within single-copy regions). Where possible, linkage between contigs was also confirmed using radiation hybrid mapping. The approximate location of the centromere in our assembly was determined using a combination of FISH, immunocytochemistry, and radiation hybrid mapping. Estimated gap sizes were incorporated into a model bull MSY sequence that spans 44 Mb; all of the subsequent analyses are based on this sequence assembly.

### Comparison of Five Sequenced MSYs Reveals Convergent Evolution in Bull and Mouse

The analysis of the mouse MSY revealed that it followed an evolutionary path distinct from that of the three sequenced primate MSYs: human, chimpanzee and rhesus macaque. The primate MSYs are by far the smallest chromosomes in their respective genomes; their euchromatic sequence content ranges from 12 Mb in rhesus to 26 Mb in chimpanzee. By contrast, the euchromatic sequence content of the mouse MSY totals nearly 90 Mb (18). The size disparity between the mouse and primate MSYs is due solely to massive amplification of lineage-specific sequences in mouse, since ancestral X-homologous regions of the MSY, presumed to be present in the mammalian ancestor, are markedly reduced in mouse. With only four complete MSY sequences in hand, it was impossible to know if this rampant MSY sequence expansion is a peculiarity of the mouse lineage.

The bull MSY sequence suggests that massive MSY sequence amplification may be a broad characteristic of mammalian MSYs. The bull MSY is more similar in structure to the mouse MSY than to any of the primate MSYs (FIG. 1B-D). Single-copy X-homologous regions in bull and mouse account for only 4% and 2% of their respective MSY euchromatin contents, compared to 37%, 33%, and 86% in human, chimpanzee, and rhesus, respectively. The long arm of the bull MSY, which spans roughly 35 Mb, is comprised almost entirely of ampliconic sequence, with four basic repeat units. The mouse MSY long-arm ampliconic sequence is 86 Mb in length and contains three basic repeat units. There is no homology between the ampliconic sequences in bull and mouse, indicating that these amplifications were independent. The bull amplicons contain two distinct multi-copy gene families: HSFY, which is found in primates and originates from the ancestral autosome pair that gave rise to the mammalian X and Y (24), and ZNF280BY, which is lineage-specific and originates from an autosome-to-Y transposition event (25). The bull MSY amplicons also contain about 400 inactivated pseudogenes. By comparison, the mouse MSY amplicons contain three lineage-specific gene families that were acquired by the Y chromosome during rodent evolution. The inventors conclude that the MSYs in the bull and mouse lineages experienced extensive but independent amplifications.

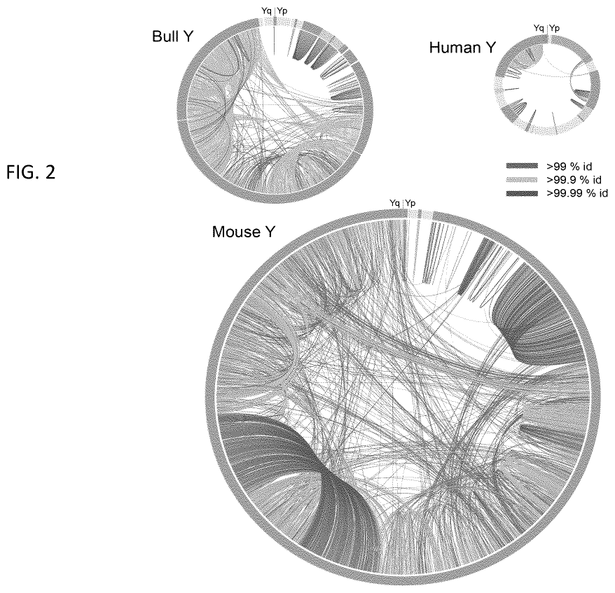

In all of the sequenced MSYs, ampliconic regions display extremely high inter-repeat nucleotide identities-up to 99.99%-because of ongoing gene conversion (as well as other forms of homologous recombination) between repeat units (26). Both the bull and mouse MSYs have ample substrates for gene conversion, so maps of intrachromosomal identities across these chromosomes were generated to visualize the extent of such sequence homogenization in bull and mouse (FIG. 2). Overall, the long-arm amplicons in bull are more homogeneous than in mouse, as evidenced by a higher density of >99.9% matches in the intrachromosomal identity map (FIG. 2). Compared to human (as a representative primate), both bull and mouse MSY regions exhibit high sequence identity spanning great distances-often tens of megabases-implying that gene conversion is not constrained by physical distance between substrates (FIG. 2).

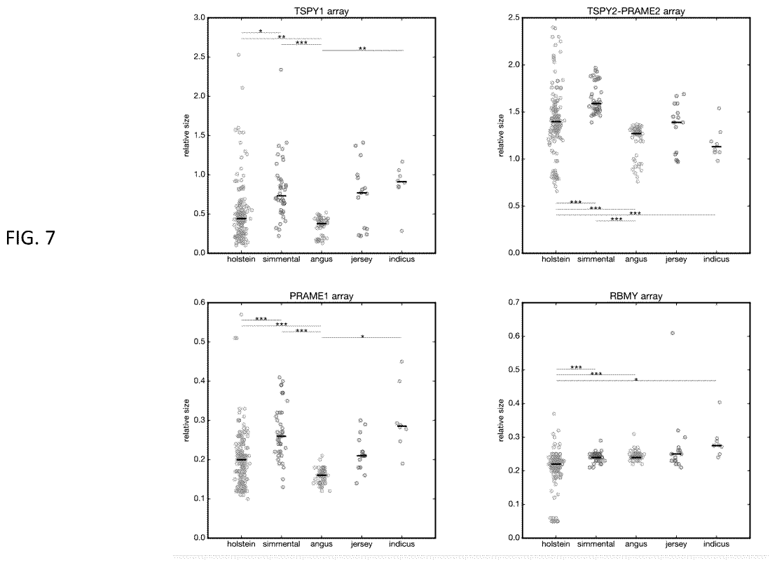

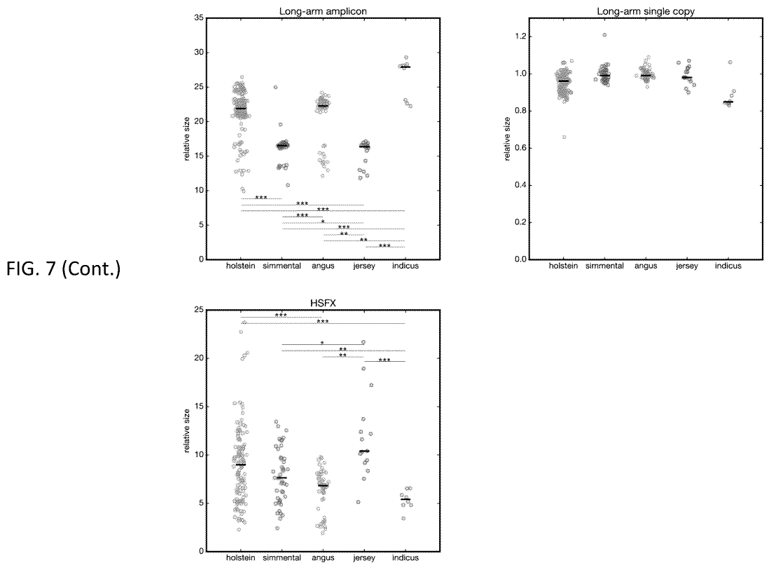

While gene conversion, which is homologous recombination without crossing-over, between MSY amplicons results in sequence homogenization, homologous recombination with crossing-over can create inversions, duplications, and deletions, with duplications and deletions in ampliconic regions giving rise to copy number variation (27). To examine copy number variation within bull MSY amplicons, >200 publicly available bull whole-genome shotgun sequences were analyzed from four different breeds (28). Using read-depth mapping of raw sequencing data, it was determined that copy-number variability is indeed prevalent among bull MSY amplicons (FIG. 7). Among Holstein cattle, for example, the copy number of the TSPY2-PRAME2 array varies nearly five-fold, and the copy number of the long-arm ampliconic sequence varies nearly three-fold.

### Evolutionary Conservation of Bull MSY Ampliconic Regions

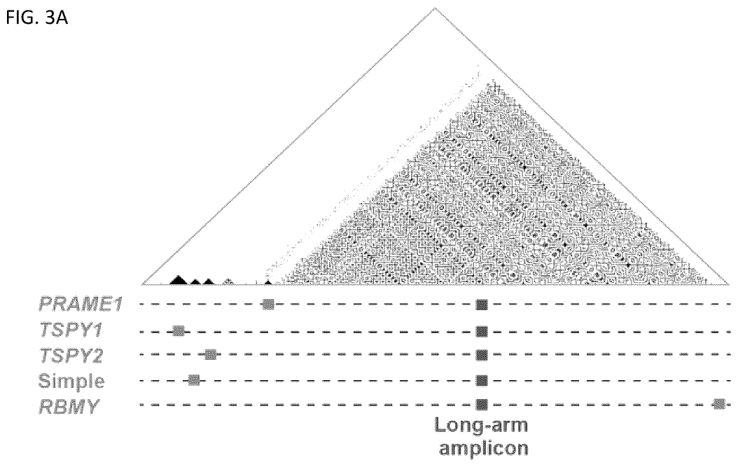

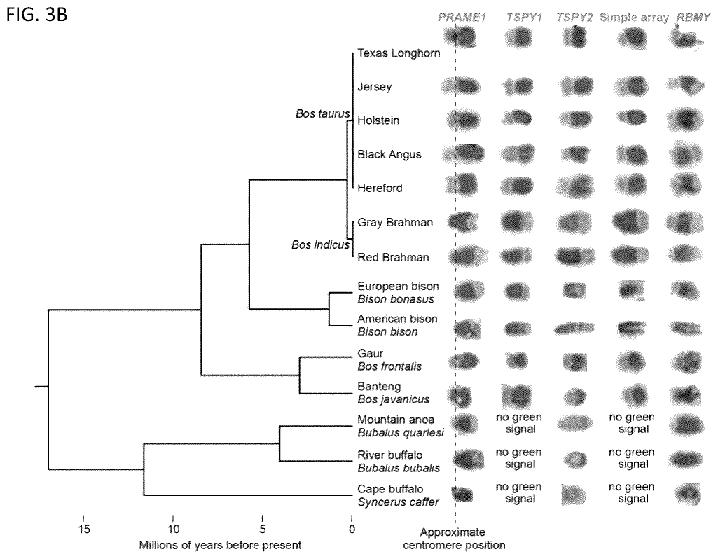

The MSYs of bull and mouse differ radically in structure from the sequenced primate MSYs; one common feature that distinguishes these two disparate species from the three primates is domestication. It was previously demonstrated that the MSY of a Mus spretus-derived strain, diverged from Mus musculus three million years ago, carries ampliconic sequences homologous to the long-arm amplicons found in the sequenced MSY (from Mus musculus musculus, strain C57BL/6), ruling out the possibility that amplification was a consequence of selective breeding (18). Similarly, the inventors sought to determine if the bull MSY amplification predated the domestication of cattle. FISH analysis was used to examine six different ampliconic regions on the bull MSY, including the long-arm amplicon, across nine bovine species spanning 17 million years of evolution (29-31). The long-arm amplicon, as well as three other tandem arrays, are conserved and amplified on the MSY in all nine species (FIG. 3). Although the arrangement and size of the amplicons differs between distantly related species, the presence of these sequences in diverse species of wild bovids confirms that these amplification events are not a consequence of selective breeding by humans. Large-scale repeats, such as those that comprise the bull MSY sequence, are prone to rearrangement, so the conservation of these evolutionarily volatile sequences across millions of years of evolution implies the action of purifying selection.

### Gene Content of Bull MSY

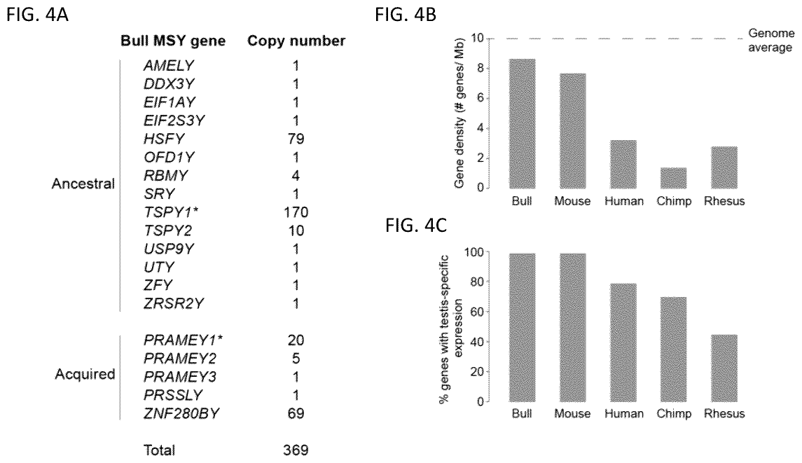

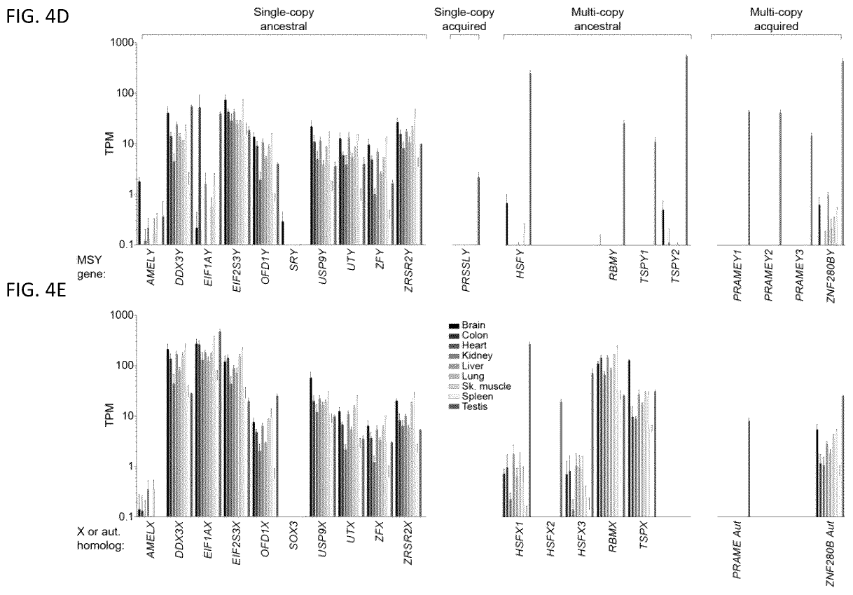

A second feature that distinguishes the bull and mouse MSYs from those of primates is their high gene densities, a consequence of the massive amplification of genes within their ampliconic regions (FIG. 4) (18). In bull, the MSY gene density approaches the genome average of 10 per Mb (32), counter to previous generalizations that Y chromosomes are poor in genes (33). Bull and mouse have fewer ancestral genes—13 and 9 unique genes, respectively—than human and rhesus—with 17 and 18 genes, respectively (11). Chimpanzee contains only 13 intact ancestral genes because of lineage-specific gene losses (34, 35). The bull pseudoautosomal region (PAR) is much larger than the PARs of primates (36), and three genes that are male-specific in human remain pseudoautosomal in cattle, accounting for some of the disparity in MSY ancestral gene content.

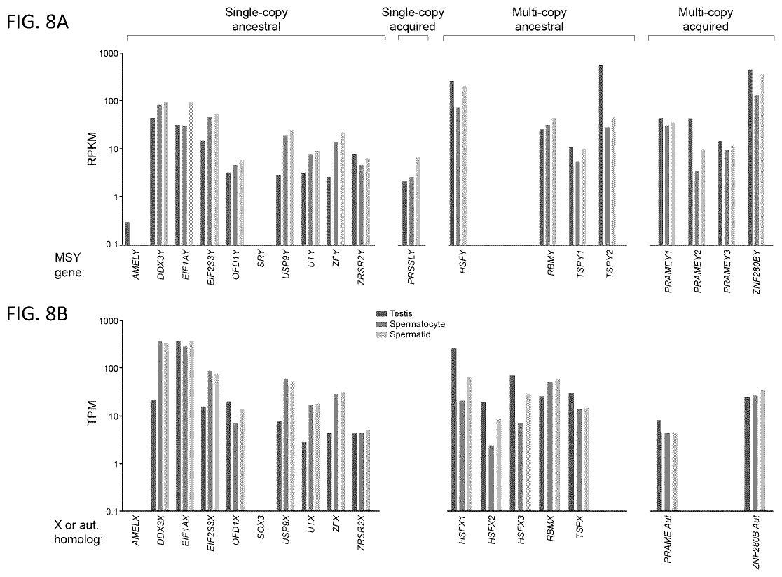

In all previously sequenced MSYs (human, chimpanzee, rhesus, and mouse), it was found that multicopy genes were expressed exclusively or predominantly in testes, while most single-copy ancestral genes were found to be expressed more broadly (15-17, 37). Y-linked testis-specific genes likely have specialized functions related to spermatogenesis, while the broadly expressed genes have more widespread functions related to gene regulation (11). The inventors analyzed the expression of bull MSY genes, as well as their homologs on the X-chromosome or autosomes, across eight somatic tissues and testis using previously published datasets (38). All multicopy bull MSY genes, both ancestral and acquired, displayed largely testis-specific expression patterns (FIG. 4D) (11). Because of these patterns of gene expression, the bull and mouse MSYs as a whole can be viewed as more skewed towards testis-specific expression (FIG. 4C). Using a germ-cell depleted mouse model, Applicants were able to refine the expression pattern of the mouse MSY multicopy gene families further, finding that most are expressed exclusively in germ cells (18). Although a similar model is not available for bull, Applicants analyzed previously published RNA-seq datasets generated from purified germ cells (pachytene spermatocytes and round spermatids) (39), and were able to detect transcription of bull MSY genes and their X-chromosome and autosome counterparts in these samples (FIG. 8), providing evidence that these genes are transcribed in male germ cells. By contrast, most single-copy ancestral genes of the bull MSY are expressed at appreciable levels in all tissues examined, as are their X-linked homologs (FIG. 4D,E).

### X-Y Co-Amplification in Bull

The final common feature shared between the bull and mouse MSYs—the co-amplification of X and Y gene families—has the greatest biological implications. The three acquired, amplified gene families on the mouse MSY long arm have non-allelic homologs on the X chromosome that have been amplified in parallel, and all of these sex-linked gene families are expressed exclusively in the male germline (18). It was speculated that this X-Y co-evolution may be a manifestation of sex-linked meiotic drive, which distorts the balanced ratio of male and female offspring.

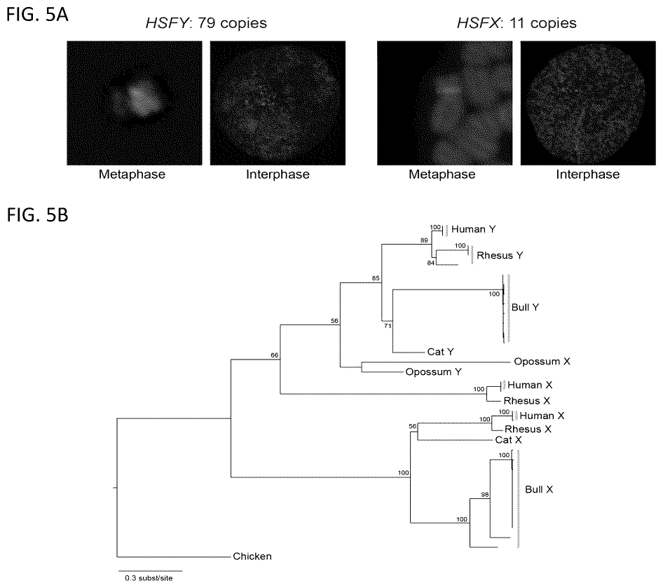

X-Y co-amplification is also found in cattle. One of the amplified gene families on the bull MSY long arm is HSFY, which is present in at least 79 copies. HSFY is also multicopy in human and rhesus, but with only two or three copies, respectively. The X homolog of this gene, HSFX, is highly amplified in bull (FIG. 5A). Although the Bos taurus reference X-chromosome assembly contains just one copy of HSFX (32), this analysis revealed higher than average BAC coverage within this region. SHIMS was used to sequence a total of eight X-chromosome clones, and found that HSFX is in fact a multicopy gene in Bos taurus. There are three distinct HSFX variants in the bull, and one variant has undergone further amplification, with at least nine highly similar copies (FIG. 5B). Interestingly, HSFX is also multicopy in human and rhesus: two variants are highly diverged from each other, and in human, each variant is duplicated (FIG. 5B). In bull, HSFY and HSFX copies display high levels of intra-family nucleotide identity, implying ongoing intrachromosomal gene conversion, but the Y- and X-linked families are highly diverged from each other-their predicted proteins exhibit only 34% amino acid identity-indicating that there is no ongoing X-Y recombination at these loci (FIG. 5B). The amplification of HSFY and HSFX therefore appears to have occurred independently on the bovine Y and X chromosomes, and phylogenetic analysis shows that the same is true in primates (FIG. 5B). HSFY and HSFX are expressed predominantly or exclusively in the testis in both human and bull, and are expressed in male germ cells in bull (FIG. 4, FIG. 8), mirroring the expression pattern of the sex-linked amplified gene families in mouse. The bull provides a second example of rampant amplification on the MSY accompanied by large-scale X-linked amplification of homologous gene families, demonstrating that these events are linked and may be widespread among mammalian lineages.

### Discussion

The bull MSY is the seventh sex chromosome to be sequenced using the SHIMS approach, the only proven strategy for producing super-resolution assemblies of ampliconic regions. Of the finished sex chromosomes, the bull MSY is second only to the mouse MSY in total ampliconic sequence content, and SHIMS was vital for the elucidation of the complex repeat architecture of the bull Y long arm and an accurate representation of its gene content. Because the majority of ampliconic repeats comprising the bull Y long arm differ by less than one base pair (bp) per 1000 (FIG. 2), a whole genome shotgun approach, where read lengths are typically <500 bp in length, would collapse the majority of amplicons into a single unit. Thus, the ampliconic repeats of the bull MSY, which are of considerable biological interest, were only accessible through SHIMS.

Until now, the mouse MSY stood alone, contrasting starkly with the three sequenced primate MSYs in terms of overall size and structure, leaving open the question of whether the comparatively large and mostly ampliconic mouse MSY was unique among mammals. The MSY of bull, which represents a third major branch of the mammalian tree, is decidedly more similar to the MSY of mouse, demonstrating that massive lineage-specific MSY amplification of testis-specific gene families is not a peculiarity of the mouse lineage, but is widely distributed among mammals. More limited sequencing surveys of additional mammalian MSYs—including pig, gorilla, cat, and horse—show evidence for large-scale amplification of testis-specific gene families as well (40-43). Horse, which has multiple Y- and X-linked copies of a parasite-derived, testis-specific transcript, may provide another example of X-Y coamplification (42). To understand the extent, mechanism, and evolutionary trajectory of MSY amplification, complete SHIMS-based assemblies of ampliconic regions from these and other mammals will be required.

The MSY amplification of testis-specific gene families in both bull and mouse occurred in parallel with amplifications of related testis-specific gene families on the X chromosome (FIG. 5) (18). It was previously hypothesized that X-vs.-Y meiotic drive may induce an evolutionary arms race between the sex chromosomes in mouse (18). In this scenario, the proportion of female and male offspring is influenced by a meiotic “driver” and “suppressor” located on the X and Y chromosomes, respectively (44, 45). This proposal is based on prior observations of such phenomena in other animals: sex-chromosome-associated meiotic drive has been documented at least 24 times in insects and at least three times in rodents (19). These two groups of species share certain features, such as large brood size, large effective population size, and high population density, whereas cattle are typically uniparous with a large body size and relatively small effective population size (46).

In addition to HSFX/Y, there is another example of X-Y co-amplification in humans (Table 1).

TABLE 1
Extent of co-amplification of testis-specific gene families
on X and Y chromosomes in bull, mouse, and human.

| | % of X or Y | | | | | |
|---|---|---|---|---|---|---|
| | euchromatin occupied | | X-Y co- | | Total copy | |
| | by X-Y co- | | amplified | | number of X-Y co- | |
| | amplified sequence | | gene families | | amplified genes | |
| | X | Y | X | Y | X | Y |
| Bull | 2 | 82 | HSFX | HSFY | 11 | 79 |
| Mouse | 8 | 96 | Sstx, | Ssty, | 64 | 629 |
| | | | Slx, | Sly, | | |
| | | | Srsx | Srsy | | |
| Human | 0.06 | 1.65 | HSFX | HSFY | 4 | 2 |
| | 0.04 | 0.33 | VCX | VCY | 4 | 2 |

There are multiple copies of VCY (2 copies) and VCX (4 copies) in both human and chimpanzee, but no other primates. Both gene families are expressed exclusively in the male germline: VCX encodes a highly acidic protein while VCY encodes a highly basic protein, suggestive of antagonistic functions (47). The extent of X-Y co-amplification varies widely in these three examples. Roughly 96% of the mouse MSY euchromatic sequence arose as a consequence of this phenomenon, compared to less than 2% of the human MSY (Table 1). In bull, mouse, and human, the X chromosome sequence has not been affected to as large a degree as the Y chromosome (Table 1), perhaps because the requirement for recombination between X homologs during female meiosis serves to constrain runaway amplification. One study found intense positive selection and selective sweeps associated with numerous X-chromosome ampliconic regions in primates (48). Y-linked antagonists of these drivers are not necessarily homologous loci, so this phenomenon may have shaped the evolution of both X and Y chromosomes more deeply and extensively than is immediately apparent.

In mouse, this “arms-race” hypothesis is supported by knockdown studies where disruption of the X-linked homologs of one co-amplified gene family—Slx/Slx1—causes sex-ratio skewing towards males, while disruption of the Y-linked homologs—Sly—causes sex ratio skewing towards females (49, 50). Moreover, targeted deletion and duplication of the Slx/Slx1 gene family skews sex ratios towards males and females, respectively (51). While no comparable functional studies exist for cattle, examination of >200 bull genomes revealed that the long-arm amplicon varies considerably in size within and between breeds (FIG. 7). Further investigations may reveal a correlation between long-arm amplicon copy number and offspring sex ratio in cattle. Naturally occurring or artificially induced perturbations of sex ratios in cattle would be of great interest, both biologically and commercially.

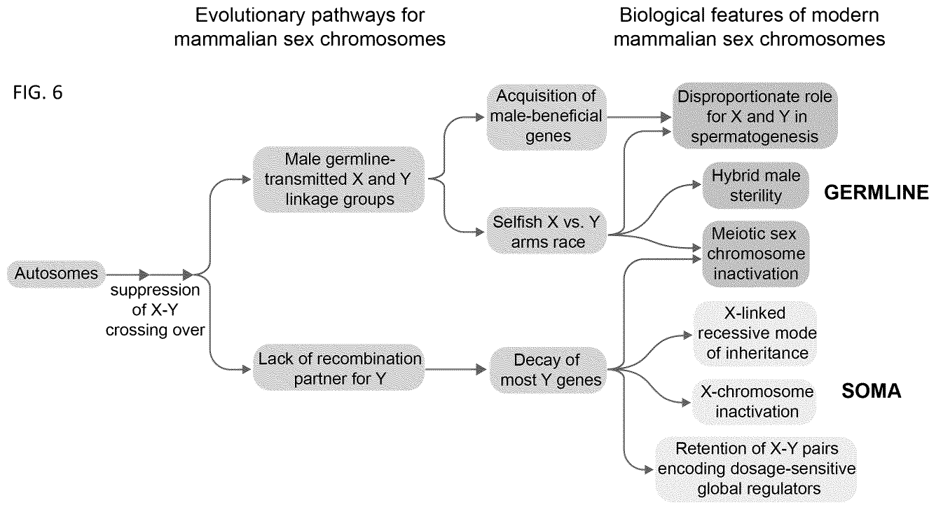

The bull Y chromosome sequence provides a second example of massive amplification driven by X-Y co-evolution in mammals, substantiating a second major theme of mammalian Y chromosome evolution, and one that is distinct from the process of Y decay (FIG. 6). Both of these evolutionary processes—decay and amplification—stem from the same initial processes in sex chromosome evolution: large-scale inversions on the Y chromosome suppressing crossing over with the X chromosome. The cascading effects of this X-Y divergence reach far beyond the sex chromosomes and have been influenced differently in the soma and germline. Massive loss of Y chromosome genes forms the basis for the X-linked recessive model of inheritance, where X-linked recessive alleles lack “sheltering” from wild-type homologs in males. A second consequence of Y decay that played out in the soma is X-chromosome inactivation, which is the manifestation of a complex evolutionary process involving Y gene loss, consequent upregulation of the X homolog in males and females, and silencing of one X-linked copy in females (1, 52). By contrast, the germline fostered selfish evolutionary processes. Suppression of X-Y crossing over essentially creates distinct X and Y linkage groups in the male germline, producing an attractive environment for factors involved in meiotic drive (19, 53). The downstream consequences of sex-linked meiotic drive include X-Y incompatibilities that manifest as hybrid male sterility, promoting speciation (54, 55), and the evolution of meiotic sex chromosome inactivation, which may constrain X-vs.-Y competition during spermatogenesis through transcriptional silencing of the sex chromosomes (45, 56).

### Methods

### BAC Selection and Sequencing

The Single-Haplotype Iterative Mapping and Sequencing (SHIMS) strategy (57) was used to assemble a path of sequenced clones selected from the CHORI-240 BAC library (bapac.chori.org) and a custom BAC library (BTDAEX) constructed by Amplicon Express (Pullman, WA). Both libraries are derived from a single male donor (L1 Domino, Bos taurus Hereford breed). Fingerprint contigs and end sequences had been generated previously for the CHORI-240 library (58), so the inventors relied primarily upon this library. The BTDAEX library was used to fill gaps. BAC tiling paths were selected for sequencing as previously described (15). The error rate in the finished sequence was estimated by counting mismatches in alignments between overlapping clones.

### Assessing Copy Number Variability

Illumina sequence data generated from 234 bull genomes from four different Bos taurus breeds (28) were obtained from NCBI (project accession number SRP039339). Male Bos taurus indicus whole genome Illumina sequence data was also obtained from NCBI to use as an outgroup (project accession numbers PRJNA277147, PRJNA324270, and PRJNA324822). Bowtie was used to map reads (59), allowing up to two mismatches and up to a 650-bp insert size for paired-end reads; data from each animal was analyzed separately. First, reads were mapped to our entire bull MSY assembly, and only mapped reads were analyzed further. Next, these reads were mapped to the female Bos taurus genome (bosTau4), and mapped reads removed. The remaining reads were then mapped to five different MSY ampliconic regions (TPSY1 array, TSPY2-PRAME2 array, PRAME1 array, RBMY array, and long-arm amplicon) and to 1-Mb single-copy regions from both the MSY short and long arms. These reads were also mapped to HSFX and to a single-copy region on the X chromosome as a control. Bowtie parameters for this step were adjusted to allow up to 500 alignments per read and to prioritize the best alignments. The size of each ampliconic region was calculated relative to the 1-Mb single-copy short-arm region based on comparative read depths. The statistical significance of differences between means was determined using Welch's t-test.

### Fluorescence In Situ Hybridization (FISH)

FISH on male cell lines derived from various cattle breeds was performed as previously described (60). A minimum of 20 images for metaphase and 50 images for interphase were obtained for each experiment.

### Radiation Hybrid Mapping

25 STS markers were tested on a 25,000-rad panel consisting of 132 hybrid clones. A cell line derived from the same donor animal (L1 Domino) as the BAC library was used to construct the panel. Data analyses were performed using RHMAPPER 1.22 (61).

### Dotplot and Phylogenetic Analyses

Triangular and square dotplot analyses were performed using custom Perl codes (available at pagelab.wi.mit.edu/software). Circos plots were generated as previously described (62). For each MSY sequence, step-wise 50-kb segments were used as BLAST queries against the remainder of the unmasked MSY sequence, and non-gapped percent identities were calculated for top-hit alignments. For bull and human, tandem array-associated gaps (three in bull and two in human) are represented by model sequence. For phylogenetic analyses, alignments were generated using PRANK (63), and trees were generated using PhyML with default parameters (64). HSFY/X sequences used in this analysis can be found below.

### Gene Expression Analyses

To generate the histograms shown in FIG. 4D,E, a total of 27 publicly available datasets were reanalyzed (38). These comprised three datasets each from the following Bos taurus tissues: brain, colon, heart, kidney, liver, lung, skeletal muscle, spleen, and testes (accession numbers SRX196344-SRX196370). After the initial mapping, it was noted that one of the skeletal muscle samples (accession number SRX196368) indicated strong expression of several ampliconic genes—HSFY, TSPY, and ZNF280BY—which are usually testis-specific. It is suspected that this dataset might be mislabeled or contaminated, so 11 additional male Bos taurus skeletal muscle RNA-seq datasets were analyzed (accession numbers SRX317172-SRX317179, SRX317191-SRX317193). no transcription of HSFY, TSPY, and ZNF280BY were found in any of the 11 other muscle datasets. It was therefore concluded that the skeletal muscle dataset corresponding to accession number SRX196368 was mislabeled or contaminated and removed it from the gene expression analyses. For analysis of bull MSY genes in relation to ampliconic repeat architecture, RNA-seq was performed on samples derived from Bos taurus (Hereford breed) whole testis and isolated pachytene spermatocytes and round spermatids. Purified germ cell fractions were obtained as previously described (65). Total RNA was isolated from samples using the RNeasy kit (Qiagen, Hilden, Germany) and sequencing libraries were made using the Tru-Seq RNA kit (Illumina, San Diego, CA). Using the Illumina HiSeq 2500 platform, 100-bp paired-end reads were obtained for testis and 40-bp paired-end reads were obtained for purified germ cells. For all datasets, RNA-seq reads were mapped to the Bos taurus transcriptome (Bos_taurus.ARS-UCD1.2; MSY genes manually added) using Kallisto with sequence-based bias correction (66). For multi-copy X and Y gene families, the number of reads that mapped to any single member of the gene family were summed. Plots with means and standard errors were generated using Prism 8.

### Sequence Annotation

Interspersed repeats were electronically identified using RepeatMasker (www.repeatmasker.org). Protein-coding genes were identified as previously described (17). Active genes were distinguished from pseudogenes by 1) evidence for transcriptional activity by RT-PCR or RNA-seq, 2) intact splice sites (if multi-exonic), and 3) full-length open reading frames (compared to other members of Y-linked multi-copy gene family, Y-homologs in other species, or X/autosomal homologs in Bos taurus). Loci with confirmed transcription but without significant ORFs were considered non-coding transcripts.

TABLE 2
GenBank accession numbers and references for bull MSY genes

| Gene | Accession | |
|---|---|---|
| name | number | Reference |
| AMELY | NM_174240.2 | Gibson C., et al. Biochemistry 30: 1075 (1991). |
| DDX3Y | FJ659845.1 | Gotherstrom A., et al. Proc Royal Society B |
| | | 272: 2345 (2005) |
| EIF1AY | NM_001145757.1 | Van Laere, A. S., et al. Genome Res 18: 1884 |
| | | (2008) |
| EIF2S3Y | FJ627276.1 | This disclosure |
| HSFY | NM_001077006.1 | Moore et al., Direct GenBank submission: |
| | | BC103406 (2007) |
| OFDIY | JN193532.1 | Van Laere, A.S., et al. Genome Res 18: 1884 |
| | | (2008) |
| PRAMEY1 | NM_001245953.1 | Chang, T. C., et al. PLoS ONE 6: E16867 (2011) |
| PRAMEY2 | GU144302.1 | Chang, T. C., et al. PLoS ONE 6: E16867 (2011) |
| PRAMEY3 | XM_003587135.2 | This disclosure |
| RBMY | GU304599.2 | Bellott, D. W., et al. Nature 508: 494 (2014) |
| SRY | NM_001014385.1 | Payen, E. J. and Cotinot, C. Y. Nucleic Acids Res |
| | | 21: 2772 (1993) |
| PRSSLY | KC120771.1 | This disclosure |
| TSPY1 | JN585955.1 | Jakubiczka, S., et al. Genomics 3: 732 (1993) |
| TSPY2 | JN585956.1 | This disclosure |
| USP9Y | FJ627275.1 | Van Laere, A. S., et al. Genome Res 18: 1884 |
| | | (2008) |
| UTY | FJ627278.1 | This disclosure |
| ZFY | NM_177491.1 | Aasen, E. and Medrano, J. F. Biotechnology |
| | | 8: 1279 (1990) |
| ZNF280BY | NM_001078120.1 | Yang, Y., et al. BMC Genomics 12: 13 (2011) |
| ZRSR2Y | GAPO01000004.1 | Hamilton, C. K., et al. Theriogenology 77: 1587 |
| | | (2012) |

Accession numbers for confirmed cDNA sequences:
Human HSFY1 NM_033108
Human HSFY2 NM_153716
Human HSFX1 NM_016153
Human HSFX2 NM_001164415
Human HSFX3 NM_001323079
Human HSFX4 NM_001351114
Rhesus HSFY FJ527015
Bull HSFY NM_001077006
Cat HSFY NM_001040123
Opossum HSFY GQ253469
Opossum HSFX GQ253474

Accession numbers for predicted genes:
Rhesus HSFX1 XM_005594782
Rhesus HSFX3 XM_001089561
Chicken HSFXL XM_416447

Predicted cat HSFX cDNA sequence obtained from whole-genome assembly (felCat5):

>cat X
(SEQ ID NO: 1)
TGAGACATTCTCATGGCAAAAGTGCTCTTGGTCTAGACTAGC
TCCATGGCTAGTCAGAGTGCTAAGGAGATACCCAAAGGGAAGCTGGCCCC
ATCTGATGATGGAGAGCCAGCACCAGAATTACCATCTAGTTCATCCCAGG
ATCCAAATTTGGATTCCGGGGAGATTTTGGTGATGAATAGGGACCAAGCA
GTGATCCAAGATCCAGGCCCCCAAGACAACCCACAACCACAGGCCCCAAA
CCAAGGCGCCGCCAACGTGGGAGAAAACAACAGTATTCTTGGGCTCTCCT
TCCCAAGAAAGCTTTGGATGATCCTGGAGGACAACACCTTCACGTCCGTG
CGCTGGAACGATGCCGGGGACACCGTGATCATCGACGAAGACCTTTTCCA
GAGGGAGGTTCTTCACCGCAGAGGCGCGGAGAGAATCTTTGAAACTGACA
GCTTGAAGACTTTCATCCGCCTGCTGAACCTGTACGGCTTCAGCAAAATAC
GCGATTTACCGCAACGCCAACTTTCAGAGAGACAAGCCTTTCCTGCTCAG
GAACATTCAGAGGAAAAGTGACCTGAGAGTCACCACCACCTGGCTAGGCA
CCAGTGCACCAACTCCAAAGAGAAAGAAGCCGGTGGCAGCAACAAGACA
GTCCCCACGAATCCATCACGAGGAACCCGCCAACGACGACAAGACGGTCC
TCGGCGCAGCCCCAAATGCTCAGGGTCCCAGCAGCAGCCAGCCCTTCGCC
TTCTCTGGCATTTGGTCTCTGAGCAGCGCAGCCGGGTATGCCATGGCAACT
CATGGCCCGAGTGAGCCAGGTGGCCTGAGTGGGGAGGGCACCTCCAGGA
ACATGATGTTTGTGCCCCTGGCTACTGCCAGAAGGGATGACACAGGGGAA
CTGCCCGTCAGCCCCCCAGTTTACCCCGACTATGGTACAGTGATGTCTCTG
TATAACACCTGTTATTCCATCCTGCTGGCAGCCCTGTCAGTCATGTCCCCA
AATGAGGCCCCCAGTGAGAACGAGGAGCAGGAGGGCTCCTCAGATTACA
AGTGTGCCCTCTGTGAACATTTCAAGGAAAATCCAGGTCCCTAGGCTCAC
AGGCCACTGATGACAGCTAAAAAGTCACCACTGTAACCATCAATCTATTT
TCGGCTTTAATGGAAACAAACACAACTCCGCGGGAGTAAATAAACAGTC

Predicted bull HSFX cDNA sequences obtained from sequencing non-overlapping CHORI-240 BACs:

>Hsfx.1 84P17
(SEQ ID NO: 2)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCAGTGTACCCAGATTACGAATCGGTGATGGCTTTGTACAACACCTG
TTACTCCATCCTGATGGCGGGCCTTTTAGTCATGGCACCAGATGAGGCCCC
TGAGGCGGAGGATGAGCAGGGAGAGTCCTCACATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGA
>Hsfx.2 120M19
(SEQ ID NO: 3)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGATAGACAG
CATCAAGAGCTTCATCCGTGAACTGAATCTGTACGGGTTCAGGAAAGTCC
GCCCTTCGAGTCACTCTACAGGGAAGAAGCTCATGATCTATCGAAACTCC
AATTTTCAGAGAGACAAGCCTCTCCTTCTGCATAACATCCAGAGGAAAGG
CAACCCCAGAACAACTTCTCAGCCTGCCACTGGCACAACAACTCCAAAGA
GAAAGAAGCGAGTGGTGGCAACCAGACACTCTCCTCAATTCCACCACAAC
GAGTTCACCCAAAAGGCTGGCAACAAAGTCCAGAAGGGGATGCCAACTG
CTCGCAGAACCCCCAGCCAGTGCTCATTTGTGTTCTCTGACCTGTCTGTGG
GCAGTGTAGCCAGGCGGGCTGGGGAAAACCATCTCCCCAGTGAGCAGGG
CAGCCCCAGCAGGGCGGCTGGAGAGGGCACATCCAGCAATGCCATATCTG
TGCCCTCGGCTACTGCTGAAAGGGACAGCCCAGGGAAACTGCCCGAGAGC
CCCCCGGTGTACCCAGATTACGAATCGGTGATGGCTTTGTACAACACCTGT
TACTCCATCCTGATGGCGGGCCTTTTAGTCATGGCACCAGATGAGGCCCCT
GAGGCGGAGGATGAGCAGGGAGAGTCCTCACATTATAAGTGTGCCCTCTG
TGAGCAGCTCAAGAACAAGCCCAATCCCTGAGCTGCCAGATACCTAGGGA
CACTCAAAAGCACTCTCTTATAATTAAAAATAGATTTCTGGCTCTAATAAA
GTCAAGCAAAACTCCATTGGAGTAAATAAACAATCAAACGA
>Hsfx.3 79D24
(SEQ ID NO: 4)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
TGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATAGC
TGTGCCCTCGGCTACTGCTGAAAGGGACAGCCCAGGGAAACTGCCCGAGA
GCCCCCCGGTGTACCCAGATTACGAATCGGTGATGGCTTTGTACAACACCT
GTTACTCCATCCTGATGGCGGGCCTTTTAGTCATGGCACCAGATGAGGCCC
CTGAGGCGGAGGATGAGCAGGGAGAGTCCTCACATTATAAGTGTGCCCTC
TGTGAGCAGCTCAAGAACAAGCCCAATCCCTGAGCTGCCAGATACCTAGG
GACACTCAAAAGCACTCTCTTATAATTAAAAATAGATTTCTGGCTCTAATA
AAGTCAAGCAAAACTCCATTGGAGTAAATAAACAATCAAACGA
>Hsfx.4 31M19
(SEQ ID NO: 5)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCGGTGTACCCAGATTACGAATCGGTGATGGCTTTGTACAACACCTG
TTACTCCATCCTGATGGCGGGCCTTTTAGTCATGGCACCAGATGAGGCCCC
TGAGGCGGAGGATGAGCAGGGAGAGTCCTCACATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGA
>Hsfx.5 107K24
(SEQ ID NO: 6)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCGGTGTACCCAGATTACGAATCGGTGATGGCTTTGTACAACACCTG
TTACTCCATCCTGATGGCGGGCCTTTTAGTCATGGCACCAGATGAGGCCCC
TGAGGCGGAGGATGAGCAGGGAGAGTCCTCACATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGAG
>Hsfx.6 392J12
(SEQ ID NO: 7)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGGGAGGCTTTGGAGAAGCAGGGTGACCAGCCCA
AGAGCCCAGATCCAGGCCTCTATGACAATCCACTCCAACAGGGCCCAAAC
CCAGAAATGGCCAAAGAGGAAGAGAATAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAACCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCGGTGTACCCAGATTACGAATCGGTGATGGCTTTGTACAACACCTG
TTACTCCATCCTGATGGCGGGCCTTTTAGTCATGGCACCAGATGAGACCCC
TGAGGCGGAGGATGAGCAGGGAGAGTCCTCAGATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGA
>Hsfx.7 54K1
(SEQ ID NO: 8)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCGGTGTACCCAGATTACGAATCGGTGATGGCTTTGTACAACACCTG
TTACTCCATCCTGATGGCGGGCCTTTTAGTCATGGCACCAGATGAGGCCCC
TGAGGCGGAGGATGAGCAGGGAGAGTCCTCACATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGA
>Hsfx.8 176L20
(SEQ ID NO: 9)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCGGTGTACCCAGATTACGAATCGGTGATGGCTTTGTACAACACCTG
TTACTCCATCCTGATGGCGGGCCTTTTAGTCATGGCACCAGATGAGGCCCC
TGAGGCGGAGGATGAGCAGGGAGAGTCCTCACATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGA
>Hsfx.9 437020
(SEQ ID NO: 10)
GAGTATTTATGGGGCACCCCGACCATGGTCTGAGGCCCGTTT
TCCCTGAGCACTCACTGCTTGGATAGATAATGCTCCTTAGCTAGACTGGCT
CCATGGCTAGTCAGAGTTCCCATGAGGCACGGGCAGCCCTGCTGATCCTA
TCAACTGATGGGGAGCCTGCAGCAGGGGACACCCGTGATTCCTCCCCAGA
TCCAAACCTGGATTTAGGAGAGGCTTTGGAGAAGCAGGGTGACCAGCCCA
AGAGCCCAGATCCAGGCCTCCATGACAATCCACTCCAACAGGGCCCAAAC
CCAGAAATGGCCAAAGAGGAAGAGAACAACGCCATCCTTGGGCTGTCCTT
CCCCAGGAAGCTCTGGAGGATTGTGGAGGATGCAGCCTTCACCTCTGTGC
ACTGGAACGATGAGGGAGATACAGTGGTCATCGAGGCAGATCTCTTCCAG
ATAGAGGTACTCCAGCGCAGAGGCATGGACCAGATCTTCGAGACAGACA
GCATCAAGAGCTTCATCCGTGAACTGAATCTGTACGGGTTCAGGAAAGTC
CGCCCTTCGAGTCACTCTACAGGGAAGAAGCTCATGATCTATCGAAACTC
CAATTTTCAGAGAGACAAGCCTCTCCTTCTGCATAACATCCAGAGGAAAG
GCAACCCCAGAACAACTTCTCAGCCTGCCACTGGCACAACAACTCCAAAG
AGAAAGAAGCGAGTGGTGGCAACCAGACACTCTCCTCAATTCCACCACAA
CGAGTTCACCCAAAAGGCTGGCAACAAAGTCCAGAAGGGGATGCCAACT
GCTCGCAGAACCCCCAGCCAGTGCTCATTTGTGTTCTCTGACCTGTCTGTG
GGCAGTGTAGCCAGGCGGGCTGGGGAAAACCATCTCCCCAGTGAGCAGG
GCAGCCCCAGCAGGGCGGCTGGAGAGGGCACATCCAGCAATGCCATATCT
GTGCCCTCGGCTACTGCTGAAAGGGACAGCCCAGGGAAACTGCCCGAGAG
CCCCCCGGTGTACCCAGATTACGAATCGGTGATGGCTTTGTACAACACCTG
TTACTCCATCCTGATGGCGGGCCTTTTAGTCATGGCACCAGATGAGGCCCC
TGAGGCGGAGGATGAGCAGGGAGAGTCCTCACATTATAAGTGTGCCCTCT
GTGAGCAGCTCAAGAACAAGCCCAATCCCTGAGCTGCCAGATACCTAGGG
ACACTCAAAAGCACTCTCTTATAATTAAAAATAGATTTCTGGCTCTAATAA
AGTCAAGCAAAACTCCATTGGAGTAAATAAACAATCAAACGA

### REFERENCES

1. Ohno S. Sex Chromosomes and Sex-linked Genes. Berlin: Springer-Verlag; 1967.
2. Bellot D W, Skaletsky H, Pyntikova T, Mardis E R, Graves T, Kremitzki C, Brown L G, Rozen S, Warren W C, Wilson R K, Page D C. Convergent evolution of chicken Z and human X chromosomes by expansion and gene acquisition. Nature (2010) 466:612-6.
3. Lahn B T, Page D C. Four evolutionary strata on the human X chromosome. Science (1999) 286:964-7.
4. Ross M T, Grafham D V, Coffey A J, Scherer S, McLay K, Muzny D, Platzer M, Howell G R, Burrows C, Bird C P, Frankish A, Lovell F L, Howe K L, Ashurst J L, Fulton R S, Sudbrak R, Wen G, Jones M C, Hurles M E, Andrews T D, Scott C E, Searle S, Ramser J, Whittaker A, Deadman R, Carter N P, Hunt S E, Chen R, Cree A, Gunaratne P, Havlak P, Hodgson A, Metzker M L, Richards S, Scott G, Steffen D, Sodergren E, Wheeler D A, Worley K C, Ainscough R, et al. The DNA sequence of the human X chromosome. Nature (2005) 434:325-37.
5. Bachtrog D. Y-chromosome evolution: emerging insights into processes of Y-chromosome degeneration. Nat Rev Genet (2013) 14:113-24.
6. Chibalina M V, Filatov D A. Plant Y chromosome degeneration is retarded by haploid purifying selection. Curr Biol (2011) 21:1475-9.
7. Hough J, Hollister J D, Wang W, Barrett S C, Wright S I. Genetic degeneration of old and young Y chromosomes in the flowering plant Rumex hastatulus. PNAS (2014) 111:7713-8.
8. Kaiser V B, Zhou Q, Bachtrog D. Nonrandom gene loss from the Drosophila miranda neo-Y chromosome. Genome Biol Evol (2011) 3:1329-37.
9. Miura I, Ohtani H, Ogata M. Independent degeneration of W and Y sex chromosomes in frog Rana rugosa. Chromosome Res (2012) 20:47-55.
10. Zhou Q, Zhu H M, Huang Q F, Zhao L, Zhang G J, Roy S W, Vicoso B, Xuan Z L, Ruan J, Zhang Y, Zhao R P, Ye C, Zhang X Q, Wang J, Wang W, Bachtrog D. Deciphering neo-sex and B chromosome evolution by the draft genome of Drosophila albomicans. BMC Genomics (2012) 13:109.
11. Bellott D W, Hughes J F, Skaletsky H, Brown L G, Pyntikova T, Cho T J, Koutseva N, Zaghlul S, Graves T, Rock S, Kremitzki C, Fulton R S, Dugan S, Ding Y, Morton D, Khan Z, Lewis L, Buhay C, Wang Q, Watt J, Holder M, Lee S, Nazareth L, Alfoldi J, Rozen S, Muzny D M, Warren W C, Gibbs R A, Wilson R K, Page D C. Mammalian Y chromosomes retain widely expressed dosage-sensitive regulators. Nature (2014) 508:494-9.
12. Liu Z, Moore P H, Ma H, Ackerman C M, Ragiba M, Yu Q, Pearl H M, Kim M S, Charlton J W, Stiles J I, Zee F T, Paterson A H, Ming R. A primitive Y chromosome in papaya marks incipient sex chromosome evolution. Nature (2004) 427:348-52.
13. Lisachov A P, Makunin A I, Giovannotti M, Pereira J C, Druzhkova A S, Caputo Barucchi V, Ferguson-Smith M A, Trifonov V A. Genetic content of the neo-sex chromosomes in Ctenonotus and Norops (Squamata, Dactyloidae) and degeneration of the Y chromosome as revealed by high-throughput Sequencing of individual chromosomes. Cytogenet Genome Res (2019) 157:115-22.
14. Peichel C L, McCann S R, Ross J A, Naftaly A F S, Urton J R, Cech J N, Grimwood J, Schmutz J, Myers R M, Kingsley D M, White M A. Assembly of the threespine stickleback Y chromosome reveals convergent signatures of sex chromosome evolution. Genome Biol (2020) 21:177.
15. Hughes J F, Skaletsky H, Brown L G, Pyntikova T, Graves T, Fulton R S, Dugan S, Ding Y, Buhay C J, Kremitzki C, Wang Q, Shen H, Holder M, Villasana D, Nazareth L V, Cree A, Courtney L, Veizer J, Kotkiewicz H, Cho T J, Koutseva N, Rozen S, Muzny D M, Warren W C, Gibbs R A, Wilson R K, Page D C. Strict evolutionary conservation followed rapid gene loss on human and rhesus Y chromosomes. Nature (2012) 483:82-6.
16. Hughes J F, Skaletsky H, Pyntikova T, Graves T A, van Daalen S K, Minx P J, Fulton R S, McGrath S D, Locke D P, Friedman C, Trask B J, Mardis E R, Warren W C, Repping S, Rozen S, Wilson R K, Page D C. Chimpanzee and human Y chromosomes are remarkably divergent in structure and gene content. Nature (2010) 463:536-9.
17. Skaletsky H, Kuroda-Kawaguchi T, Minx P J, Cordum H S, Hillier L, Brown L G, Repping S, Pyntikova T, Ali J, Bieri T, Chinwalla A, Delehaunty A, Delehaunty K, Du H, Fewell G, Fulton L, Fulton R, Graves T, Hou S F, Latrielle P, Leonard S, Mardis E, Maupin R, McPherson J, Miner T, Nash W, Nguyen C, Ozersky P, Pepin K, Rock S, Rohlfing T, Scott K, Schultz B, Strong C, Tin-Wollam A, Yang S P, Waterston R H, Wilson R K, Rozen S, Page D C. The male-specific region of the human Y chromosome is a mosaic of discrete sequence classes. Nature (2003) 423:825-37.
18. Soh Y Q, Alfoldi J, Pyntikova T, Brown L G, Graves T, Minx P J, Fulton R S, Kremitzki C, Koutseva N, Mueller J L, Rozen S, Hughes J F, Owens E, Womack J E, Murphy W J, Cao Q, de Jong P, Warren W C, Wilson R K, Skaletsky H, Page D C. Sequencing the mouse Y chromosome reveals convergent gene acquisition and amplification on both sex chromosomes. Cell (2014) 159:800-13.
19. Jaenike J. Sex chromosome meiotic drive. Annu Rev Ecol Syst (2001) 32:25-49.
20. Silver L M. Mouse t haplotypes. Annu Rev Genet (1985) 19:179-208.
21. Bellott D W, Cho T J, Hughes J F, Skaletsky H, Page D C. Cost-effective high-throughput single-haplotype iterative mapping and sequencing for complex genomic structures. Nat Protoc (2018) 13:787-809.
22. Mueller J L, Skaletsky H, Brown L G, Zaghlul S, Rock S, Graves T, Auger K, Warren W C, Wilson R K, Page D C. Independent specialization of the human and mouse X chromosomes for the male germ line. Nat Genet (2013) 45:1083-7.
23. Bellott D W, Skaletsky H, Cho T J, Brown L, Locke D, Chen N, Galkina S, Pyntikova T, Koutseva N, Graves T, Kremitzki C, Warren W C, Clark A G, Gaginskaya E, Wilson R K, Page D C. Avian W and mammalian Y chromosomes convergently retained dosage-sensitive regulators. Nat Genet (2017) 49:387-94.
24. Hamilton C K, Revay T, Domander R, Favetta L A, King W A. A large expansion of the HSFY gene family in cattle shows dispersion across Yq and testis-specific expression. PLOS One (2011) 6: e17790.
25. Yang Y, Chang T C, Yasue H, Bharti A K, Retzel E F, Liu W S. ZNF280B Y and ZNF280A Y: autosome derived Y-chromosome gene families in Bovidae. BMC Genomics (2011) 12:13.
26. Rozen S, Skaletsky H, Marszalek J D, Minx P J, Cordum H S, Waterston R H, Wilson R K, Page D C. Abundant gene conversion between arms of palindromes in human and ape Y chromosomes. Nature (2003) 423:873-6.
27. Lange J, Noordam M J, van Daalen S K, Skaletsky H, Clark B A, Macville M V, Page D C, Repping S. Intrachromosomal homologous recombination between inverted amplicons on opposing Y-chromosome arms. Genomics (2013) 102:257-64.
28. Daetwyler H D, Capitan A, Pausch H, Stothard P, van Binsbergen R, Brondum R F, Liao X, Djari A, Rodriguez S C, Grohs C, Esquerre D, Bouchez O, Rossignol M N, Klopp C, Rocha D, Fritz S, Eggen A, Bowman P J, Coote D, Chamberlain A J, Anderson C, VanTassell C P, Hulsegge I, Goddard M E, Guldbrandtsen B, Lund M S, Veerkamp R F, Boichard D A, Fries R, Hayes B J. Whole-genome sequencing of 234 bulls facilitates mapping of monogenic and complex traits in cattle. Nat Genet (2014) 46:858-65.
29. Bradley D G, MacHugh D E, Cunningham P, Loftus R T. Mitochondrial diversity and the origins of African and European cattle. PNAS (1996) 93:5131-5.
30. Hernandez Fernandez M, Vrba E S. A complete estimate of the phylogenetic relationships in Ruminantia: a dated species-level supertree of the extant ruminants. Biol Rev Camb Philos Soc (2005) 80:269-302.
31. Loftus R T, MacHugh D E, Bradley D G, Sharp P M, Cunningham P. Evidence for two independent domestications of cattle. PNAS (1994) 91:2757-61.
32. Bovine Genome S, Analysis C, Elsik C G, Tellam R L, Worley K C, Gibbs R A, Muzny D M, Weinstock G M, Adelson D L, Eichler E E, Elnitski L, Guigo R, Hamernik D L, Kappes S M, Lewin H A, Lynn D J, Nicholas F W, Reymond A, Rijnkels M, Skow L C, Zdobnov E M, Schook L, Womack J, Alioto T, Antonarakis S E, Astashyn A, Chapple C E, Chen H C, Chrast J, Camara F, Ermolaeva O, Henrichsen C N, Hlavina W, Kapustin Y, Kiryutin B, Kitts P, Kokocinski F, Landrum M, Maglott D, Pruitt K, et al. The genome sequence of taurine cattle: a window to ruminant biology and evolution. Science (2009) 324:522-8.
33. Mank J E. Small but mighty: the evolutionary dynamics of W and Y sex chromosomes. Chromosome Res (2012) 20:21-33.
34. Perry G H, Tito R Y, Verrelli B C. The evolutionary history of human and chimpanzee Y-chromosome gene loss. Mol Biol Evol (2007) 24:853-9.
35. Hughes J F, Skaletsky H, Pyntikova T, Minx P J, Graves T, Rozen S, Wilson R K, Page D C. Conservation of Y-linked genes during human evolution revealed by comparative sequencing in chimpanzee. Nature (2005) 437:100-3.
36. Das P J, Chowdhary B P, Raudsepp T. Characterization of the bovine pseudoautosomal region and comparison with sheep, goat, and other mammalian pseudoautosomal regions. Cytogenet Genome Res (2009) 126:139-47.
37. Bentley D R, Balasubramanian S, Swerdlow H P, Smith G P, Milton J, Brown C G, Hall K P, Evers D J, Barnes C L, Bignell H R, Boutell J M, Bryant J, Carter R J, Keira Cheetham R, Cox A J, Ellis D J, Flatbush M R, Gormley N A, Humphray S J, Irving L J, Karbelashvili M S, Kirk S M, Li H, Liu X, Maisinger K S, Murray L J, Obradovic B, Ost T, Parkinson M L, Pratt M R, Rasolonjatovo I M, Reed M T, Rigatti R, Rodighiero C, Ross M T, Sabot A, Sankar S V, Scally A, Schroth G P, Smith M E, et al. Accurate whole human genome sequencing using reversible terminator chemistry. Nature (2008) 456:53-9.
38. Merkin J, Russell C, Chen P, Burge C B. Evolutionary dynamics of gene and isoform regulation in mammalian tissues. Science (2012) 338:1593-9.
39. Lesch B J, Silber S J, McCarrey J R, Page D C. Parallel evolution of male germline epigenetic poising and somatic development in animals. Nat Genet (2016) 48:888-94.
40. Skinner B M, Lachani K, Sargent C A, Yang F, Ellis P, Hunt T, Fu B, Louzada S, Churcher C, Tyler-Smith C, Affara N A. Expansion of the HSFY gene family in pig lineages: HSFY expansion in suids. BMC Genomics (2015) 16:442.
41. Brashear W A, Raudsepp T, Murphy W J. Evolutionary conservation of Y Chromosome ampliconic gene families despite extensive structural variation. Genome Res (2018) 28:1841-51.
42. Janecka J E, Davis B W, Ghosh S, Paria N, Das P J, Orlando L, Schubert M, Nielsen M K, Stout T A E, Brashear W, Li G, Johnson C D, Metz R P, Zadjali A M A, Love C C, Varner D D, Bellott D W, Murphy W J, Chowdhary B P, Raudsepp T. Horse Y chromosome assembly displays unique evolutionary features and putative stallion fertility genes. Nat Commun (2018) 9:2945.
43. Tomaszkiewicz M, Rangavittal S, Cechova M, Campos Sanchez R, Fescemyer H W, Harris R, Ye D, O'Brien P C, Chikhi R, Ryder O A, Ferguson-Smith M A, Medvedev P, Makova K D. A time- and cost-effective strategy to sequence mammalian Y Chromosomes: an application to the de novo assembly of gorilla Y. Genome Res (2016) 26:530-40.
44. Presgraves D C. Sex chromosomes and speciation in Drosophila. Trends Genet (2008) 24:336-43.
45. Meiklejohn C D, Tao Y. Genetic conflict and sex chromosome evolution. Trends Ecol Evol (2010) 25:215-23.
46. MacEachern S, Hayes B, McEwan J, Goddard M. An examination of positive selection and changing effective population size in Angus and Holstein cattle populations (Bos taurus) using a high density SNP genotyping platform and the contribution of ancient polymorphism to genomic diversity in Domestic cattle. BMC Genomics (2009) 10:181.
47. Lahn B, Page D. A human sex-chromosomal gene family expressed in male germ cells and encoding variably charged proteins. Hum Mol Genet (2000) 9:311-9.
48. Nam K, Munch K, Hobolth A, Dutheil J Y, Veeramah K R, Woerner A E, Hammer M F, Great Ape Genome Diversity P, Mailund T, Schierup M H. Extreme selective sweeps independently targeted the X chromosomes of the great apes. PNAS (2015) 112:6413-8.
49. Cocquet J, Ellis P J, Mahadevaiah S K, Affara N A, Vaiman D,

Burgoyne P S. A genetic basis for a postmeiotic X versus Y chromosome intragenomic conflict in the mouse. PLOS Genet (2012) 8: e1002900.
50. Cocquet J, Ellis P J, Yamauchi Y, Mahadevaiah S K, Affara N A, Ward M A, Burgoyne P S. The multicopy gene Sly represses the sex chromosomes in the male mouse germline after meiosis. PLOS Biol (2009) 7: e1000244.
51. Kruger A N, Brogley M A, Huizinga J L, Kidd J M, de Rooij D G, Hu Y C, Mueller J L. A neofunctionalized X-linked ampliconic gene family is essential for male fertility and equal sex ratio in mice. Curr Biol (2019) 29:3699-706 e5.
52. Jegalian K, Page D C. A proposed path by which genes common to mammalian X and Y chromosomes evolve to become X inactivated. Nature (1998) 394:776-80.
53. Lyttle T W. Segregation distorters. Ann Rev Genet (1991) 25:511-57.
54. Hurst L D, Pomiankowski A. Causes of sex ratio bias may account for unisexual sterility in hybrids: a new explanation of Haldane's rule and related phenomena. Genetics (1991) 128:841-58.
55. Frank S A. Divergence of meiotic drive-suppression systems as an explanation for sex-biased hybrid sterility and inviability. Evolution (1991) 45:262-7.
56. Ellis P J, Clemente E J, Ball P, Toure A, Ferguson L, Turner J M, Loveland K L, Affara N A, Burgoyne P S. Deletions on mouse Yq lead to upregulation of multiple X- and Y-linked transcripts in spermatids. Human Mol Genet (2005) 14:2705-15.
57. Kuroda-Kawaguchi T, Skaletsky H, Brown L G, Minx P J, Cordum H S, Waterston R H, Wilson R K, Silber S, Oates R, Rozen S, Page D C. The AZFc region of the Y chromosome features massive palindromes and uniform recurrent deletions in infertile men. Nat Genet (2001) 29:279-86.
58. Snelling W M, Chiu R, Schein J E, Hobbs M, Abbey C A, Adelson D L, Aerts J, Bennett G L, Bosdet I E, Boussaha M, Brauning R, Caetano A R, Costa M M, Crawford A M, Dalrymple B P, Eggen A, Everts-van der Wind A, Floriot S, Gautier M, Gill C A, Green R D, Holt R, Jann O, Jones S J, Kappes S M, Keele J W, de Jong P J, Larkin D M, Lewin H A, McEwan J C, Mckay S, Marra M A, Mathewson C A, Matukumalli L K, Moore S S, Murdoch B, Nicholas F W, Osoegawa K, Roy A, Salih H, et al. A physical map of the bovine genome. Genome Biol (2007) 8: R165.
59. Langmead B, Trapnell C, Pop M, Salzberg S L. Ultrafast and memory-efficient alignment of short DNA sequences to the human genome. Genome Biol (2009) 10: R25.
60. Raudsepp T, Chowdhary B P. FISH for mapping single copy genes. Methods Mol Biol (2008) 422:31-49.
61. Slonim D, Kruglyak L, Stein L, Lander E. Building human genome maps with radiation hybrids. J Comput Biol (1997) 4:487-504.
62. Krzywinski M, Schein J, Birol I, Connors J, Gascoyne R, Horsman D, Jones S J, Marra M A. Circos: an information aesthetic for comparative genomics. Genome Res (2009) 19:1639-45.
63. Loytynoja A. Phylogeny-aware alignment with PRANK.

### Methods Mol Biol (2014) 1079:155-70.

64. Guindon S, Delsuc F, Dufayard J F, Gascuel O. Estimating maximum likelihood phylogenies with PhyML. Methods Mol Biol (2009) 537:113-37.
65. Bellve A R. Purification, culture, and fractionation of spermatogenic cells. Methods Enzymol (1993) 225:84-113.
66. Bray N L, Pimentel H, Melsted P, Pachter L. Near-optimal probabilistic RNA-seq quantification. Nat Biotechnol (2016) 34:525-7.

### Example 2

### Abstract

Meiotic drivers are selfish genetic elements that promote the transmission of the chromosome that carries them. When meiotic drive elements reside on sex chromosomes, they skew the sex ratio, resulting in selection for an unlinked suppressor to restore an equal sex ratio. Sex-chromosome meiotic drive elements have been identified in a number of insect, rodent, and plant species, but are usually species-specific, implying their transient nature. Applicants discovered that an ancient, broadly conserved gene-PRSSLY (protease, serine-like Y), located on the Y chromosome in eutherian mammals-appears to be a meiotic drive factor because it is essential for equal sex ratio in mice. Applicants show that Prssly knockouts produce offspring with a female-biased sex ratio. Across eutheria, PRSSLY's expression is testis-specific, and, in mouse, it is most robustly expressed in post-meiotic germ cells. PRSSLY homologs are found on the X chromosome and autosomes in more distant animals, including marsupials, monotremes, lizards, newts, and caecilians. Notably, PRSSLY is absent from some eutherian groups, including primates and felines. The closest paralog to PRSSLY is the autosomal gene PRSS55, which is expressed exclusively in testes, involved in sperm differentiation and migration, and essential for male fertility in mice. If the function of PRSSLY is conserved in diverse eutheria, then it would be the first example of a sex-linked meiotic drive element conserved for >100 million years.

### Introduction

Selfish genetic elements exist for the sole purpose of self-promotion in their host genome. Meiotic drivers are a particular class of selfish elements with the ability to promote the non-Mendelian transmission of the chromosome where they reside (1). Meiotic drive elements can arise on autosomes or sex chromosomes, but appear to be more frequent on the latter. This suggests that sex chromosomes are a favorable target, or that sex-linked drivers are more frequently identified because they result in sex-ratio skewing, which is a readily observable outcome of their appearance (2).

Although apparently more common than its autosomal counterpart, sex-linked meiotic drive can have dire consequences. Sex-ratio distortion results in more frequent production of offspring of the more common sex, which lessens the likelihood of their future mating success, and if strong enough, sex-ratio skewing can eventually lead to extinction (1, 3). Therefore, suppressors of sex-chromosome drivers, which can appear on the opposite sex chromosome or on autosomes, are strongly favored by selection (1-3). These drive-suppression systems may cycle in and out of populations, with the loss of drivers leading to relaxed selection for suppressor maintenance (4). Support for the relatively short-lived nature of sex-linked meiotic drive systems comes from their disparate species distribution. They are largely species-specific and limited to a handful of Drosophila, fly, mosquito, butterfly, rodent, and plant species (2).

Applicants identified a widespread yet uncharacterized mammalian Y-linked gene—PRSSLY (protease, serine-like Y)—that may function in meiotic drive mechanisms, suggesting that sex-linked meiotic drive may be more widespread than previously appreciated. PRSSLY escaped notice until recently because it is not present on the first three Y chromosomes sequenced to completion-human, chimpanzee, and rhesus macaque (5-7). PRSSLY was first discovered on the mouse (8) and dog (9) available chromosomes (initially named DYNG in dog), and homologs were subsequently found on the Y chromosomes of bull (10) and pig (11). PRSSLY appears to encode a massive protein (2,667 amino acid residues in dog compared to the ˜400 residue average for mammalian proteins). However, PRSSLY contains only one identifiable domain-trypsin-like serine protease-making it part of the large PRSS gene family, which consists of 27 autosomal family members in human. PRSSLY's expression pattern is testis-specific, suggesting a role in sperm development. Given its distinctive species distribution (conserved in diverse mammals, but lost in primates, felines, etc.), gene structure, and expression pattern, Applicants explored the function and evolution of PRSSLY, finding it to be a candidate for the first ancient, broadly conserved, meiotic driver.

### Results

### Sex Ratio of PRSSLY-Knockout Offspring is Skewed Towards Females

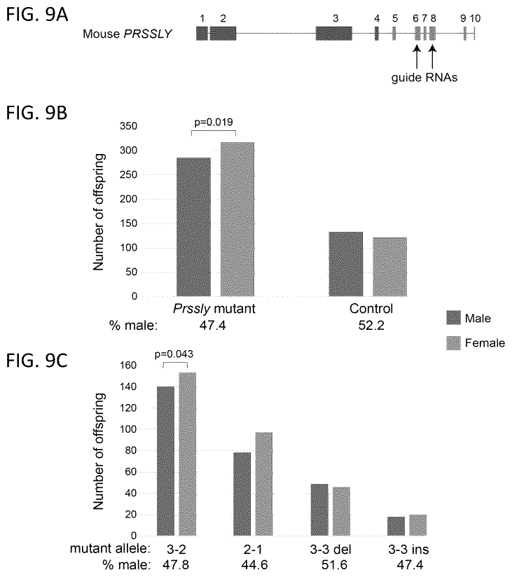

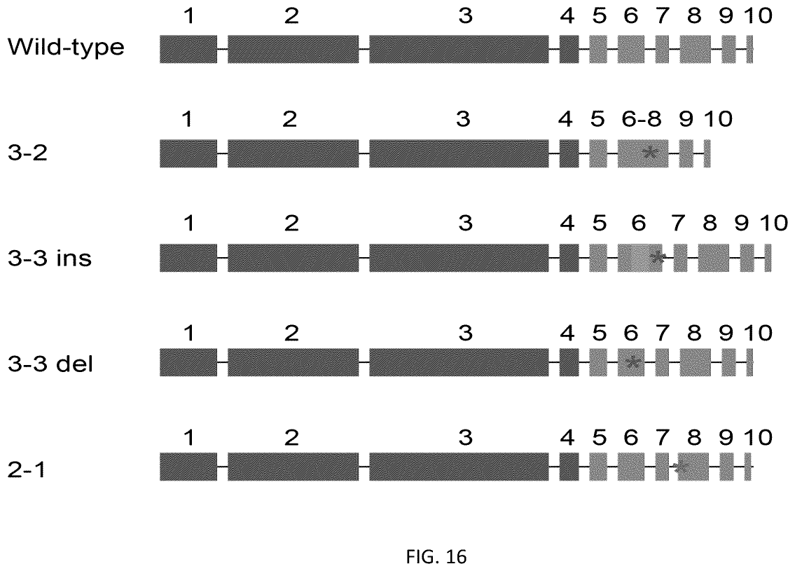

Applicants explored PRSSLY's function by generating CRISPR knockout mutations in mice. Applicants designed guide RNAs to target exons 6 and 8, which are part of the conserved trypsin-like serine protease domain (FIG. 9A). Applicants obtained four founder males with various frame-disrupting mutations: 3-2 contains a 407-bp deletion between exons 6 and 8, creating a premature stop; 3-3ins contains a 289-bp retroviral insertion into exon 6, creating a premature stop; 3-3del contains a 14-bp deletion in exon 6, creating a premature stop; and 2-1 contains a 47-bp deletion, including the first 20 bp of exon 8, likely disrupting splicing (FIG. 16). Given PRSSLY's testis-specific expression pattern in mouse, dog, and bull (8-10), Applicants anticipated that these mutations might affect spermatogenesis. However, all mutants were fertile and had normal testis histology.

Applicants continued breeding the mutant lines and a clear phenotype gradually emerged: the sex ratio of the offspring of the PRSSLY mutant males was skewed towards female. Applicants generated a total of 95 litters and 601 mice. Among these 601 offspring of PRSSLY mutant males, 47.4% were male, which is significantly lower than the 52.2% males Applicants observed among 255 offspring of control males (FIG. 9B). If Applicants consider each of the four mutant lines separately, the strength of the sex-ratio skewing Applicants detected correlates with offspring number, with 2-1 and 3-2 having the strongest signal (FIG. 9C). These results imply that PRSSLY functions to maintain an equal sex ratio and may be involved in a meiotic drive conflict with a distinct yet unidentified locus, perhaps on the X chromosome.

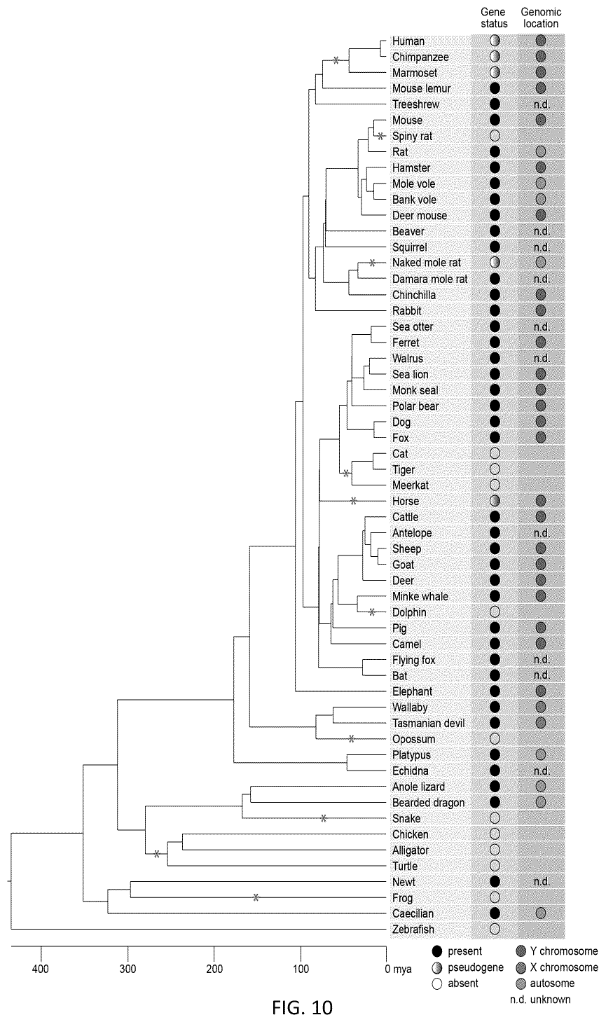

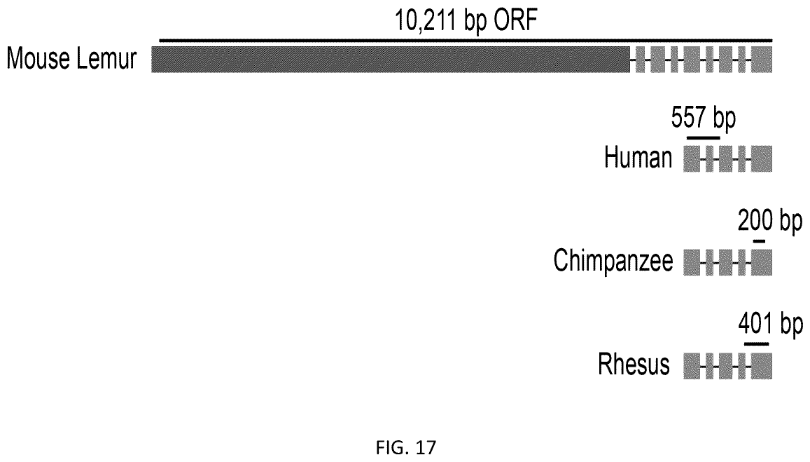

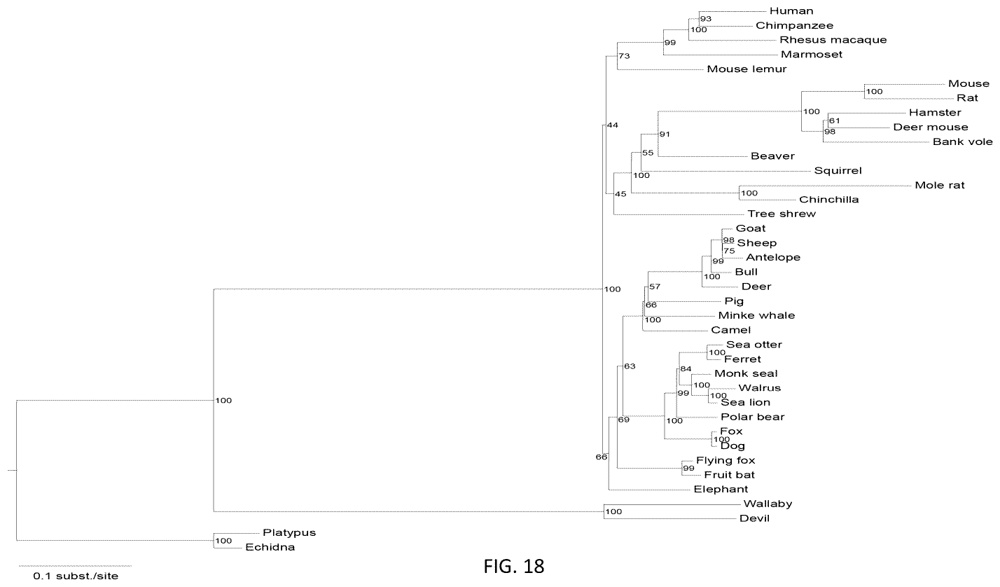

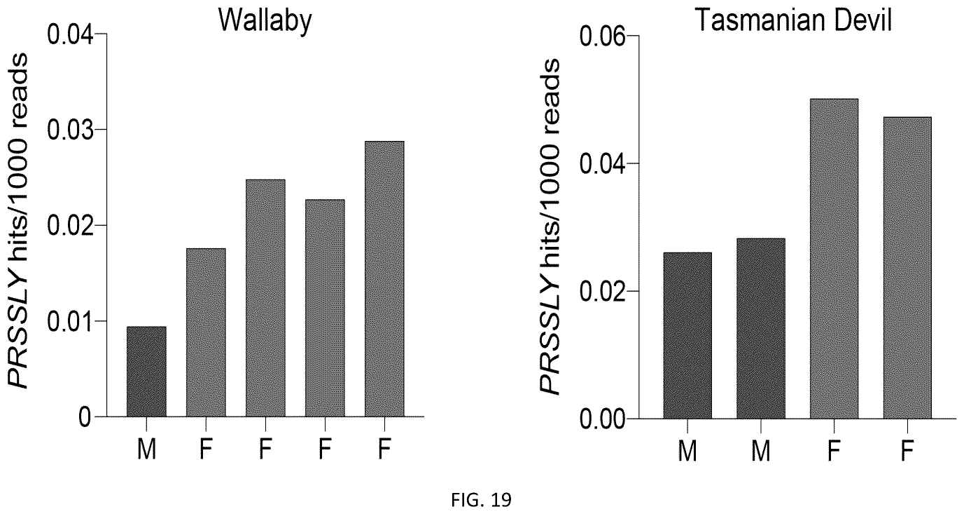

Next, Applicants set out to determine the conservation of PRSSLY in mammals by conducting a comprehensive survey of mammalian genomes for the presence or absence of PRSSLY. The number of complete Y chromosomes available for analysis is limited, so Applicants searched available male genomic DNA and testis RNA-seq datasets. In total, Applicants examined datasets from 47 mammals and found that PRSSLY is present in species representing every major mammalian lineage, but it has been lost or pseudogenized repeatedly in multiple lineages: primates, felines, naked mole rat, horse, dolphin, and opossum (FIG. 10). For 24 species, there is evidence of Y linkage because i) PRSSLY sequence was found in available Y chromosome sequence or ii) PRSSLY was present in male whole-genome-shotgun sequence or RNA-seq datasets but missing from available female whole-genome-shotgun sequence (Table 3). The human, chimpanzee, and rhesus Y chromosomes contain loci with homology to PRSSLY, but these loci are pseudogenes, with truncated open reading frames and no evidence for transcription in these species (FIG. 17). Y-linkage in mammals is not universal, however. PRSSLY has apparently translocated to an autosome at least three times in the rodent lineage (rat, vole, and naked mole rat) (FIG. 10). Phylogenetic analysis confirms that the autosomal copies in these species cluster with other PRSSLY homologs as expected, indicating that they are recently transposed (FIG. 18). PRSSLY homologs are X-linked in marsupials and autosomal in monotremes (FIG. 10 and FIG. 19). Beyond mammals, Applicants found PRSSLY homologs in species as divergent as lizards, newts, and caecilians (FIG. 10) so it likely arose in the tetrapod ancestor. However, this gene appears to have been lost in several major tetrapod lineages, including archelosauria (birds, crocodilians, and turtles), snakes, and frogs.

TABLE 3

| | Common | | GenBank accession |
|---|---|---|---|
| Species | name | Chromosome | number(s) |
| Microcebus | Mouse | Y | SRX670586, |
| murinus | lemur | | SRX270644 |
| Tupaia belangeri | Treeshrew | nd | NW_006200268, |
| chinensis | | | SRX157965 |
| Mus musculus | Mouse | Y | KJ780361, AC279166 |
| Rattus | Rat | 14 | MW769785, |
| norvegicus | | | SRX196307, |
| | | | SRX196316, |
| | | | SRX196298 |
| Mesocricetus | Golden | Y | SRX9824657 |
| auratus | hamster | | |
| Ellobius | Mole vole | autosome | SRX1466572 |
| lutescens | | | |
| Myodes | Bank vole | autosome | SRX2209239 |
| glareolus | | | |
| Peromyscus | Deer | Y | SRX2186902-3, |
| maniculatus | mouse | | SRX2186910-12, |
| | | | SRX2186914 |
| Neotoma lepida | Desert | nd | LZPO01017748.1 |
| | woodrat | | |
| Castor | Beaver | nd | NW_017873024, |
| canadensis | | | SRX2387506-9 |
| Urocitellus | Arctic | nd | NW_020540734 |
| parryii | ground | | |
| | squirrel | | |
| Fukomys | Damara | nd | NW_011045669, |
| damarensis | mole rat | | SRX347948, |
| | | | SRX347953 |
| Talpa | Spanish | nd | XM_037518333, |
| occidentalis | mole | | SRX4746078-9 |
| Chinchilla | Chinchilla | Y | SRX114649 |
| lanigera | | | |
| Oryctolagus | Rabbit | Y | SRX110711 |
| cuniculus | | | |
| Enhydra lutris | Sea otter | nd | NW_019154294 |
| kenyoni | | | |
| Mustela | Ferret | Y | NW_004577698, |
| putorius furo | | | SRX112813 |
| Mustela | Ermine | Y | XM_032331896.1 |
| erminea | | | |
| Odobenus | Walrus | nd | NW_004450708 |
| rosmarus | | | |
| Zalophus | Sea lion | Y | SRX4928796 |
| californianus | | | |
| Neomonachus | Hawaiian | Y | NW 018729642 |
| schauinslandi | monk seal | | |
| Ursus maritimus | Polar bear | Y | NW_007926558 |
| Canis lupus | Dog | Y | KP081776, |
| familiaris | | | SRX5279848 |
| Vulpes vulpes | Red fox | Y | SRX2834490 |
| Bos taurus | Cattle | Y | AC152072 |
| Pantholops | Tibetan | nd | NW 005810830 |
| hodgsonil | antelope | | |
| Ovis aries | Sheep | Y | SRX3274362, |
| | | | SRX3279754, |
| | | | SRX3279767 |
| Capra hircus | Goat | Y | SRX1947628, |
| | | | SRX1947629, |
| | | | SRX1947630 |
| Odocoileus | White- | Y | NW_018333206, |
| virginianus | tailed deer | | SRX2175795, |
| | | | SRX2175797 |
| Balaenoptera | Minke | Y | NW_006729412 |
| acutorostrata | whale | | |
| scammoni | | | |
| Sus scrofa | Pig | Y | MK393871, |
| | | | SRX229765-9 |
| Camelus ferus | Camel | Y | NW_006220272, |
| | | | SRX4610991 |
| Pteropus alecto | Black | nd | XM_025044285, |
| | flying fox | | NW_006433510 |
| Pteropus | Indian | nd | XM_039875739.1 |
| giganteus | flying fox | | |
| Rousettus | Egyptian | nd | NW_015493161, |
| aegyptiacus | fruit bat | | SRX1429054 |
| Loxodonta | Elephant | Y | SRX339470 |
| africana | | | |
| Macropus | Wallaby | X | ABQ0011031366, |
| eugenii | | | GL141582, |
| | | | CU234131, |
| | | | DRX012262 |
| Sarcophilus | Tasmanian | X | ERX779191, |
| harrisii | devil | | ERX779192, |
| | | | ERX779194, |
| | | | ERX779195, |
| | | | ERX2811915-8 |
| Vombatus | Wombat | nd | NW_020941101.1, |
| ursinis | | | ERX2730373, |
| | | | ERX2730372 |
| Ornithorhynchus | Platypus | 6 | SRX081891-2 |
| anatinus | | | |
| Tachyglossus | Echidna | nd | SRX317056, |
| aculeatus | | | SRX317058, |
| | | | SRX7214458 |
| Anolis | Anole lizard | 1 | SRX2704219-21, |
| carolinensis | | | SRX3367762, |
| | | | SRX3436885-6 |
| Pogona vitticeps | Bearded | 1 | ERX379375, |
| | dragon | | ERX697263 |
| Paroedura picta | Ocelot | nd | BDOT01000386.1 |
| | gecko | | |
| Zootoca vivpara | Viviparous | nd | XM_035097262 |
| | lizard | | |
| Lacerta agilis | Sand lizard | 16 | XM_033173881 |
| Podarcis muralis | Common | 17 | XM 028711624, |
| | wall lizard | | SRX5274936 |
| Cynops | Japanese | nd | SRX682040 |
| pyrrhogaster | fire belly | | |
| | newt | | |
| Rhinatrema | Two-lined | | NC_042615, |
| bivittatum | caecilian | 1 | SRX2848297 |
| Microcaecilia | Tiny | 2 | XM_030192074 |
| unicolor | cayenne | | |
| | caecilian | | |
| Geotrypetes | Gaboon | 1 | XM_033921891 |
| seraphini | caecilian | | |

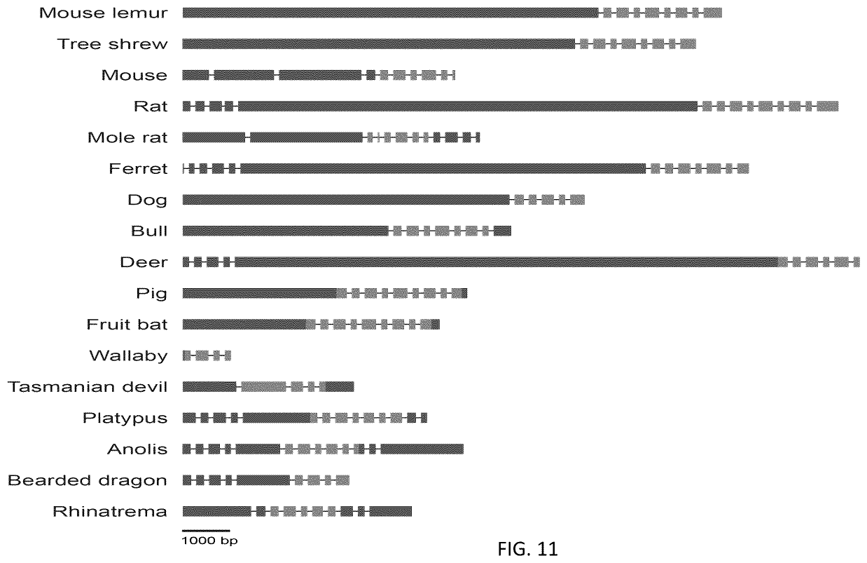

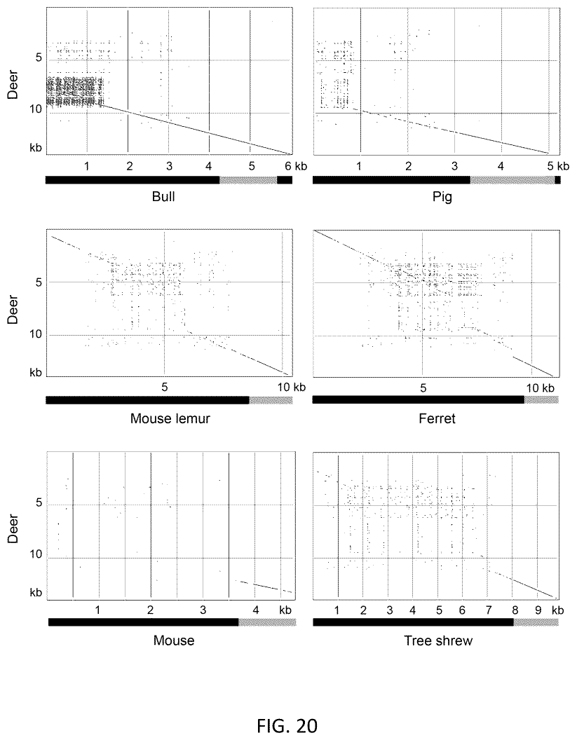

The unusual gene structure of PRSSLY differs vastly between species (FIG. 11). The region encoding the conserved trypsin-like serine protease domain is contained within four to nine exons spanning ˜1750 bp on average. However, in many species (e.g., mouse lemur, tree shrew, ferret, and deer), the entire open reading frame (ORFs) spans >10 kb, with the majority of the ORF residing in a single exon (FIG. 11). These massive exons dwarf typical exons, which are ˜300 bp on average in the human genome, and rival the longest known coding exon, which is ˜21 kb (in the gene MUC16) (12). These long ORFs show homology between species but, on average, are less conserved than the trypsin-like serine protease domain (FIG. 20). Considering all pairwise comparisons in mammals, the average percent nucleotide divergence is 24% within the conserved domain compared to 37% in the upstream region.

### PRSSLY is Testis-Specific in Eutherians but More Broadly Expressed in Other Animals

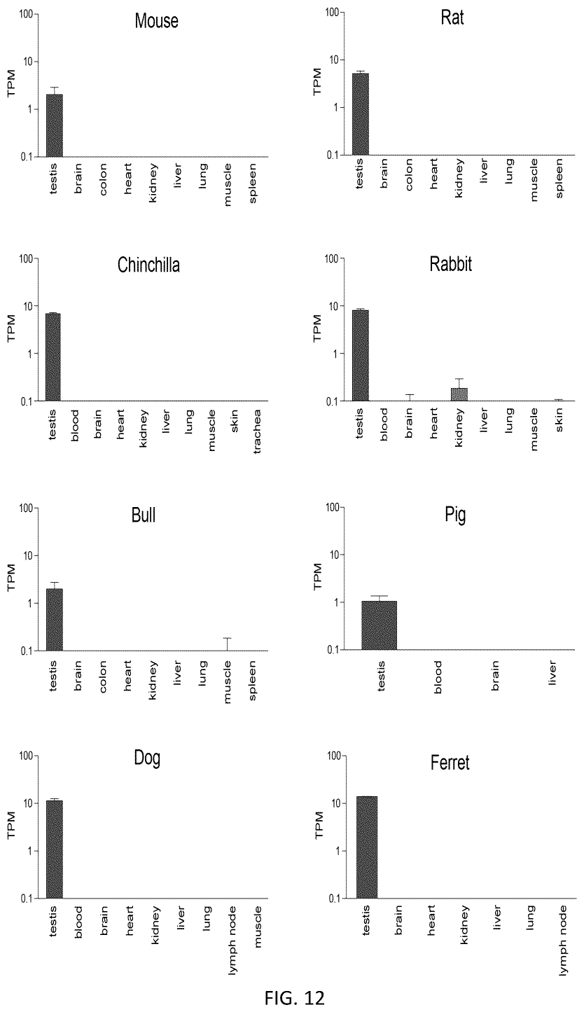

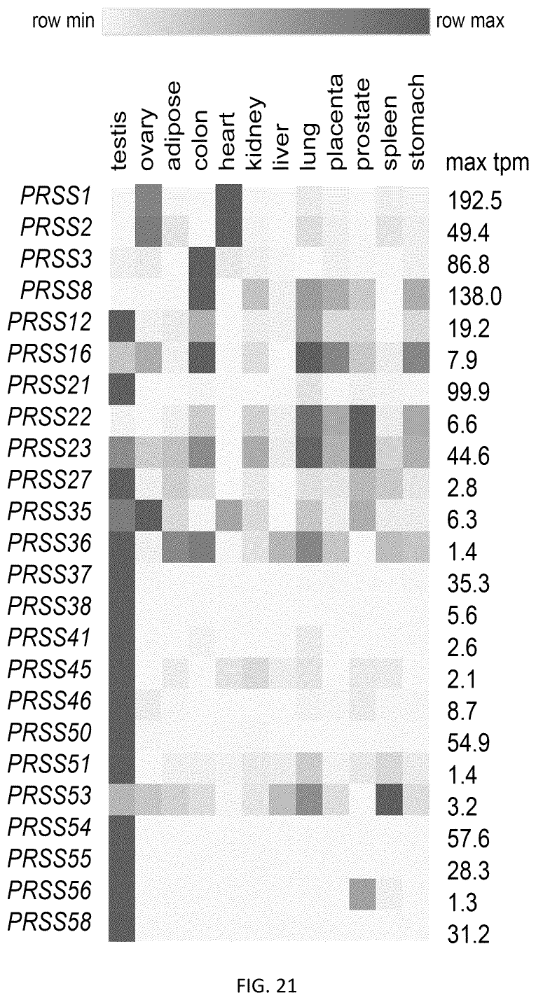

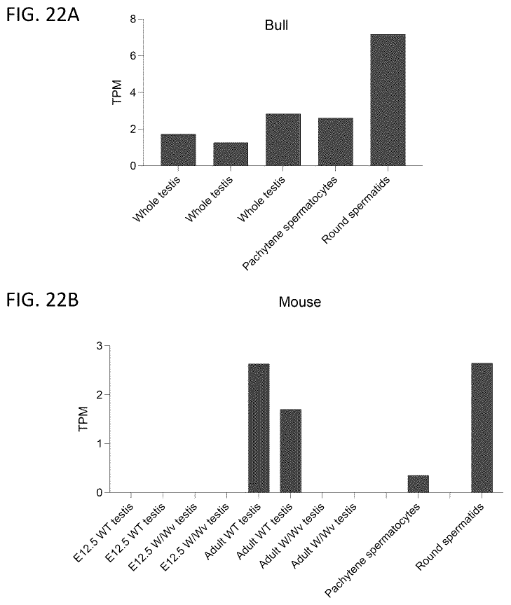

Next, Applicants investigated the expression pattern of PRSSLY and its homologs across species. Applicants examined expression in species where RNA-seq datasets from multiple tissues, including testis, were publicly available and the transcriptome was well annotated. In eutherian mammals, where Y linkage is nearly universal, PRSSLY is testis-specific (FIG. 12). This testis-specific expression pattern is also observed in about half of the 27 autosomal members of the PRSS gene family, including PRSSLY's closest relative PRSS55 (FIG. 21). Applicants were able to refine the expression pattern of PRSSLY in mouse and bull. Using a germ-cell depleted mouse model (13, 14), Applicants found that PRSSLY is expressed exclusively in adult germ cells (FIGS. 22A-22B). In bull, Applicants analyzed previously published RNA-seq datasets generated from purified germ cells (pachytene spermatocytes and round spermatids) (15), and were able to detect transcription of bull PRSSLY in these samples (FIGS. 22A-22B), providing evidence that it is transcribed in male germ cells.

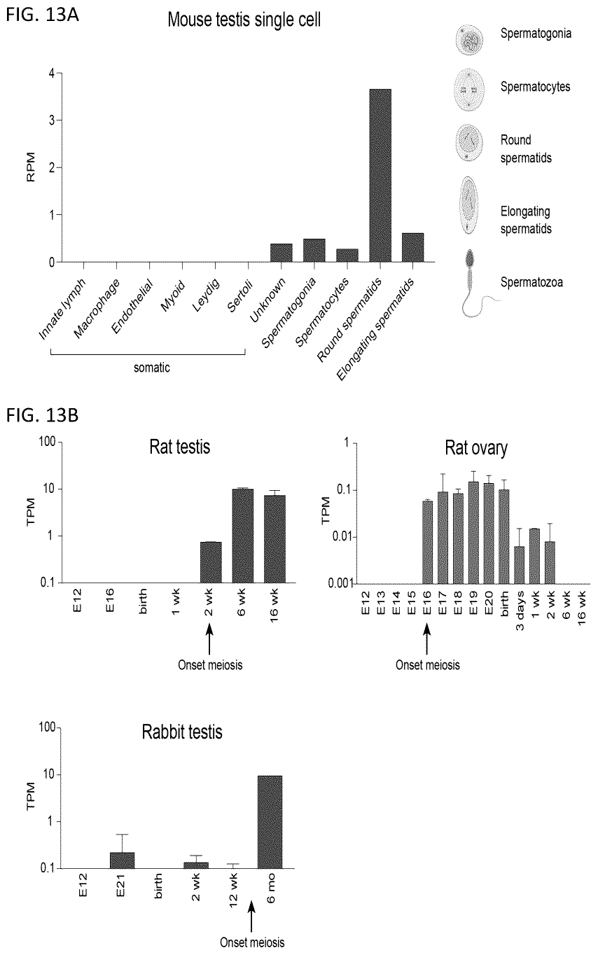

Next, Applicants examined the timing of PRSSLY expression in finer detail. For mouse, Applicants examined publicly available single-cell RNA-seq datasets generated from adult whole testis (16). Our analysis confirmed that PRSSLY is expressed only in germ cells and is absent in six somatic cell types included in this dataset (FIG. 13). In germ cells, PRSSLY is barely detectible in pre-meiotic and meiotic cells (spermatogonia and spermatocytes, respectively), but is highly expressed in round spermatids, which is the developmental stage immediately following meiosis (FIG. 13). PRSSLY expression is greatly reduced during the next stage of post-meiotic development (elongating spermatids).

Applicants also examined publicly available testis RNA-seq datasets covering multiple developmental timepoints from embryo to adult (17). Such datasets were available for rat and rabbit, and in both species the onset of PRSSLY expression correlates with the onset of meiosis (FIG. 13). In rat, where PRSSLY has transposed to an autosome, Applicants looked at available time course RNA-seq data from a variety of female tissues: ovary, brain, heart, kidney, and liver. PRSSLY expression was detected, at very low levels, in ovary, but was absent in all somatic tissues (FIG. 13). As in testis, the onset of PRSSLY expression in ovary correlates with the onset of meiosis, but the functional relevance of this ovarian expression is unknown. The factor that activates PRSSLY at the onset of meiosis may be expressed in both males and females and conserved between mouse and rat. Since the entire PRSSLY gene, including introns, was transposed from the Y chromosome to chr14, the promotor region was also likely transposed, accounting for the conserved expression patterns.

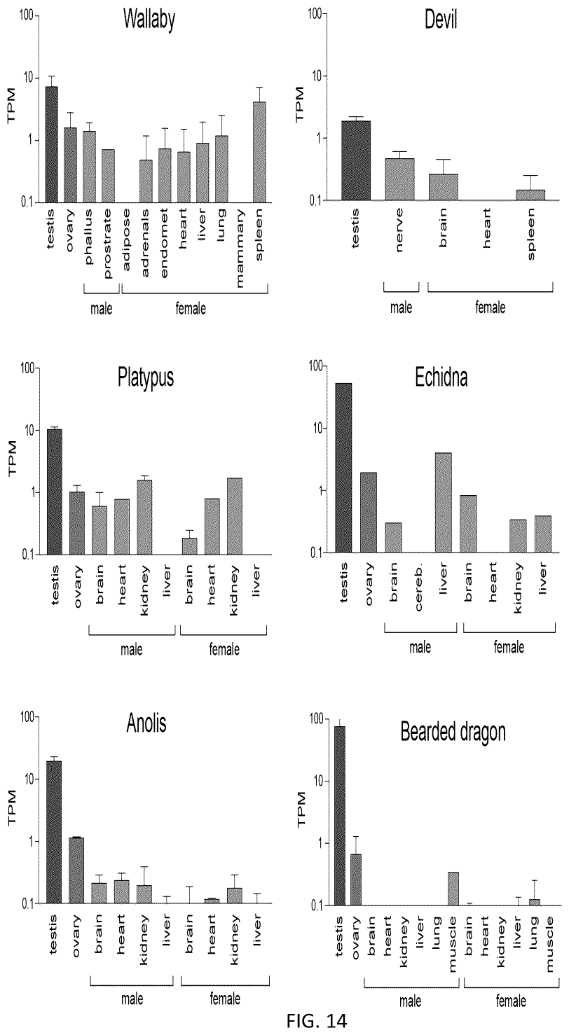

Outside of eutherians, PRSSLY homologs, which are located on the X chromosome or autosomes, are more broadly expressed (FIG. 14). Applicants examined publicly available RNA-seq data for two marsupials, two monotremes, and two lizards where multiple tissue types, including testis and ovary, were available. Non-Y-linked PRSSLY homologs are expressed in both males and females in both gonadal and somatic tissues. In most species, especially the lizards, PRSSLY expression is highest in testis.

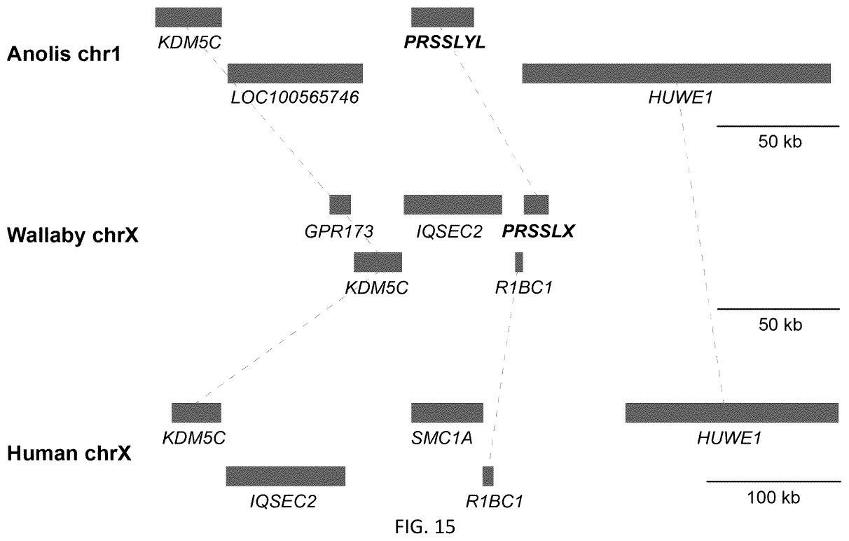

The chromosomal location and expression pattern of PRSSLY has evolved over time. The most parsimonious explanation for its evolutionary trajectory is supported by synteny analysis (FIG. 15). Applicants propose that the gene originated in the tetrapod ancestor on the autosome pair that eventually became the proto-X and Y chromosomes in mammals. After the proto-Y chromosome acquired the male-determining gene SRY, the sex chromosomes followed distinct evolutionary paths. PRSSLX/Y was lost from the Y chromosome but retained on the X in marsupials, and lost from the X chromosome but retained on the Y in eutherian mammals. The Y-linked version in eutherians then became restricted in its expression pattern, perhaps acquiring a novel function in spermatogenesis related to sex-ratio skewing. This evolutionary trajectory is highly unusual. While ˜90% of the 635 genes once shared between the X and Y chromosomes have been lost from the mammalian Y chromosome and retained on the X chromosome, PRSSLY is the first and only example of an ancestral X-Y pair gene lost from the X chromosome and retained on the Y chromosome.

### Discussion

Applicants characterized a novel testis-specific Y-linked gene—PRSSLY—that is widespread in eutherian mammals and has ancient origins, dating back at least ˜350 million years. Applicants found that PRSSLY knockout mice are fully fertile and appear to have normal spermatogenesis, yet produce more female offspring than expected, indicating that this gene is involved in meiotic drive. In mice, another sex-linked meiotic drive system exists: Sly-Slx/Slx1. Unlike PRSSLY, Sly-Slx/Slx1 are not conserved outside of the Mus lineage. Sly-Slx/Slx1 are also highly amplified on the sex chromosomes, with ˜120 copies of Sly on the mouse Y long arm and ˜40 copies of Slx/Slx1 on the mouse X chromosome. Mice with a deletion encompassing two-thirds of the Y long-arm produce excess females (38% male) (18). ShRNA-knockdowns of Slx/Slx1 in males results in offspring sex ratio skewing towards males (60% males) (19). A separate study showed that targeted deletion and duplication of the Slx/Slx1 gene family skewed sex ratios towards males and females, respectively (20). Another contrast with PRSSLY is that Sly and Slx/Slx1 deficiencies result in sperm head/spermatid elongation defects and sperm release defects, respectively (18, 21). Double knock-down of Sly and Slx/Slx1 rescues both the sperm defects and sex ratio (19).

Although Applicants have not identified an X-linked meiotic drive partner, Applicants surmise that PRSSLY may have arisen to suppress such a driver. X-linked drivers are much more common in nature than Y-linked drivers (2). Theory predicts that X drive and Y drive would initially have opposite effects on population size, increasing with X drive and decreasing with Y drive. Because the limiting factor for population growth is production of the egg, increasing the proportion of males via Y drive would quickly drive a population towards extinction (1). When an X-linked driver emerges, selection will strongly favor the evolution of a Y-linked or autosomal “suppressor” to restore a balanced sex ratio (3), and most characterized X drivers exist in equilibrium with a suppressor (2). Y-linked suppressors are more accurately described as “resistant” Y chromosomes, since X drive is thought to occur by impairing Y-bearing sperm (1). If a driver is lost through mutation, then selection to maintain the suppressor is eased, thus creating a cyclical arms race (22). PRSSLY's independent loss in multiple lineages is consistent with the instability of meiotic drive genes. Perhaps the unknown X-linked driver that PRSSLY confers resistance to was lost in primates, felines, etc., leading to loss of PRSSLY in those lineages.

Although Applicants do not yet know the mechanism by which PRSSLY balances the sex ratio, perhaps by promoting the survival of Y-bearing sperm, PRSSLY likely operates directly in the male germline at or after the onset of meiosis based on its expression pattern. The function of PRSSLY's closest relative—PRSS55—may also provide some clues. PRSS55 is essential for male mouse fertility, playing a role in sperm migration and sperm-egg binding (23) as well as structural differentiation and energy metabolism (24). Although PRSSLY is not required for fertility, it may act in a similar post-meiotic fashion to ensure the propagation of Y-bearing sperm. Indeed, the post-meiotic mechanisms of many X drivers are well characterized (25, 26), while the functions of suppressors of drive remain enigmatic.

This study uncovers the first putative meiotic drive system beyond rodents, insects, and plants. Unlike all other meiotic drive factors, PRSSLY is not lineage-specific, but has been conserved across tens of millions of years of evolution. Future investigations regarding PRSSLY's partner in meiotic drive and its precise function in spermatogenesis will help explain its unusual longevity. The identification of PRSSLY has practical applications as well, opening the door to the possibility of manipulating sex ratios in livestock, which would be of great interest, both biologically and commercially.

### Materials and Methods

### Identification of PRSSLY Homologs

Applicants performed tblastn searches of NCBI's non-redundant nucleotide database using PRSSLY sequences from bull and mouse as query sequences. Once more divergent PRSSLY sequences were identified (i.e., wallaby, lizard, and caecilian), Applicants repeated the tblastn searches with the newly identified sequences as queries. To search for PRSSLY in species without available male genomic sequence, Applicants scanned NCBI's Sequence Read Archive database for available testis RNA-seq datasets and performed mapping analyses using PRSSLY sequence from the closest relative species (See Table 3).

### Alignment, Phylogenetic, and Dot Plot Analyses

Nucleotide sequence alignment of conserved regions of PRSSLY homologs was performed using PRANK with default parameters (27). Phylogenetic tree using nucleotide alignment was generated using PhyML with default parameters (28). Dotplots were generated in Mac Vector using default parameters. Amino acid sequence alignment was performed using Muscle with default parameters (29).

### RNA-Seq Analysis

For each species, RNA-seq datasets were downloaded from NCBI's Sequence Read Archive database, and transcriptomes were downloaded from Ensembl. For bulk analyses, RNA-seq reads were mapped to their respective transcriptomes using Salmon with the mapping validation option enabled (30). For single-cell analysis, reads were mapped using Bowtie version 1.2.2 (31), and cell types were assigned as previously published (16).

### Generation of CRISPR Mutations

The Prssly knockout mice were generated via a CRISPR/Cas9-mediated strategy on the C57BL/6J background. Applicants designed two gRNAs, one targeting the end of exon 6 and the other targeting the start of exon 8, with the goal of producing a cut at both sites, and ideally, a deletion of the genomic DNA between these two sites. Experimental and control animals were backcrossed to C57BL/6J for an additional two generations or more. Deletions and insertions in founders and offspring were confirmed by PCR amplification and Sanger sequencing. Male offspring with edits to Prssly were subsequently backcrossed to C57BL/6J for two or more additional generations. The integrity of the long arm was confirmed by 18 PCR assays spanning the mouse Y. All experiments conformed to principles and guidelines approved by the Committee on Animal Care at the Massachusetts Institute of Technology.

### Sequences

Accession numbers for confirmed cDNA sequences:
Bull KC120771
Mouse KJ780361

Accession numbers for predicted genes:
Camel XM_006195429
Antelope XM_005964542
Flying fox XM_025044285
Polar bear XM_008710443
Minke whale XM_007188461
Tree shrew XM_006162722 (verified with RNAseq data; GenBank accession: SRX157965)
>Cattle (assembled from Y chromosome genomic sequence: accession number AC152072) SEQ ID NO: 11

AGAGACTGCCCCTTTCACAGTCCCAACATGGACTCTGGCTAAAAG
ACCAGCTGTAAAATCCTGGAGAGAGAATGTGCCTTTCACAACTCCACCATGGACA
TTGGCTGAAGGTCCACCTGTAAATACCTGGAGAGAAACTCTGCCTTTCATAGGCC
CACCTTGGACTCATGCTGAAAGTACAGCTATAAATAGTTGGAGAGGGACTGTGCC
ATTTGCAACTGCACTATGGGCTCTAGATAAAGGTCCAGCTGTAAATACCTGGAGA
GAGATGATGACTTTCACAGCCCCACCATGGACACAGGCTGAAGGTCCAGCTGTA
AATACATGGAGAGAGACTATTCCTTTTACAGTCCCACCATGGACTCAGGATGAAA
GTCCAGGTGTAATTACCTGGGGAGCAACTGTCCCTTTCAGAGTCCCATCAAGGAC
ACAGATTGAAAGTCCAGATATAAACACCTGGAGAGAGACTGCACCTTTCACAGC
CCCAGCATGGGCACAGGCTGAACCTCCAGCTGTAAATTCCTGGAGAGAGATTATG
CCTTTTTCAGTCCCACCATGGACACAGGATGAAAATCCAGGTGTAATTACCTGGG
GAGAGACTGTCACTTTCAGAGCCCCACCAAGGACACAGATTGAAAGTCCAGATG
TAAACACCTGGAGAGAGACTGTGCCTTTCACAGCACTACCATGGACACAGGCTG
AAGGTCCAGATGTAAATACCCAAAGAGATACTGTGCCTTTCACAGGTCTACCTTG
GACTCAGGCTGAAAGTACAGCTGTGCATACTGGGAGAGATATTGTACCTTTCACA
GCTCCACCATGGACTCAAGATAAAGGCCCAGCTTTAAATACCCGGACAGAGACT
GTACCTTTAACAGGCTCACCTTGGACTCAGGCTGAAAATCCAACTGTAAATACCT
GGAGAGATAAGATGCCTTTAACAGCCCCACCCTGGTCACAGGTTGAACGTCCAGC
TGTAAATACATGGAGAGAGACTGTGCCTTTCACAGCACCACCATGGACTCAGGCT
GAACGTCCTGCTATAAATAGCTGGAGAGAGACTATGCCTTTCTCAGCTCTACCAT
GGACTCAGGCTGAAAGTCCAGCTGTAAACACCTGGAGAGACACTATGCCTTTCAC
AGACCCATCATGGACTCAAGATAAAAGTCCAGCTGTAAATAGCTGGAGACAGAT
TTTGACTTTCCCAGCCCAACCTTGGCCACAGGCTGAAAGTATAGCTGCAAATGAC
TGGACACGGAACGCCCCTTTTACAGCTCCACTGTGGTCACACACTGAAAGTCCAG
CTGTAAATACCTGGACAGAGGCTATGCTTTTCACAGGCCCTCCATGGACTCAGGC
TGAAAATCCAGCTACACATACCTGGAAAGTGAACGTGCATTACAGAGGCCCACC
ATGGACTCAGTCTGACTCTGCACAAGCAAACCCTTGGACATCAACTGAAAGTTTT
AGAATCAGATCATGGACTCATGGAGTAAAGCAAGTTTTGAATATTTGGACAGAG
CCAATAGCTTCCACAGTTACACTTCGGACTCAGGCTGAATATTCAACACTAAAAT
ATTGGGCAGAGACTAAAGTCATTTACATAGTCACACCATTGACCCAGTGTCAGTT
TCCAATAAATACTTTGACAGAATCTGTAGGATCCATAATCACACCTTGGACATCT
GCTGAATCTCTAGTATTAAGTTCTTTCACACAGAATATTATTGATATAATCAAATT
TTGGCCAGTGCTTAAAACTGAGTCTAAGAAAAGGTGGAATCTGCCTCAAACTGAT
ACACTCATATTTTCACTAAATCCTCAAACTGATACTTTTGGATCCTTGAACCAAAT
TGAAAATCAAGAATCTCCTCTGTGGACACATCCTGAAATTGATAATGTCAATACA
ATGAACTTTCTTGAATCTGGAACACTCATATCACAGGTAGTATCTCTGCCCCAAG
CAGCTAGACTCTGGCCCCAAACTGAAGCTGATATTAGCAAAACTTGGTTTGTATC
CTCTGAAAGAATAAATTCTTGGGACCAATCAGAGTCTCAAAGAATGAGTACCTCA
ACCCATTTTGGAGTGGGTAGAGTAAAGCCCTTGGCCCAACATGAAACTGCTATAG
TCATGTCATGGCTTCAGATTGAAACTGGTATATTCTACCCTTGGAACCAGTCTGA
GGGAGACACAGTGAGGTTCTGGCCCCTTTCTGAAACTGAGGATGTAAGAGAATG
GATCCAAACTGGAGCCAGTAGAGTTAACTCTTGGACTCAACCGAGAACTAGTATA
GTCAGAGCTTGGCCCCAAGCTGAATCTGAACTAGTCAGACCCTGGACACAAACTA
AAACTAATGCAATCACACTATTGACCCAGGCTGATACTATCAAACCTTGGTTCCA
AACTCAAATTAATGCAATAAGAGAAGGAGCCCAAACTCAATCTCAAATTGTTACT
AGTATCCAAACACAGTTGCAAATAGTTAACCCCTGGATTCAGCCTAAAAGTGATT
CAATCAGATTTTGGACCCAGCCTTGGATCCAAGCTGAAACCCACACAGTCAGACT
CTTTTATGAAATTGATATAAGAAAATCATGGGCCTCATTTGGATCTCAGTCAGTC
ACATTTTGGTCACTGAGTCAAAATTCAGTTAGGACCTCATTTCACTTTGAATCTCA
GATGACATGTTCCTGGATCGGAAATGAATTTGATATAATCAGTCTTTGGAATCAA
TATGAAACTAGTTCAGTTGGATCCTGCATCAAGTCTGAAACTGGTACATGTCAAC
CCTGGGTCCATATTGAATCTTCTACAATCACACCATGGACCCAATATGAAACTTT
AGAGATCTACCCTTCAACCCAGCCTGAGACTGATACAGCATTAAGGCATTGGTTC
CAGCCCCAAATTGATCCAATTAATACTTGGAATCAGCCCGAAGTAGATACAATCA
GATTCTGGACCCAAGTTGAAACAGAAACAATTTCAGTTTGGACCCAGATTGGAAG
TCAAGTAGTTAAACTTTCCAACTTTTCTGAAGTTGGCATAGTTACACCTTGGCTAG
AGACTGAAACTGATACAAGTAGACCCTGGATTCAGTCTGACTTTCAGTCAGTCCA
TCCTTGGACCCAGACTGGATTTGGTATAATTAACCCCTGGTATCAGCCAAGAGCT
GCTGTAAATCAACCCTGGACATTTGTTCAAACACAGTCAATCGGACCCTGGACTA
AGGTGGAAGCCAATACAATCAAATCTTGGTTTCATGTTCAAATGAAAAAAGTCAG
ACTGGGGATTCCTGCTGAGTCTCAAATATTGAGTTTCTGGATGCAGTCTGATGTTA
GTAGAGTTAATGCTTGGATCCAACCAGAAACCCAGGCAGTCAATCCTGGGGCTCA
TCCTAAATCTGGTAATGTTGCATCCCTGGCTATTCCTAAGCCTGAAAGAGTCAGA
ATGTGGATCCAGCCTGAAACAGAAATAAGGCCTGGCATCATTTATAAAACTGATA
TAACCACATCATTTGCTTCTCCTGAAATTGAACCAGATGGAACAATTAGTCATTTT
GATTTCTTGTCTAATCGTGTAACATTTTTAACAATAGAAACTGTTCCTTCCCTAGA
TGAGCATTTTGCAGCTTTGTCAACTGAAATAGCTGCAGTAGAAAGCCAAGGTCAA
ATAAATTCTGTCCAACCCAGTGAGATCACAAATACTCTCTTTCTTACACTTTCAAG
CACATGGCTTCCTGGAGGAGCTGGTTACCTGAACTTTGCCAATAAATTGCAAATT
ACCAAAACAAAAGGAAGCCCTAATGTCCCATCTAGTTCTCTCAACCCACTTTTTC
CATCTTTTTCCTTTCCTGTTCCTTGTTTTATCCCATTTTCATGTTCTTTGTCCCTTAC
TTGTTCAGTCTTTTCTTCTTGCACACTTTCTTCACCATGTACTTTTCCTTCTTGCTCA
GTTCTTCCTCTTGTGGCTTTCTCTCCTGTTCTTCCCTTAGCTGCTTCTGATAGTTCT
CTCCAGAAACCATCTTCCTCAGAAGTTACTGAAGACACCATTCTTTCCCATACTTT
TTCATCCTTGCATGCTGCTCCAGCCACTCTTTTAACAAAGCAACCATCTCTGATGC
CTGGATTTCAATCTGAAACCAAGTCTAATCGGCCTGAACAAGATCTTCCTAAGTA
TTCTGAACTCAATGTTTCCCTTGCTGAGTGTCGCCTGGATGTGGTCTGGAAAGAG
AGTCTCCAGGCTTTCTCGCTCTTCAAGACAGCTGTTATTTCTCATGAAATCACAGA
GTGTGGATTACGCCCTGGCCTTGTTCCACACTGCCCCAACTGCTGGGAGGCTGAA
GTGGGTGAATTCCCTTGGATGGTTTCTGTGCAACTCTCTTTCTCCCATTTCTGTGCT
GGTTCTATACTGAATGAACAGTGGATTCTCACTACAGCTAGATGTGCAAATTTCA
TAAAAAACTCAGAAGCATTGGCCCATGTCCAGGTGGGGCTGATTGATCTTCAAGA
CCCTGCTCAAGCTCAAACTGTAGGCATTCATCGTGCCATGCCCTACCTGGGCCCT
AGAGGACCTTTGGGACCTGGTCTAATCTTCTTGAAGCAACCATTACATTTTCAAC
CCCTGGTTCTTCCTATCTGCCTGGAGGAGAACCTAGAGCAAGAGAAAAATATACA
ACTATATGACTGCTGGCTACCTAGTTGGTCCCTCATGAGAGGAAGTCCTGGAATT
TTGCAAAAAAGGCATCTGAGCATCCTGCAAGTCATCACATGTGCCCAGTTTTGGC
CCAGCCTGAATGAATTTACTTTCTGTGTGGCAGCCAAGAAAGCTATGGGGGAGGC
TGGCTGTAAGGGTGACCTGGGGGCACCTCTTATATGTCATCTGCAACAAAAAGAC
ACATGGGTGCAGGTGGGAATCTTGACTCACTTTGATGAACACTGCAGAAAGCCCT
ATGTCTTCAGCCAAGTGAGCCCTTTCCTTTTCTGGCTCCAGGGAGTTACACGACCC
AGCCAAGCACCCTGGTCCAAGCAAGGGCCCATGACCACCTCTGCTTCCATCTCCC
TTTCAGTCTCCACCTCTATGAATGCCTCAGCTTTTACCTCCACACCTGCTTCTGTCC
GGCCACATTTCATCTCTCTGCCACAGCCTCAGACTTTGGCAGATCGAATTTCTCTG
AGATATGCCATGCCTTGGCAGGCCATGATCATCAGTTGTGGCAGTCAAATTTGCA
GTGGTTCCATTGTTAGCAGCTCTTGGGTACTCACCGCGGCTCACTGTGTCAGGAA
TATGAATCCTGAAGACACAGCTGTAATATTGGGCCTGAGGCACCCTGGGGCACCT
CTGAGAGTTGTTAAGATCTCTACCATTCTTCTGCATGAAAGATTTCGATTGGTGAG
TAGGGCAGCAAGAAACGATCTAGCATTGCTGCTCCTTCAAGAGGTCCAGACTCCC
ATTCAGCTTTTAGCACCGTTGGGTCATCTGAAGAACCTGAACAGCTCAGAATGCT
GGCTGTCTGGGCCAAGAATTCTTAAGCCAGGAGAGACAGATGAAAATCCAGAAA
TATTACAGATGCAGGTGATAGGAGCTTCAAGCTGTGCCCACCTATACCCTGATAT
AGGTAGTTCCATTGTCTGCTTCATTACACAAGACAAAGATTCTGACACAAGTGTG
GAACCAGTGAGTCCAGGCAGTGCTGTCATGTGCAGACCAATCTCTAGGAATGGA
AGCTGGAGACAGATAGGCCTCACTAGTCTGAAGGCACTGGCTACCATTGTGAGCC
CCCACTTCTCATGGATATTATCCACTTCATCAAAAGCAGGGCATCCATTAAGCCA
TGAACTAATGCCTTGGATGGAAAAGCCTAAGTCCTCTAGTCTCATAAAACAGCCA
GCCACCCTGCCATTTTATTCAATAATAATTGTTATACTACAAAAGCTTTCATAACT
CACTGTGAAAATAAGGCAGGGCTAATCTATTCAAACTATTTATAATAAAAATTTT
AAACAACATTAAAGAAAATTAAGACCCTATGCAACCTAGGAG

>Pig (assembled from Y chromosome sequence and testis RNA-seq: accession numbers MK393871. SRX229765-9) (SEQ ID NO: 12)

ctcagagaaaaatcttggctgcagtctgattttgaaaaattctcaccttCAACCCAGGTTCAATTT
GTATTGGTAAAACCCTGGACACAGTCTGAAAATGATACACTCATGCCATGGACCC
AGGTTCAACCTTCTGCAGTAAATGTCTGGACAAAGGCTACAGCTTTCACAGTCAT
ACCATGGACTCAAGATGAATTTTTTGCAGAAAAGTCCTGGACAGAGACTGTGGTT
TCCATAGTCACACTGTGGATTAAGGCTACATCTCCAGAAGTAAATACCTGGACAG
AGGTTTCTCTTTCCACAGTCACACCAGGGTCTCAAACTGGATCTCCAGAGGTAAA
ACCCTGGGCAGAGGCTCTAACTTCCACAGTTATACCCACACTACTGCCTCAGGAT
GAAATTTCTGCAACAAAATCCTGGACAAAGAATGTGGGTGCCAGAGTCACACTA
TGGACTCAGCCTGAATCTCCATCTGTCCATCCCTGGGCACAGACTGTAGCTTCCA
CAGTCACACTCACACCATGGACTCAGGATGAATTTTCTGTAACAAAATTCTGGAC
AGAGACTTTTTTTTCCACAGTTCCACAGTGGACTCAGGCTCAATCTCCCTTAAATC
CTTTGACAGAGACTATAGATGCCATAGTCACACCATGGATTCAGGCTGAATCTCC
AGGAGTAAATCATTTGACAGAGGCTGTTTCTTCCATAGTCACACCTACACCATGG
ACTCAGGCTAAAGATTCAGCAATAAAACCCTGGACACAGACTGTCACACCATGG
ATTCACACTCAATCTCCAGAAGTAAATCCCTGGACACAGACTGCAGCTTCCACAG
CTGCATTGTGGACTCATATTGAATCTCTAGCATTAAGTTATTCCGCACAGAGTATT
ACTGATACAGTTATATTTTGGACCATGCTTCAAACTGAATCTAAGAAACCCTGGA
GTCTTTCCAAACCTAGTTTATTCACTATTTCACTGAATACTCAAAGTGATCTTACT
GGATCACTGATTCATAATGAAAATCAAGCATATCTTCTGTTGACACATTCTGAAA
TTTATAATGTCAAGACACTGAACTTGCCTAAATCTGAAACACTCATATCATGGAC
AGTGCCCTTTCCTCAAGCAGTCAGTCTCTGGCCCCAATCTGAAGTTGATATTACCA
GAACTTGGTTTAAAATACCAGAAAGAAAAACATCCTGGGCCCAGTCAGAATCTCT
GACAATGAGTACCATGACACAGATAGGAGATGCTGGAATGAGGATGATAGCACA
GCATGAACCTGCTGAAGTCACATCATGGATTCAGACTGAAAGTGGTATATTCCAC
CCTTGGGAGAAGTCTGAAGGAGATATAGTGAGATTCTGGACCCCTTCTGAAACTA
AGCCTGTAAAACCATGGATGCAAACTGGAACTGCTTTAGTCACTCCACTGACCCA
GCCTGAAATGCAAGCATTAAAATACATGAGCATGCCTGACATTAATTCTATCAGA
CCTTGGTTCAAGACTCAAACTGAGCCTATAAGAGAAGGCACTCAACCTGAATCTC
AAATAGTTTCTACTAGGATCCAACCAGAACCACAAATCATCCATCCATGGTTCCA
GCCTGAAAGTAATGCAGTCAGATTCTGGACTCAGCCTAAAGGTAATTTAGCCCAA
TCCTGGTTTCAAGCTAAAGCCAATATAGTAAGATTCCAGACTCAGTATGAAATTG
ATACAGGAAAACCATGGACCCAGCCTGAATCTCAGTCAGTCACATTTTTGTCCTT
AACGCAAAATGATGCAATTAGTCCCACATCCCACTTTGAATCTCAGAAGACGTGT
TCCTGGACCCAAAATGAATTTGGTATAATCAGTCTTTGGACTCAGTCTGAAACTA
GTTCAGTCATATTTGGAACCAAGCTTGAAACTATTACAGGCCAACCCTGGCTTCA
TCTTGAATCTGCTACATGCAGACCATGGACCCTGCATGAAACCTTAACCCAGCCT
GAAGCTGCTACAGTAAGACACTGGCTCCATACCCAAATGGACTTAAAAGAACCTT
GGAATCAGCCTGAAGATGATGCAGTTATACACTGGACCCAGCTTGAAACAGAAA
CAATTTATGTTTGGACCCAGACTGAAAGAGAAGTAGTAAAACCTCCAACTTTATC
TGAAGTCAATTTAGTCACATCTTGGTTACAGACTCAAAGTGATATAATTAGACCT
TGGATTAAACCTGAGTTTCAGTCAGTCATTCCTTCGATCCACACTGGAGTTGATAT
ACTTCACTTCTGGTCTCAGCAAAGAGCTGCTACAAATCAACCCAGAACATACCCC
AAAACCCAGGTAGTCAGACCATGGATCAAGCTGGAGGCTGATACAATCAAATCT
TGGTTCCACATTCAAAGGAATAAAGTCAGACCATGGATGCCTACAAAATCTCAAA
TATTGAGCCTCTGGAGGCAGCATGAAGTTGATATAGTCCATCCATGGATCCAGCC
AGAAACCCTGGCAGACAGACCCTGGTCCCACTCTGAAACTGAGATTGCATTCTTG
GAAAGTTATAAACCTGACCAAGTTAGAACATGGATCCAGCCTGAAATAGAAATA
AGGCCTGGCATCCATTATAAAGCTGATAAAATGACATCATTTACTTCTCCTGAAG
TAGAGCAAAATGAAACAACCCAATTAACCAGTCACTTTGGCTCTTGGTATAAACA
TAAACCTTTTATACCAACAGAAAGTATTCCTTCCCCGGATGAGTATTTTACAGCTT
TGTCAACTGAGATAACTACACAAGAAATCCAAGATCAAATCAATTCTGTCCAACC
CACTGAGCTCACAGATATTCTCTTTTTCACCCTTTCAAGCATGTGGTTTCCTGAAG
GAGTTGGTTATCTAAAATTTGGCAGTAAATTACAAATTACCAAAACAAAAGGAA
GCCCTGAGTTCCCATCTACTTCTCAGAGCCCCCTTTCTCCATCCCTTTCCTTTCCTG
TTCCATGTTTTTCACCATCCCTGTGTTCCTTGTCCCTTTCCTGTTCGGTGTTTTCTTC
TTGCGCATTCCCTAGACCCTGTATTTTCCCTTCTTGCTCAAGTCTTTCCTCTGTGGT
ATTCTCTCCTATTCTCCTTCCCTTAGCTGCTTCTGATAGTTTTCTCCAGAAACTATA
TTACTCAAAAGTTACTGAAGAAACCATTCTTTCTCATACTTCTCCATCCCTGAATA
TTGCTTCAGCCATACTTTTAACAAAGCAGCCTTCCCTGATGTCTGGATCTCAATCT
GGAACCAAGTCTAATCAACCTGAACAAGATCCTCTCAAGTATTCTGAACTCAATA
TTTCCTTGGCTGAATGTCACCTGGGTGTGGTCTGGAAAGAGAATCTCCAAGCTTT
CTGGCTATTCAAAACAGCTGTTATTTCCCATGAAACCACAGAGTGTGGATTACGC
CCTGGCCTCGTCCCACATTGTCCCAACTGCTGGGAAGCAGAAGTGGGTGAATTCC
CTTGGATGGTTTCTGTGCAACTGTCTTTCTCTCATTTCTGTGCTGGCTCCATACTGA
ATGAACAGTGGATCCTTACTACCGCTAGATGCGCAAATTTCATAAAAAACTCAGA
AGCACTGGCCCTTGTCCAGGTGGGGCTTATTGATCTTCAAGACACTGCCCAAGCT
CAAACAGTAGGCATTCATCGTGCCATGCCCTACCTAGGTCCTAAGGGCCCTTTGG
GACCTGGGCTGATCTTCCTTAAGCAGCCATTACATTTTCAACCTCTGGTGCTTCCT
ATCTGCCTGGAAGAGAGTCTGGAGCAAGAGAAAAACATACAACTGTATGACTGC
TGGCTACCTAGTTGGTCTCTCATGAGAGGAAGTCCTGGAATTCTGCAAAAAAGAC
ACCTAAGCATCCTGCAAGTCAGCACGTGTGCTCAATTTTGGCCCAATTTGAGTGA
ATTTACTTTCTGTGTAGAAGCCAAGAAAGCTATGGGGGAAGCTGGCTGTAAGGGT
GACCTGGGGGCACCTTTGGTGTGCCATCTACAACAAAAGGACACATGGGTGCAG
GTGGGAATATTGAGTCATTTTGATGAACATTGCACAAAGCCCTACGTCTTCAGCC
AAGTGAGCCCTTTCCTTTTCTGGCTCCAGGGAGTTACACGACCAAGCCATGCTCC
CTGGTCCAAGCAAGGGCCCATGACTACTTCTGCTTCCATCTCACTTTCAGTATCTA
CCTCTACGAATGCTTCAGCTTTTACCTCCACTCCTGCTTCTGTCCAGCCTCACTTC
ATCTCACTGCCACAGCCTCAGACTCTGGCAGATCGGGTTTCTCTAAGATATGCCA
TGCCTTGGCAGGCCATGATTATCAGCTGTGGCAGTCAAATTTGCAGTGGTTCCAT
TGTTAGCAGCTCCTGGGTACTCACTGCTGCTCACTGTGTCAGGAACATGAATCCT
GAAGACACTGCTGTAATACTGGGCCTGAGACACCCAGGGGCACCTCTGAGAGTT
GTCAAGGTATCCACCATTTTACTGCATGAGAGATTCCGGTTGGTAAGTGGAGCAG
CAAGAAATGACCTAGCATTGCTGCTGCTTCAAGAGGTACAGACTCCCATTCAGCT
TTTAGCACCCTTAGGCCATCTAAAGAACTTGAATAGCTCAGAATGCTGGCTCTCA
GGACCACGGATTCTAAAGCCAGGAGAGACAGATGAAAATCCAGAAATACTGCAG
ATGCAGGTGATAGGAGCTTCAAGCTGTGCCCACCTCTATCCAGACATAGGCAGTT
CTATTGTGTGCTTCATTACACAGGACAAAGACTCCAATACAAATGTGGAACCGGT
GAGCCCAGGCAGTGCTGTTATGTGCCGACCAATGTCTGGGAATGGAAGCTGGAG
ACAGATAGGGCTCACCAGTCTGAAAGCTCTAGCTACCATTGTGAGACCCCACTTC
TCCTGGATATTATCCACTTCTGCAAAAGCAGGGCATCCCCTAAACCAAGCACTCA
TGCCTTGGGTAGAAAAGGCCAAATCCTCTAGTCTCTTAAAAAAGCCAACCATATT
ACCATCAGTAATAATTATTGCAATACAAAGCCTTTTGTAAGTTAATAGCTATAGA
GGGAGGAGATAATCTGTTCACACTGTACTGGAAAGTATATTAACAATGTCATAAC
AAGACTGATACATTTAGATAGAACTCAGTAATTCTGAAATAAAGAAATACTCTAT
CTAAATGACTAATTTCTTTTAAAACACTTAAAAAACAAATTAT

>Sheep (assembled from testis RNA-seq: accession numbers SRX3274362. SRX3279754. SRX3279767) (SEQ ID NO: 13)

CTCAGGATGACAGTCCAGGTGTAATTACCTGGGGAGAGACTGTTC
CTTTCAGAACCACATCAAGGACACCGATTGAAAGTCCAGATATAAACACCTGGA
GAGAGACTGTGCCTTTCACAGCCTCACAATGGACTCAGGCTGAACCTCCAGCTAT
AAATTCTTGGAGAGAGATGATACCTTTTTCCATCCCACCATGGACACAGGATGGA
AGTCCAGGTGTAATTACCTGGGGAGAGACTGTCCCTTTCAGAGCCCCACCAAGGA
CGCAAATTGAAAGTCCAGATGTAAACACCTGGAGAGAGATTGTGCCTTTCACAGC
ACTACCATGGACACAGGCTGAAGGTCCAGATGTAAATACCCAGAGAGATACTGT
GCCTTTTACAGGTCGGACTCAGGCTGAAAGTACAGCTGTGCATACTGGGAGAGAT
ACTGTGCCTTTCACAGCTCCACCATGGACTCAAGATAAAGGCCCAGATGTAAATA
ACAGCAGTGAGGTGCTGAGTTTCACAGGACCATCATTGGCACATGCTAAAGGTCC
AGCTTTAAATAACTGGAGGGAGACTGTACCTATAATAGGCTCATCCTGGACTCAG
GCTGAAAATCCAACTGTAAATGCCTGGAGAGAGAATATGCCTTTAACAGCCCCAC
CCTGGTCACAGGTTGAACATCCAGCTATAAATACATGGAGAGAAACTGTGCCTTT
CACAGAGCCACCATGGACTCAGGCTGAATATCCTGCTGTAAACACCTGGAGAGA
AACTGTGCCTTTCTCAGCTCTACCATGGACTCAGGCTGAAAGTCCAGCTGTAAAC
ACCTGGAGAGAGGCTATGCCTTTCACAGACCCATCATGGATTCAAGAGAAAAGT
CTAACTGTAAATAGCTGGAGACAGATTTTTACTTTCCCAGCCCAACCATGGCCAC
AGACTGAAAGTACAGTGGAAAACGACTGGATATGGAATGCCCCTTTTACAGCTCC
ACCATGGTCACACACTGAAAGTTCAGCTGTAAATACCTGGACTGAGCCTATGCTT
TTTATAGCCCCTCCATGGACTGAGGCTGAAAATCCAGCTACAAATACCTGGAAAG
TGAATATGCATTACAGAGACACACTATGGCCTCAGTCTGACTTTGCACCAGCAAA
CCCTTGGACATCAACTGAAAGTTTCAGAATCACCTCATGGACTCATACTGTAAAG
CAAGTTTTAAATATTTGGACAGAGCCAATAGCTTCCACAGCCACACTGTGGACTC
AGGCTGAATATTCAACACCAACATATTGGACAGAGATTAAGGCCATTTATATAGT
CACACCATTGACCCAGTATCAGTTTTCAATAAATACTTTGACAAAATCTGTAGGA
GCCATAATCACACTTTGGACGTCTGCTGAATCTCTGTCATTAAGTTCTTTCACACA
GAATAGTATCGATACAATCGAATTTTGGCCAATGCTTAAAACTGAGTCTAAGAAA
AGGTGGAATCTGCCTCAAACTAGTACATTCATGTTTTCACTAAATCCTCAAATTG
ATACTTTTGGATCCTTGAACCAAATTGAAAATCAAGAATCTCCTCGGTGGGCCCA
TCCTGAGATTGATAATGCCAATACAATGGCCTTTCTTGAATTTGGAACACTCATAT
CACAGGTAGTACCTTTGCCCCAAGCAGCTAGACTCTGGCCCCAAACTGAAGCTGC
TAATAGCAAAATTTGGTTTGTATCCTCTGAAAGAATAAATTCCTGGGACCAATCA
GAGTCTCAAAGAATGAGTACCTCAATCCATTTTGGAGTGGGTAGAGTGAAGCCCC
TGGCCCAACATGAAACTGCTATAGTCATGTCATGGCTTCAGATTGAAACTGGTAT
ATTCCACCCTTGGAACAAGTCTGAAGGAGGCACAGGGAGGTTCTGGCCCCTTTCT
GAAACTGAGGATATAAGAGAATGGATCCAAACTGGAGCCAGTACAGTTAACTCT
TGGACTCAACTGAGAACTAATATAGTCAGAGCTTGTCCCCAAGCTGAATCTGAAT
TAGTCAGACCCTGGACACAAGCTAAAACTAATGCAATCACACTATTGACCCAGAC
TGATGCTATCAAACCTTGGTTCCAAACTAAAATTAATTCACTAAGAGAAGGGACC
CAAACTCAATCTCAAATTGTTACTACTTGGATCCAAACACAGTTGCAAATATTTC
ACCCCTGGATTCAGCCTAAAAGTGATTCAGTCAGATTTTGGACCCAGCCTTGGAT
CCAAGCTGAAACCCACACAGTCAGACTCTATTATGAAATTGATGTAAGAAAATCA
TGGGCTTCATCTGAATCTCAGTCAGTCACATTTTGGTCACTGAGTCAAAATTCAGT
TAGGACCTCATTTCACTTTGAATCTCAGATGACATGTTCCTGGGTCCGAAATGAA
TTTGATATAATCAGTCCTTGGAATCAATATGAAACTAGTTCTGTTGGATCCTGGAT
CCAGTCTGAAACTGGTACATGTCAACCCTGGTTCCATATTGAATCTTCTACAATCA
CACCATGGACCCAATATGAAACTTTAGAGATCTCCCCTTCAACCCATCCTGAGAC
TGATACAGCAATAAGGCATTTGTTCCAGCCCCAAATTGATCCAATTAGTACTTGG
AATCAGCCTGAGGTAGATACAATCAGATTCTGGACCCAAGTTGAAACAGAAACA
ATTCCAATTTGGATCCAGATTGGAAGTCAAGTAGTTAAACCTCCCAACTTTTCTG
AAGTTGGTATAGTTACACCTTGGCTAAAGACTGAAACTGATGCAATTATTCCCTG
GATTCAGTCTGACTTTCAGTCAATCCATCCTTCGACCCAGACTGGATTTGGTATAA
TTGACCCCTGGTTTCAGCCAAGAGCTTCTGTAAATCAACCCTGGACCTTTGTTCAA
ACACAGTCAATCAGACCTTGGATTAAAGTGGAATCCAATACAATCAAATCTTGGT
TTCATGTTCCAATGAAAAAAGTCAGACTGGGGATTCCTTCTGAGTCTCAAATATT
GAGTTTCTGGTTGCAGTCTGATGTTAGTAGAGTTAATGCTTGGATCCAACCAGAA
ACCCAGGCAGTCAATCCTGGGGCTCATCCTAAAACTGGTAATGTTGCATCCCTGA
CTATTCCTAAGCCTGAAAGAGTCAGAATGTGGATCCAGCCTGAAAGAGAAATGA
GACCTGGCATCATTTATAAAACTAATATAACCACATCATTTGCTTCTGAAATTGA
ACCAGATGGAACAATTAGTCATTTTGATTCCTGGGCTAACCATGTAACATTTTTAC
CAATAGAAACTGTTCCTTCCCTAGATGAGCATTTTGCAGCTTTGTCAACTGAAAT
AGCTGCAGTAGAAAGCCAAGGTCAAATAAATCCTGTCCAACCCAGTGAAATCAC
AAATATTATCTTTCTTACAGTTTCAAGCACACAGCTTCCTGGAGGAGCTGGTTACC
TGAACTTTGGCAACAAATTACAAATTACCAATTCAAAAGGAAGTCCTAATGTCCC
ATCTAGTTCTCTCAACCCACTTTTTCCATCTTTTTCCTTTATTGTTCCTTGTTTTTTC
CCATTTTCATGTTCTTTGTCCCTTACTTGTTCAGTCTTTTCTTCTTGCACATTTTCTT
CACCATGTACTTTTCCTTCTTGCTCAGTTCTTCCTATTGTGGGTCTCTCTCCTGTTC
CTCCCTTAGCTGCTTCTGATAGTTCTCTCCAGAAACCATCTTCCTCGAAAGTTATT
GAAGACACCATTCTTTCCCATACTTTTTCATCCTTTCATGCTGCTCCAGCCACTCTT
TTAACAAAGCAACCATCTCTGATGCCTGGATTTCAATTGGAAACCAAGTCTAATC
AGCCTGAACAAGATCTTCCTAAGTATTCTGAACTCAATATTTCCCTTGCTGAGTGT
CGCCTGGGTGTGGTCTGGAAAGAAAGTCTCCAGGCTTTCTCGCTCTTCAAGACAG
CTGTTATTTCTCATGAGATCACAGAGTGTGGATTACGCCCTGGCCTTGTTCCACAC
TGTCCCAACTGCTGGGAGGCTGAAGTGGGTGAATTCCCTTGGATGGTTTCTGTGC
AACTCTCTTTCTCCCATTTTTGTGCTGGTTCTATACTGAATGAACAATGGATTCTC
ACTACAGCTAGGTGTGCAAATTTCATAAAAAACTCAGAAGCACTGGCCCATGTCC
AGGTGGGGCTTATCGATCTTCAAGACCCTGCTCAAGCTCAAACTATAGGCATTCA
TCGTGCCATGCCCTACCTGGGCCCTAGAGGACCTCTGGGGCCTGGTCTAATCTTCT
TGAAGCAACCATTACATTTTCAACCCCTGGTTCTTCCTATCTGCCTGGAGGAGAG
CCTAGAGCAAGAGAAAAATATACAACTGTATGACTGCTGGCTACCCAGTTGGTCC
CTCATGAGAGGAAGTCCTGGAATTTTGCAAAAAAGGCACCTGAGCATCCTGCAA
GCCATCACATGTGCCCAGTTTTGGCCCAAACTGAATGAATTTACTTTCTGTGTGGC
AGCCAAGAAAGCTATGGGGGAGGCTGGCTGTAAGGGTGACCTGGGGGCACCTCT
TGTGTGTCATCTGCAACAAAAAGACACATGGGTGCAGGTGGGAATTTTGACTCAC
TTTGATGAACACTGCACAAAGCCCTACGTCTTCAGCCAAGTGAGCCCTTTCCTTTT
CTGGCTCCAGGGAGTTACACGACCTAGCCAAGCACCCTGGTCCAAGCAAGGGCC
CATGACCACCTCTGCTTCAGTCTCCCTTTCAGTCTCTACCTCTACGAATGCCTCAG
CTTTTACTTCCACACCTGCTTCTGTCCGGCCACATTTCATCTCTCTGCCACAGCCTC
AGACTTTGGCAGATCGAATTTCTCTGAGATATGCCATGCCTTGGCAGGCCATGAT
CATCAGTTGTGGCAGTCAAATTTGTAGTGGTTCCATTGTTAGCAGCTCTTGGGTAC
TCACTGCGGCCCACTGTGTCAGGAATATGAATCCTGAAGACACAGCTGTAATATT
GGGTCTGAGGCACCCTGGGGCACCTCTGAGAGTTGTTAAGATCTCTACCATTCTT
CTGCATGAGAGATTTCGATTGGTGAGTAGGGCAGCAAGAAACGATCTAGCATTG
CTGCTCCTTCAAGAGGTCCAGACTCCCATTCAGATTTTAGCACCGCTAGGTCATCT
GAAGAATCTGAACAGCTCAGAATGCTGGCTGTCTGGGCCACGAATTCTTAAGCCA
GGAGAGACAGATGAAAATCCAGAAATATTACAGATGAAGGTGATAGGAGCTTCA
AGCTGTGCCCACCTTTACCCTGATATAGGCAGTTCTATTGTGTGCTTCATTACACA
AGACAAAGACGCTGACACAAATGTGGAACCAGTGAGTCCAGGCAGTGCTGTCAT
GTGCAGACCAATGTCTAGGAATGGAAGCTGGAGACAGATAGGCCTCACTAGTCT
GAAGGCACTGGCTACCATTGTGAGCCCCCACTTCTCATGGATATTATCCACTTCAT
CAAAGGCAGGACATCCATTAAACCATGCACTCATGCCTTGGATGGAAAAGCCTA
AGTCCTCTAGTCTTGTAAAACAGCCAACCACCCTGCCATTTTGTTCAACAATAATT
GTTATACTACAAAGGCTTTCATAACTCATTGCAAAAATAAGGCAGGGCTAATCTA
TTCAAACTATTCATAATAAAAATGTTAAACAATGTTAAAAAAAATTAAGACCCTA
TGCAACCTAGGAATATATGTCATGAATAAAC

>Goat (assembled from testis RNA-seq: accession numbers SRX1947628. SRX 1947629. SRX 1947630) (SEQ ID NO: 14)

CACAGATTGAAAGTCCAGATATAAACACCTGGAGAGAGACTGTGC
CTTTCACAGCCTCACAATGGACAAAGGCTGAACCTCCAGCTATAAATTCCTGGAG
AGAGAATATGCCTTTTTCAGTCCCACCATGGACACAGGATGGAAGTCCAGGTGTA
ATTACCTGGGGAGAGACTGTCACTTTCAGAGACCCACAAAGGACACAAATTGAA
AGTCCAGATATAAACACCTGGAGAGAGACTGTGCCTTTCATAGCACTACCATGGA
CACAGGCTGAAGGTCCAGATGTAAATACCCAGAGAGATACTTTGCCTTTTACAGG
TCTACGTTGGACTCAGGCTGAAAGTACAGCTGTGCATACTGGGAGAGATATTGTG
CCTTTCACAGCCCCATCATGGACACAGGCTGAACCTCCAGCTGTAAATTCCTGGA
GAGAGATTATGCCTTTTTCAGTCCCATCATGGACACAGGATGGAAATCCAGGTGT
AATTACCTGGGGAGAAACTGTCCCTTTCAGAATCCCACCAAAGTCACAGATTGAA
AGTCCAGATGTAAACACCTGGAGAGAGACTATGCCTTTCATAGCACTACCATGGA
CACAGGCTGAAGGTCCAGATGTAAATACCCAGAGAGATACTGTGCCTTTTGCAGG
TCTACACTGGACTCAGGCTGAAAGTACAGCTGTGCATACTGGGAGAGATATTGTG
CCTTTCACAGCCCCATCATGGACTCAAGATAAAGGCCCAGATGTAAATAACTGGA
GTGAGATGCTGAGTTTCACAGGACCATCATGGGTACATGCTAAAGGTCCAGCTTT
AAATACCTGGACAGAGACTGTCTGGAGGGAGACTGTACCTTTAACAGTCTCACCT
TGGACTCAGACTGAAAATCCAACTGTAAATACCTGGAGAGAGAATATGCTTTTAA
CAGCCCTACCCTGGTCACAGGTTGAATATCCAGCTGTAAATACATGGAGAGAAAC
CGTACATTTCACAGAGCCACCATGGAATCAGGCTGAATATCCTGCTGTAAACACC
TGGAGAGAAACTGTGCCTTTCTCAGCTCTACAATGGACTCAGGCTGAAAGTCCAG
CTGTAAACACCTGGAGAGAGGCTATGCCTTTCACAGACCCATCATGGATTCAAGA
TAAAAGTCCAACTGTAAATAGCTGGAGACGGATTTTTACTTTCCCAGCCCAACCA
TGGCCACAGACTGAAAGTACAGTGGAAAACGACTTGATATGGAATGCCCCTTTTA
CAGCTCCACCGTGGTCACACACTGAAAGTCCAGCTGTAAATACCTGGACAGAGCC
TATGCTTTTTATCGCCCCTCCATGGACTCAGGCTGAAAATCCATCTACAAATACCT
GGAAAGTGAATATGCATTACAGAGACCCACTATGGCCTCAGTCTGACTTTGCACC
AGCAAACCCTTGGACATCAACTGAAAGTTTTAGAATCACATCATGGACTCTTACA
GGAAAGCAAGTTTTAAATATTTGGACAGAGCCAATAGCTTCCACAGCCACACTGT
GGACTCAGGCTGAATATTCAACACCAAAATATTGGACAGAGACTAAGGCCATTT
ATATAGTCACATCATTGACCCAGTGTCAGTTTCCAATAAATACTTTGACAGAATC
TGTAGGAGCCATAATCACACTTTGTACATCTGCTGAATCTCTATCATTAAGTTCTT
TCACACAGAATATTATTGATACAATTGAATTTTGGCCAATGGTTAAAACTGAGTC
TAAGAAAAGGTGGAATCTGCCTCAAACTAGTACATTCATGTTTTCACTAAATCCT
CAAACTGATACTTTTGGATCCTTGAACCAAATTGAAAATCAAGAATCTCCTCTGT
GGACCCATCCTGAAATTGATAATGTCAATACAATGGCGTTTCTTGAATTTGGAAC
ACTCATATCACAGGTAGTACCTTTGCCCCAAGCAGCTAGATTCTGGCCCCAAACT
GAAGCTGATACTAGCAAAATTTGGTTTGTCTCCTCTGAAAGAATAAATTCCTGGG
ACCAATCAGAGTCTCAAAGAATGAGTACCTCAATCCATTTTGGAGTGGGTAGAGT
GAAGCCCCTCGCCCAACATGAAACTGCTACAGTCATGTCATGGCTTCAGATTGAA
ACTGGTATATTCCACCCTTGGAACAAGTCTGAAGGAGGCACAGGGAGGTTCTGGC
CCCTTTCTGAAACTGAGGATGTAAGAGAATGGATCCAAACTGGAGCCGGTACAG
TTAACTCTTGGACTCAACTGAGAACTAATATAGTCAGAGCTTGGCCCCAAGCTGA
ATCTGAACTAGTCAGACCCTGGACACAAACTAAAACGAATGCAATCACACTATTG
ACCCAGACTGATGCTATCAAACCTTGGTTCCAAACTAAAATTAATGCACTAAGAG
AAGGGACCCAAACTCAATCTCAAATTGTTACTACTTGGATCCAAACACAGTTGCA
AATATTTCACCCCTGGATTCAGCCTAAAAGTGATTCAGTCAGATTTTGGACCCAG
CCTTGGATCCAAGCTGAAACCCACACAGTCAGACTCTATTATGAAATTACTATAA
GAAAATCATGGGCCTCATCTGAATCTCAGTCAGTCACATTTTCATCACTGAGTCA
AAATTCAGTTAAGAACTCATTTCACTTTGAATCTCAGATGACATGTTCCTGGGTCC
GAAATGAATTTGATATAATCAGTCCTTGGAATCAATATGAAACTAGTTCTGTTGG
ATCCTGGTTCCAGTCTGAAACTGGTACCTGTCAACCCTGGCTCCATATTGAATCTT
CTACAATCACACCATGGACCCAATATGAAACATTAGAGATCTCCCCTTCAACCAA
TCCTGAGACTGATACAGCAATAAGGAATTTGTTCCAGCCCCAAATTGATCTAATT
AGTACTTGGAATCAGCCTGAAGTAGACACAATCAGATTCTGGACCCAAGTTGAA
ACAGAAACAATTCCAATGTGGACCCAGATTGGAACTCAAGTAGTTAAACCTCTCA
ACTTTTCTGAAGTTGGTATAGTTACACCTTGGCTAAAGACTGAAACTGATGCAAG
TAGACCCTGGATTCAGTCTGACTTTCAGTCAATCCATCCGTGGAGCCAGATTGGA
TTTGGTATAATTGCCCCCTGGTCTCAGCCAGGAGCTTCTGTAAATCAACCCTGGA
CCTTTGTTCAAACACAGTCAATCAGACCTTGGATTACAGTGGAATCCAATAGAAT
CAAATATTGGTTTCATGTTCCAATGAAAAAAGTCAGACTGAGGATTCCTTCTGAG
TCTCAAATATTGAGTTTCTGGATGCAGTCTGATGTTAGTAGAGTTAATGCTTGGAT
CCAACCAGAAACCCAGGCAGTCAATCCTGGGGCTCATCCTAAAACTGGCAATGTT
GCATCCCTGACTATTCCTAACCCTGAAAGAGTCAGAATGTGGATCCAGCCTGAAA
CAGAAATAAGACTTGGCATCATTTATAAAACTAATATAGCCACATCATTTGCTTC
TGAAATTGAACCAGATGGAACAATTAGTTATTTTGATTCGTGGTCTATCCATGTA
ACATTTTTACCAATAGAAACTGTTACTTCCCTAGATGAGCATTTTGCAGCTTTGTC
AACTGAAATAGCTGCAGTAGAAAGCCAAGGTCAAATAAATTCTGTCCAACCCAG
TGAGATCACAAATATTCTCTTTCTTACAATTGCAAGCACACAGCTTCCTGGAGGA
TTTGGTTACCTGAACTTTGGCAACAAATTACAAATTACCAATTCAAAAGGAAGCC
CTAATGTCCCATATAGTTCTCTCAACCCACTTTTTCCGTCTTTTTCCTTTCCTGTTC
CTTGTTTTTTCCCATTTTCATGTTCTTTGTCCCTTACTTGTTCAGTCTTTTCTTCTTG
CACATTTTCTTCACCATGTACTTTTCCTTCTTGCTCAGTTCTTCCTATTGTGGGTTT
CTCTCCTGTTCCTCCCTTAGCTGCTTCTGATAGTTCTCTCCAGAAACCATCTTCCTC
AAAAGTTATTGAAGACACCATTCTTTCCCATACTTTTTCATCCTTTCATGCTGCTC
CAGCCACTCTTTTAACAAAGCAACCATCTCTGATGCCTGGATTTCAATTGGGAAC
CAAGTCTAATCAGCCTGAACAAGATCTTCCTAAGTATTCTGAACTCAATATTTCCC
TTGCTGAGTGTCGCCTGGGTGTGGTCTGGAAAGAGAGTCTCCAGGCTCTCTCGCT
CTTCAAGACAGCTGTTATTTCTCATGAAATCACAGAGTGTGGATTGCGCCCTGGC
CTTGTTCCACACTGTCCCAACTGCTGGGAGGCTGAAGTGGGTGAATTCCCTTGGA
TGGTTTCTGTGCAACTCTCTTTCTCCCATTTTTGTGCTGGTTCTATACTAAATGAAC
AATGGATTCTCACTACAGCTAGATGTGCAAATTTCATAAAAAACTCAGAAGCACT
GGCCCATGTCCAGGTGGGGCTTATAGATCTTCAAGACCCTGCTCAAGCTCAAACT
GTAGGCATTCATCGTGCCATGCCCTACCTGGGCCCTAGAGGACCTCTGGGACCTG
GTCTAATCTTCTTGAAGCAACCATTACATTTTCAACCCCTGGTTCTTCCTATCTGC
CTGGAGGAGAACCTAGAGCAAGAGAAAAATATACAACTGTATGACTGCTGGCTA
CCCAGTTGGTCCCTCATGAGAGGAAGTCCTGGAATTTTGCAAAAAAGGCACCTGA
GCATCCTGCAAGCCATCACATGTGCCCAGTTTTGGCCCAAACTGAATGAATTTAC
TTTCTGTGTGGCAGCCAAGAAAGCTATGGGGGAGGCTGGCTGTAAGGGTGACCT
GGGGGCACCTCTTGTGTGTCATCTGCAACAAAAAGACACATGGGTGCAGGTGGG
AATTTTGACTCACTTTGATGAACACTGCACAAAGCCCTACGTCTTCAGCCAAGTG
AGCCCTTTCCTTTTCTGGCTCCAGGGAGTTACACGACCTAGCCAAGCACCCTGGT
CCAAGCAAGGGCCCATGACCACCTCTGCTTCCATCTCCCTTTCAGTCTCTACCTCT
ATGAATGCCTCAGCTTTTACTTCCACGCCTGCTTCTGTCCGGCCACATTTCATCTC
TCTGCCACAGCCTCAGACTTTGGCAGATCGAATTTCTCTGAGATATGCCATGCCTT
GGCAGGCCATGATCATCAGTTGTGGCAGTCAAATTTGCAGTGGTTCCATTGTTAG
CAGCTCTTGGGTACTCACTGCAGCCCACTGTGTCAGGAATATGAATCCTGAAGAC
ACAGCTGTAATATTGGGCCTGAGGCACCCTGGGGCACCTCTGCGAGTTGTTAAGA
TCTCTACCATTCTTCTGCATGAAAGATTTCGGTTGGTGAGTAGGGCAGCAAGAAA
CGATCTAGCATTGCTGCTCCTTCAAGAGGTCCAGACTCCTATTCAGATTTTAGCAC
CGCTAGGTCATCTGAAGAACCTGAACAGCTCAGAATGCTGGCTGTCTGGGCCACG
AATTCTTAAGCCAGGAGAGACAGATGAAAATCCAGAAATATTACAGATGCAGGT
GATAGGAGCATCAAGCTGTGCCCACCTTTACCCTGATATAGGTAGTTCTATTGTG
TGCTTCATTACACAAGACAAAGATGCTGACACAAATGTGGAACCGGTGAGTCCA
GGCAGTGCTGTCATGTGCAGACCAATGTCTAGGAATGGAAGCTGGAGACAGATA
GGCCTCACTAGTCTGAAGGCACTGGCTACCATTGTGAGCCCCCACTTCTCATGGA
TATTATCCACTTCATCAAAGGCAGGACATCCATTAAGCCATGCACTCATGCCTTG
GATGGAAAAGCCTAAGTCCTCTAGTATCATGAAACAGCCAACCACTCTGCCATTT
TATTCAACAATAATTGTTATACTACAAAGGCTTTAATAACTCATTGCAAAAATAA
TGCAGGGCTAATCTATTCAAACTATTCATAATAAAAATGTTAACGTTAAAAAAAA
TAAGGCCCTAT

### REFERENCES

1. Lyttle T W. Segregation distorters. Annual review of genetics (1991) 25:511-57.
2. Jaenike J. Sex chromosome meiotic drive. Annu Rev Ecol Syst (2001) 32:25-49.
3. Fisher R A. The Genetical Theory of Natural Selection: Oxford University Press; 1930. 291 p.
4. Hall D W. Meiotic drive and sex chromosome cycling. Evolution (2004) 58:925-31.
5. Skaletsky H, Kuroda-Kawaguchi T, Minx P J, Cordum H S, Hillier L, Brown L G, Repping S, Pyntikova T, Ali J, Bieri T, Chinwalla A, Delehaunty A, Delehaunty K, Du H, Fewell G, Fulton L, Fulton R, Graves T, Hou S F, Latrielle P, Leonard S, Mardis E, Maupin R, McPherson J, Miner T, Nash W, Nguyen C, Ozersky P, Pepin K, Rock S, Rohlfing T, Scott K, Schultz B, Strong C, Tin-Wollam A, Yang S P, Waterston R H, Wilson R K, Rozen S, Page D C. The male-specific region of the human Y chromosome is a mosaic of discrete sequence classes. Nature (2003) 423:825-37.
6. Hughes J F, Skaletsky H, Brown L G, Pyntikova T, Graves T, Fulton R S, Dugan S, Ding Y, Buhay C J, Kremitzki C, Wang Q, Shen H, Holder M, Villasana D, Nazareth L V, Cree A, Courtney L, Veizer J, Kotkiewicz H, Cho T J, Koutseva N, Rozen S, Muzny D M, Warren W C, Gibbs R A, Wilson R K, Page D C. Strict evolutionary conservation followed rapid gene loss on human and rhesus Y chromosomes. Nature (2012) 483:82-6.
7. Hughes J F, Skaletsky H, Pyntikova T, Graves T A, van Daalen S K, Minx P J, Fulton R S, McGrath S D, Locke D P, Friedman C, Trask B J, Mardis E R, Warren W C, Repping S, Rozen S, Wilson R K, Page D C. Chimpanzee and human Y chromosomes are remarkably divergent in structure and gene content. Nature (2010) 463:536-9.
8. Soh Y Q, Alfoldi J, Pyntikova T, Brown L G, Graves T, Minx P J, Fulton R S, Kremitzki C, Koutseva N, Mueller J L, Rozen S, Hughes J F, Owens E, Womack J E, Murphy W J, Cao Q, de Jong P, Warren W C, Wilson R K, Skaletsky H, Page D C. Sequencing the mouse Y chromosome reveals convergent gene acquisition and amplification on both sex chromosomes. Cell (2014) 159:800-13.
9. Li G, Davis B W, Raudsepp T, Pearks Wilkerson A J, Mason V C, Ferguson-Smith M, O'Brien P C, Waters P D, Murphy W J. Comparative analysis of mammalian Y chromosomes illuminates ancestral structure and lineage-specific evolution. Genome Res (2013) 23:1486-95.
10. Hughes J F, Skaletsky H, Pyntikova T, Koutseva N, Raudsepp T, Brown L G, Bellott D W, Cho T J, Dugan-Rocha S, Khan Z, Kremitzki C, Fronick C, Graves-Lindsay T A, Fulton L, Warren W C, Wilson R K, Owens E, Womack J E, Murphy W J, Muzny D M, Worley K C, Chowdhary B P, Gibbs R A, Page D C. Sequence analysis in Bos taurus reveals pervasiveness of X-Y arms races in mammalian lineages. Genome Res (2020) 30:1716-26.
11. Skinner B M, Sargent C A, Churcher C, Hunt T, Herrero J, Loveland J E, Dunn M, Louzada S, Fu B, Chow W, Gilbert J, Austin-Guest S, Beal K, Carvalho-Silva D, Cheng W, Gordon D, Grafham D, Hardy M, Harley J, Hauser H, Howden P, Howe K, Lachani K, Ellis P J, Kelly D, Kerry G, Kerwin J, Ng B L, Threadgold G, Wileman T, Wood J M, Yang F, Harrow J, Affara N A, Tyler-Smith C. The pig X and Y Chromosomes: structure, sequence, and evolution. Genome Res (2016) 26:130-9.
12. Piovesan A, Antonaros F, Vitale L, Strippoli P, Pelleri M C, Caracausi M. Human protein-coding genes and gene feature statistics in 2019. BMC Res Notes (2019) 12:315.
13. Mueller J L, Skaletsky H, Brown L G, Zaghlul S, Rock S, Graves T, Auger K, Warren W C, Wilson R K, Page D C. Independent specialization of the human and mouse X chromosomes for the male germ line. Nat Genet (2013) 45:1083-7.
14. Soh Y Q, Junker J P, Gill M E, Mueller J L, van Oudenaarden A, Page D C. A Gene Regulatory Program for Meiotic Prophase in the Fetal Ovary. Plos Genet (2015) 11: e1005531.
15. Lesch B J, Silber S J, McCarrey J R, Page D C. Parallel evolution of male germline epigenetic poising and somatic development in animals. Nat Genet (2016) 48:888-94.
16. Green C D, Ma Q, Manske G L, Shami A N, Zheng X, Marini S, Moritz L, Sultan C, Gurczynski S J, Moore B B, Tallquist M D, Li J Z, Hammoud S S. A Comprehensive Roadmap of Murine Spermatogenesis Defined by Single-Cell RNA-Seq. Developmental cell (2018) 46:651-67 e10.
17. Cardoso-Moreira M, Halbert J, Valloton D, Velten B, Chen C, Shao Y, Liechti A, Ascencao K, Rummel C, Ovchinnikova S, Mazin P V, Xenarios I, Harshman K, Mort M, Cooper D N, Sandi C, Soares M J, Ferreira P G, Afonso S, Carneiro M, Turner J MA , VandeBerg J L, Fallahshahroudi A, Jensen P, Behr R, Lisgo S, Lindsay S, Khaitovich P, Huber W, Baker J, Anders S, Zhang Y E, Kaessmann H. Gene expression across mammalian organ development. Nature (2019) 571:505-9.
18. Conway S J, Mahadevaiah S K, Darling S M, Capel B, Rattigan A M, Burgoyne P S. Y353/B: a candidate multiple-copy spermiogenesis gene on the mouse Y chromosome. Mammalian Genome (1994) 5:203-10.
19. Cocquet J, Ellis P J, Mahadevaiah S K, Affara N A, Vaiman D, Burgoyne P S. A genetic basis for a postmeiotic X versus Y chromosome intragenomic conflict in the mouse. Plos Genet (2012) 8: e1002900.
20. Kruger A N, Brogley M A, Huizinga J L, Kidd J M, de Rooij D G, Hu Y C, Mueller J L. A Neofunctionalized X-Linked Ampliconic Gene Family Is Essential for Male Fertility and Equal Sex Ratio in Mice. Curr Biol (2019) 29:3699-706 e5.
21. Cocquet J, Ellis P J, Yamauchi Y, Riel J M, Karacs T P, Rattigan A, Ojarikre O A, Affara N A, Ward M A, Burgoyne P S. Deficiency in the multicopy Sycp3-like X-linked genes Slx and Slx11 causes major defects in spermatid differentiation. Mol Biol Cell (2010) 21:3497-505.
22. Helleu Q, Gerard P R, Montchamp-Moreau C. Sex chromosome drive. Cold Spring Harb Perspec Biol (2015) 7: a017616.
23. Shang X, Shen C, Liu J, Tang L, Zhang H, Wang Y, Wu W, Chi J, Zhuang H, Fei J, Wang Z. Serine protease PRSS55 is crucial for male mouse fertility via affecting sperm migration and sperm-egg binding. Cell Mol Life Sci (2018) 75:4371-84.
24. Zhu F, Li W, Zhou X, Chen X, Zheng M, Cui Y, Liu X, Guo X, Zhu H. PRSS55 plays an important role in the structural differentiation and energy metabolism of sperm and is required for male fertility in mice. J Cell Mol Med (2021) 25:2040-51.
25. Courret C, Chang C H, Wei K H, Montchamp-Moreau C, Larracuente A M. Meiotic drive mechanisms: lessons from Drosophila. Proc Biol Sci (2019) 286:20191430.
26. Kruger A N, Mueller J L. Mechanisms of meiotic drive in symmetric and asymmetric meiosis. Cell Mol Life Sci (2021) 78:3205-18.
27. Loytynoja A. Phylogeny-aware alignment with PRANK. Methods Mol Biol (2014) 1079:155-70.
28. Guindon S, Delsuc F, Dufayard J F, Gascuel O. Estimating maximum likelihood phylogenies with PhyML. Methods Mol Biol (2009) 537:113-37.
29. Edgar R C. MUSCLE: multiple sequence alignment with high accuracy and high throughput. Nucleic Acids Res (2004) 32:1792-7.
30. Patro R, Duggal G, Love M I, Irizarry R A, Kingsford C. Salmon provides fast and bias-aware quantification of transcript expression. Nat Methods (2017) 14:417-9.
31. Langmead B, Salzberg S L. Fast gapped-read alignment with Bowtie 2. Nat Methods (2012) 9:357-9.

1. A method of biasing the sex ratio of offspring of a male mammal, comprising i) contacting the mammal with an agent that modulates the expression or activity of a testis specific gene located in a male-specific region of the Y chromosome (MSY) having an x-linked homolog, or the x-linked homolog thereof, wherein the mammal is not a mouse and/or ii) contacting the mammal with an agent that modulates the expression or activity of PRSSLY or a PRSSLY homolog.

2. The method of claim 1, wherein the testis specific gene has at least two copies on the MSY or at least ten copies on the MSY.

3. (canceled)

4. The method of claim 1, wherein the x-linked homolog comprises at least two copies or at least ten copies.

5. (canceled)

6. The method of claim 1, wherein the testis specific gene is located in an ampliconic region.

7. The method of claim 1, wherein the mammal is bovine, porcine, gorilla, feline, equine, or human, or wherein the mammal is an ungulate.

8. The method of claim 1, wherein the sex ratio is biased to males or toward females.

9. (canceled)

10. The method of claim 1, wherein the agent comprises a targeting endonuclease or an RNAi agent or antisense oligonucleotide specifically reducing or eliminating the expression of the testis specific gene or x-linked homolog thereof or an RNAi agent or antisense oligonucleotide specifically reducing or eliminating the expression of a gene product of PRSSLY or a PRSSLY homolog.

11. (canceled)

12. The method of claim 1, wherein the agent comprises a nucleic acid, protein or small molecule that modulates the activity of a gene product of the testis specific gene or x-linked homolog thereof or a gene product of PRSSLY or a PRSSLY homolog.

13. (canceled)

14. The method of claim 1, wherein the testis specific gene is HSFY or HSFX.

15. (canceled)

16. The method of claim 1, wherein the agent is locally administered to the mammal's reproductive cells.

17. A non-human mammal comprising i) a non-naturally occurring mutation in a testis specific gene located in a male-specific region of the Y chromosome (MSY) having an x-linked homolog, or the x-linked homolog thereof, ii) a non-naturally occurring nucleotide sequence capable of expressing an RNAi agent that reduces expression of a testis specific gene located in a male-specific region of the Y chromosome (MSY) having an x-linked homolog, or the x-linked homolog thereof, iii) a non-naturally occurring mutation that modulates the expression or activity of PRSSLY or a PRSSLY homolog as compared to a control non-human mammal, or iv) a non-naturally occurring nucleotide sequence capable of expressing an RNAi agent that reduces expression of PRSSLY or a PRSSLY homolog as compared to a control non-human mammal.

18. The non-human mammal of claim 17, wherein the testis specific gene has at least two copies on the MSY or at least ten copies on the MSY.

19. The non-human mammal of claim 17, wherein the non-human mammal is transgenic.

20. The non-human mammal of claim 17, wherein the x-linked homolog comprises at least two copies or at least ten copies.

21. (canceled)

22. The non-human mammal of claim 17, wherein the testis specific gene is located in an ampliconic region.

23. (canceled)

24. The non-human mammal of claim 17, wherein the mutation reduces expression or activity of a gene product of the gene or x-linked homolog thereof.

25. The non-human mammal of claim 17, wherein the non-human mammal comprises no functional copies of the testis specific gene or the x-linked homolog thereof or comprises at least 10 less functional copies of the testis specific gene or the x-linked homolog thereof than a wild-type mammal.

26. (canceled)

27. (canceled)

28. The non-human mammal of claim 17, wherein the RNAi agent is an siRNA or a microRNA.

29. The non-human mammal of claim 17, wherein the RNAi agent is expressed under the control of a tissue specific promoter.

30. (canceled)

31. A method of screening for a candidate agent that biases the sex ratio of offspring of a mammal, comprising:
a. providing a composition comprising a cell or cell free expression system expressing the product of PRSSLY, a PRSSLY homolog, a testis specific gene located in a male-specific region of a Y chromosome (MSY) of a mammal having an x-linked homolog, or the x-linked homolog thereof;
b. contacting the composition with a test agent; and
c. measuring the expression or activity of the product,
wherein if the expression or activity of the product is modulated as compared to a control then the agent is identified as a candidate agent that biases the sex ratio of offspring of a mammal.

32.-54. (canceled)


SEQUENCE LISTING (14 sequences)

SEQ ID NO: 1 - DNA, 1192 bp, Feline
tgagacattctcatggcaaaagtgctcttggtctagactagctccatggctagtcagagtgctaaggagatacccaaagggaagctggccccatctgatgatggagagccagcaccagaattaccatctagttcatcccaggatccaaatttggattccggggagattttggtgatgaatagggaccaagcagtgatccaagatccaggcccccaagacaacccacaaccacaggccccaaaccaaggcgccgccaacgtgggagaaaacaacagtattcttgggctctccttcccaagaaagctttggatgatcctggaggacaacaccttcacgtccgtgcgctggaacgatgccggggacaccgtgatcatcgacgaagaccttttccagagggaggttcttcaccgcagaggcgcggagagaatctttgaaactgacagcttgaagactttcatccgcctgctgaacctgtacggcttcagcaaaatacgcgatttaccgcaacgccaactttcagagagacaagcctttcctgctcaggaacattcagaggaaaagtgacctgagagtcaccaccacctggctaggcaccagtgcaccaactccaaagagaaagaagccggtggcagcaacaagacagtccccacgaatccatcacgaggaacccgccaacgacgacaagacggtcctcggcgcagccccaaatgctcagggtcccagcagcagccagcccttcgccttctctggcatttggtctctgagcagcgcagccgggtatgccatggcaactcatggcccgagtgagccaggtggcctgagtggggagggcacctccaggaacatgatgtttgtgcccctggctactgccagaagggatgacacaggggaactgcccgtcagccccccagtttaccccgactatggtacagtgatgtctctgtataacacctgttattccatcctgctggcagccctgtcagtcatgtccccaaatgaggcccccagtgagaacgaggagcaggagggctcctcagattacaagtgtgccctctgtgaacatttcaaggaaaatccaggtccctaggctcacaggccactgatgacagctaaaaagtcaccactgtaaccatcaatctattttcggctttaatggaaacaaacacaactccgcgggagtaaataaacagtc

SEQ ID NO: 2 - DNA, 1286 bp, Bovine
gagtatttatggggcaccccgaccatggtctgaggcccgttttccctgagcactcactgcttggatagataatgctccttagctagactggctccatggctagtcagagttcccatgaggcacgggcagccctgctgatcctatcaactgatggggagcctgcagcaggggacacccgtgattcctccccagatccaaacctggatttaggagaggctttggagaagcagggtgaccagcccaagagcccagatccaggcctccatgacaatccactccaacagggcccaaacccagaaatggccaaagaggaagagaacaacgccatccttgggctgtccttccccaggaagctctggaggattgtggaggatgcagccttcacctctgtgcactggaacgatgagggagatacagtggtcatcgaggcagatctcttccagatagaggtactccagcgcagaggcatggaccagatcttcgagacagacagcatcaagagcttcatccgtgaactgaatctgtacgggttcaggaaagtccgcccttcgagtcactctacagggaagaagctcatgatctatcgaaactccaattttcagagagacaagcctctccttctgcataacatccagaggaaaggcaaccccagaacaacttctcagcctgccactggcacaacaactccaaagagaaagaagcgagtggtggcaaccagacactctcctcaattccaccacaacgagttcacccaaaaggctggcaacaaagtccagaaggggatgccaactgctcgcagaacccccagccagtgctcatttgtgttctctgacctgtctgtgggcagtgtagccaggcgggctggggaaaaccatctccccagtgagcagggcagccccagcagggcggctggagagggcacatccagcaatgccatatctgtgccctcggctactgctgaaagggacagcccagggaaactgcccgagagccccccagtgtacccagattacgaatcggtgatggctttgtacaacacctgttactccatcctgatggcgggccttttagtcatggcaccagatgaggcccctgaggcggaggatgagcagggagagtcctcacattataagtgtgccctctgtgagcagctcaagaacaagcccaatccctgagctgccagatacctagggacactcaaaagcactctcttataattaaaaatagatttctggctctaataaagtcaagcaaaactccattggagtaaataaacaatcaaacga

SEQ ID NO: 3 - DNA, 1286 bp, Bovine
gagtatttatggggcaccccgaccatggtctgaggcccgttttccctgagcactcactgcttggatagataatgctccttagctagactggctccatggctagtcagagttcccatgaggcacgggcagccctgctgatcctatcaactgatggggagcctgcagcaggggacacccgtgattcctccccagatccaaacctggatttaggagaggctttggagaagcagggtgaccagcccaagagcccagatccaggcctccatgacaatccactccaacagggcccaaacccagaaatggccaaagaggaagagaacaacgccatccttgggctgtccttccccaggaagctctggaggattgtggaggatgcagccttcacctctgtgcactggaacgatgagggagatacagtggtcatcgaggcagatctcttccagatagaggtactccagcgcagaggcatggaccagatcttcgagatagacagcatcaagagcttcatccgtgaactgaatctgtacgggttcaggaaagtccgcccttcgagtcactctacagggaagaagctcatgatctatcgaaactccaattttcagagagacaagcctctccttctgcataacatccagaggaaaggcaaccccagaacaacttctcagcctgccactggcacaacaactccaaagagaaagaagcgagtggtggcaaccagacactctcctcaattccaccacaacgagttcacccaaaaggctggcaacaaagtccagaaggggatgccaactgctcgcagaacccccagccagtgctcatttgtgttctctgacctgtctgtgggcagtgtagccaggcgggctggggaaaaccatctccccagtgagcagggcagccccagcagggcggctggagagggcacatccagcaatgccatatctgtgccctcggctactgctgaaagggacagcccagggaaactgcccgagagccccccggtgtacccagattacgaatcggtgatggctttgtacaacacctgttactccatcctgatggcgggccttttagtcatggcaccagatgaggcccctgaggcggaggatgagcagggagagtcctcacattataagtgtgccctctgtgagcagctcaagaacaagcccaatccctgagctgccagatacctagggacactcaaaagcactctcttataattaaaaatagatttctggctctaataaagtcaagcaaaactccattggagtaaataaacaatcaaacga

SEQ ID NO: 4 - DNA, 1286 bp, Bovine
gagtatttatggggcaccccgaccatggtctgaggcccgttttccctgagcactcactgcttggatagataatgctccttagctagactggctccatggctagtcagagttcccatgaggcacgggcagccctgctgatcctatcaactgatggggagcctgcagcaggggacacccgtgattcctccccagatccaaacctggatttaggagaggctttggagaagcagggtgaccagcccaagagcccagatccaggcctccatgacaatccactccaacagggcccaaacccagaaatggccaaagaggaagagaacaacgccatccttgggctgtccttccccaggaagctctggaggattgtggaggatgcagccttcacctctgtgcactggaacgatgagggagatacagtggtcatcgaggcagatctcttccagatagaggtactccagcgcagaggcatggaccagatcttcgagacagacagcatcaagagcttcatccgtgaactgaatctgtacgggttcaggaaagtctgcccttcgagtcactctacagggaagaagctcatgatctatcgaaactccaattttcagagagacaagcctctccttctgcataacatccagaggaaaggcaaccccagaacaacttctcagcctgccactggcacaacaactccaaagagaaagaagcgagtggtggcaaccagacactctcctcaattccaccacaacgagttcacccaaaaggctggcaacaaagtccagaaggggatgccaactgctcgcagaacccccagccagtgctcatttgtgttctctgacctgtctgtgggcagtgtagccaggcgggctggggaaaaccatctccccagtgagcagggcagccccagcagggcggctggagagggcacatccagcaatgccatagctgtgccctcggctactgctgaaagggacagcccagggaaactgcccgagagccccccggtgtacccagattacgaatcggtgatggctttgtacaacacctgttactccatcctgatggcgggccttttagtcatggcaccagatgaggcccctgaggcggaggatgagcagggagagtcctcacattataagtgtgccctctgtgagcagctcaagaacaagcccaatccctgagctgccagatacctagggacactcaaaagcactctcttataattaaaaatagatttctggctctaataaagtcaagcaaaactccattggagtaaataaacaatcaaacga

SEQ ID NO: 5 - DNA, 1286 bp, Bovine
gagtatttatggggcaccccgaccatggtctgaggcccgttttccctgagcactcactgcttggatagataatgctccttagctagactggctccatggctagtcagagttcccatgaggcacgggcagccctgctgatcctatcaactgatggggagcctgcagcaggggacacccgtgattcctccccagatccaaacctggatttaggagaggctttggagaagcagggtgaccagcccaagagcccagatccaggcctccatgacaatccactccaacagggcccaaacccagaaatggccaaagaggaagagaacaacgccatccttgggctgtccttccccaggaagctctggaggattgtggaggatgcagccttcacctctgtgcactggaacgatgagggagatacagtggtcatcgaggcagatctcttccagatagaggtactccagcgcagaggcatggaccagatcttcgagacagacagcatcaagagcttcatccgtgaactgaatctgtacgggttcaggaaagtccgcccttcgagtcactctacagggaagaagctcatgatctatcgaaactccaattttcagagagacaagcctctccttctgcataacatccagaggaaaggcaaccccagaacaacttctcagcctgccactggcacaacaactccaaagagaaagaagcgagtggtggcaaccagacactctcctcaattccaccacaacgagttcacccaaaaggctggcaacaaagtccagaaggggatgccaactgctcgcagaacccccagccagtgctcatttgtgttctctgacctgtctgtgggcagtgtagccaggcgggctggggaaaaccatctccccagtgagcagggcagccccagcagggcggctggagagggcacatccagcaatgccatatctgtgccctcggctactgctgaaagggacagcccagggaaactgcccgagagccccccggtgtacccagattacgaatcggtgatggctttgtacaacacctgttactccatcctgatggcgggccttttagtcatggcaccagatgaggcccctgaggcggaggatgagcagggagagtcctcacattataagtgtgccctctgtgagcagctcaagaacaagcccaatccctgagctgccagatacctagggacactcaaaagcactctcttataattaaaaatagatttctggctctaataaagtcaagcaaaactccattggagtaaataaacaatcaaacga

SEQ ID NO: 6 - DNA, 1287 bp, Bovine
gagtatttatggggcaccccgaccatggtctgaggcccgttttccctgagcactcactgcttggatagataatgctccttagctagactggctccatggctagtcagagttcccatgaggcacgggcagccctgctgatcctatcaactgatggggagcctgcagcaggggacacccgtgattcctccccagatccaaacctggatttaggagaggctttggagaagcagggtgaccagcccaagagcccagatccaggcctccatgacaatccactccaacagggcccaaacccagaaatggccaaagaggaagagaacaacgccatccttgggctgtccttccccaggaagctctggaggattgtggaggatgcagccttcacctctgtgcactggaacgatgagggagatacagtggtcatcgaggcagatctcttccagatagaggtactccagcgcagaggcatggaccagatcttcgagacagacagcatcaagagcttcatccgtgaactgaatctgtacgggttcaggaaagtccgcccttcgagtcactctacagggaagaagctcatgatctatcgaaactccaattttcagagagacaagcctctccttctgcataacatccagaggaaaggcaaccccagaacaacttctcagcctgccactggcacaacaactccaaagagaaagaagcgagtggtggcaaccagacactctcctcaattccaccacaacgagttcacccaaaaggctggcaacaaagtccagaaggggatgccaactgctcgcagaacccccagccagtgctcatttgtgttctctgacctgtctgtgggcagtgtagccaggcgggctggggaaaaccatctccccagtgagcagggcagccccagcagggcggctggagagggcacatccagcaatgccatatctgtgccctcggctactgctgaaagggacagcccagggaaactgcccgagagccccccggtgtacccagattacgaatcggtgatggctttgtacaacacctgttactccatcctgatggcgggccttttagtcatggcaccagatgaggcccctgaggcggaggatgagcagggagagtcctcacattataagtgtgccctctgtgagcagctcaagaacaagcccaatccctgagctgccagatacctagggacactcaaaagcactctcttataattaaaaatagatttctggctctaataaagtcaagcaaaactccattggagtaaataaacaatcaaacgag

SEQ ID NO: 7 - DNA, 1286 bp, Bovine
gagtatttatggggcaccccgaccatggtctgaggcccgttttccctgagcactcactgcttggatagataatgctccttagctagactggctccatggctagtcagagttcccatgaggcacgggcagccctgctgatcctatcaactgatggggagcctgcagcaggggacacccgtgattcctccccagatccaaacctggatttaggggaggctttggagaagcagggtgaccagcccaagagcccagatccaggcctctatgacaatccactccaacagggcccaaacccagaaatggccaaagaggaagagaataacgccatccttgggctgtccttccccaggaagctctggaggattgtggaggatgcagccttcacctctgtgcactggaacgatgagggagatacagtggtcatcgaggcagatctcttccagatagaggtactccagcgcagaggcatggaccagatcttcgagacagacagcatcaagagcttcatccgtgaactgaacctgtacgggttcaggaaagtccgcccttcgagtcactctacagggaagaagctcatgatctatcgaaactccaattttcagagagacaagcctctccttctgcataacatccagaggaaaggcaaccccagaacaacttctcagcctgccactggcacaacaactccaaagagaaagaagcgagtggtggcaaccagacactctcctcaattccaccacaacgagttcacccaaaaggctggcaacaaagtccagaaggggatgccaactgctcgcagaacccccagccagtgctcatttgtgttctctgacctgtctgtgggcagtgtagccaggcgggctggggaaaaccatctccccagtgagcagggcagccccagcagggcggctggagagggcacatccagcaatgccatatctgtgccctcggctactgctgaaagggacagcccagggaaactgcccgagagccccccggtgtacccagattacgaatcggtgatggctttgtacaacacctgttactccatcctgatggcgggccttttagtcatggcaccagatgagacccctgaggcggaggatgagcagggagagtcctcagattataagtgtgccctctgtgagcagctcaagaacaagcccaatccctgagctgccagatacctagggacactcaaaagcactctcttataattaaaaatagatttctggctctaataaagtcaagcaaaactccattggagtaaataaacaatcaaacga

SEQ ID NO: 8 - DNA, 1286 bp, Bovine; identical to SEQ ID NO: 5

SEQ ID NO: 9 - DNA, 1286 bp, Bovine; identical to SEQ ID NO: 5

SEQ ID NO: 10 - DNA, 1286 bp, Bovine; identical to SEQ ID NO: 5

SEQ ID NO: 11 - DNA, 6043 bp, Bovine
agagactgcccctttcacagtcccaacatggactctggctaaaagaccagctgtaaaatcctggagagagaatgtgcctttcacaactccaccatggacattggctgaaggtccacctgtaaatacctggagagaaactctgcctttcataggcccaccttggactcatgctgaaagtacagctataaatagttggagagggactgtgccatttgcaactgcactatgggctctagataaaggtccagctgtaaatacctggagagagatgatgactttcacagccccaccatggacacaggctgaaggtccagctgtaaatacatggagagagactattccttttacagtcccaccatggactcaggatgaaagtccaggtgtaattacctggggagcaactgtccctttcagagtcccatcaaggacacagattgaaagtccagatataaacacctggagagagactgcacctttcacagccccagcatgggcacaggctgaacctccagctgtaaattcctggagagagattatgcctttttcagtcccaccatggacacaggatgaaaatccaggtgtaattacctggggagagactgtcactttcagagccccaccaaggacacagattgaaagtccagatgtaaacacctggagagagactgtgcctttcacagcactaccatggacacaggctgaaggtccagatgtaaatacccaaagagatactgtgcctttcacaggtctaccttggactcaggctgaaagtacagctgtgcatactgggagagatattgtacctttcacagctccaccatggactcaagataaaggcccagctttaaatacccggacagagactgtacctttaacaggctcaccttggactcaggctgaaaatccaactgtaaatacctggagagataagatgcctttaacagccccaccctggtcacaggttgaacgtccagctgtaaatacatggagagagactgtgcctttcacagcaccaccatggactcaggctgaacgtcctgctataaatagctggagagagactatgcctttctcagctctaccatggactcaggctgaaagtccagctgtaaacacctggagagacactatgcctttcacagacccatcatggactcaagataaaagtccagctgtaaatagctggagacagattttgactttcccagcccaaccttggccacaggctgaaagtatagctgcaaatgactggacacggaacgccccttttacagctccactgtggtcacacactgaaagtccagctgtaaatacctggacagaggctatgcttttcacaggccctccatggactcaggctgaaaatccagctacacatacctggaaagtgaacgtgcattacagaggcccaccatggactcagtctgactctgcacaagcaaacccttggacatcaactgaaagttttagaatcagatcatggactcatggagtaaagcaagttttgaatatttggacagagccaatagcttccacagttacacttcggactcaggctgaatattcaacactaaaatattgggcagagactaaagtcatttacatagtcacaccattgacccagtgtcagtttccaataaatactttgacagaatctgtaggatccataatcacaccttggacatctgctgaatctctagtattaagttctttcacacagaatattattgatataatcaaattttggccagtgcttaaaactgagtctaagaaaaggtggaatctgcctcaaactgatacactcatattttcactaaatcctcaaactgatacttttggatccttgaaccaaattgaaaatcaagaatctcctctgtggacacatcctgaaattgataatgtcaatacaatgaactttcttgaatctggaacactcatatcacaggtagtatctctgccccaagcagctagactctggccccaaactgaagctgatattagcaaaacttggtttgtatcctctgaaagaataaattcttgggaccaatcagagtctcaaagaatgagtacctcaacccattttggagtgggtagagtaaagcccttggcccaacatgaaactgctatagtcatgtcatggcttcagattgaaactggtatattctacccttggaaccagtctgagggagacacagtgaggttctggcccctttctgaaactgaggatgtaagagaatggatccaaactggagccagtagagttaactcttggactcaaccgagaactagtatagtcagagcttggccccaagctgaatctgaactagtcagaccctggacacaaactaaaactaatgcaatcacactattgacccaggctgatactatcaaaccttggttccaaactcaaattaatgcaataagagaaggagcccaaactcaatctcaaattgttactagtatccaaacacagttgcaaatagttaacccctggattcagcctaaaagtgattcaatcagattttggacccagccttggatccaagctgaaacccacacagtcagactcttttatgaaattgatataagaaaatcatgggcctcatttggatctcagtcagtcacattttggtcactgagtcaaaattcagttaggacctcatttcactttgaatctcagatgacatgttcctggatcggaaatgaatttgatataatcagtctttggaatcaatatgaaactagttcagttggatcctgcatcaagtctgaaactggtacatgtcaaccctgggtccatattgaatcttctacaatcacaccatggacccaatatgaaactttagagatctacccttcaacccagcctgagactgatacagcattaaggcattggttccagccccaaattgatccaattaatacttggaatcagcccgaagtagatacaatcagattctggacccaagttgaaacagaaacaatttcagtttggacccagattggaagtcaagtagttaaactttccaacttttctgaagttggcatagttacaccttggctagagactgaaactgatacaagtagaccctggattcagtctgactttcagtcagtccatccttggacccagactggatttggtataattaacccctggtatcagccaagagctgctgtaaatcaaccctggacatttgttcaaacacagtcaatcggaccctggactaaggtggaagccaatacaatcaaatcttggtttcatgttcaaatgaaaaaagtcagactggggattcctgctgagtctcaaatattgagtttctggatgcagtctgatgttagtagagttaatgcttggatccaaccagaaacccaggcagtcaatcctggggctcatcctaaatctggtaatgttgcatccctggctattcctaagcctgaaagagtcagaatgtggatccagcctgaaacagaaataaggcctggcatcatttataaaactgatataaccacatcatttgcttctcctgaaattgaaccagatggaacaattagtcattttgatttcttgtctaatcgtgtaacatttttaacaatagaaactgttccttccctagatgagcattttgcagctttgtcaactgaaatagctgcagtagaaagccaaggtcaaataaattctgtccaacccagtgagatcacaaatactctctttcttacactttcaagcacatggcttcctggaggagctggttacctgaactttgccaataaattgcaaattaccaaaacaaaaggaagccctaatgtcccatctagttctctcaacccactttttccatctttttcctttcctgttccttgttttatcccattttcatgttctttgtcccttacttgttcagtcttttcttcttgcacactttcttcaccatgtacttttccttcttgctcagttcttcctcttgtggctttctctcctgttcttcccttagctgcttctgatagttctctccagaaaccatcttcctcagaagttactgaagacaccattctttcccatactttttcatccttgcatgctgctccagccactcttttaacaaagcaaccatctctgatgcctggatttcaatctgaaaccaagtctaatcggcctgaacaagatcttcctaagtattctgaactcaatgtttcccttgctgagtgtcgcctggatgtggtctggaaagagagtctccaggctttctcgctcttcaagacagctgttatttctcatgaaatcacagagtgtggattacgccctggccttgttccacactgccccaactgctgggaggctgaagtgggtgaattcccttggatggtttctgtgcaactctctttctcccatttctgtgctggttctatactgaatgaacagtggattctcactacagctagatgtgcaaatttcataaaaaactcagaagcattggcccatgtccaggtggggctgattgatcttcaagaccctgctcaagctcaaactgtaggcattcatcgtgccatgccctacctgggccctagaggacctttgggacctggtctaatcttcttgaagcaaccattacattttcaacccctggttcttcctatctgcctggaggagaacctagagcaagagaaaaatatacaactatatgactgctggctacctagttggtccctcatgagaggaagtcctggaattttgcaaaaaaggcatctgagcatcctgcaagtcatcacatgtgcccagttttggcccagcctgaatgaatttactttctgtgtggcagccaagaaagctatgggggaggctggctgtaagggtgacctgggggcacctcttatatgtcatctgcaacaaaaagacacatgggtgcaggtgggaatcttgactcactttgatgaacactgcagaaagccctatgtcttcagccaagtgagccctttccttttctggctccagggagttacacgacccagccaagcaccctggtccaagcaagggcccatgaccacctctgcttccatctccctttcagtctccacctctatgaatgcctcagcttttacctccacacctgcttctgtccggccacatttcatctctctgccacagcctcagactttggcagatcgaatttctctgagatatgccatgccttggcaggccatgatcatcagttgtggcagtcaaatttgcagtggttccattgttagcagctcttgggtactcaccgcggctcactgtgtcaggaatatgaatcctgaagacacagctgtaatattgggcctgaggcaccctggggcacctctgagagttgttaagatctctaccattcttctgcatgaaagatttcgattggtgagtagggcagcaagaaacgatctagcattgctgctccttcaagaggtccagactcccattcagcttttagcaccgttgggtcatctgaagaacctgaacagctcagaatgctggctgtctgggccaagaattcttaagccaggagagacagatgaaaatccagaaatattacagatgcaggtgataggagcttcaagctgtgcccacctataccctgatataggtagttccattgtctgcttcattacacaagacaaagattctgacacaagtgtggaaccagtgagtccaggcagtgctgtcatgtgcagaccaatctctaggaatggaagctggagacagataggcctcactagtctgaaggcactggctaccattgtgagcccccacttctcatggatattatccacttcatcaaaagcagggcatccattaagccatgaactaatgccttggatggaaaagcctaagtcctctagtctcataaaacagccagccaccctgccattttattcaataataattgttatactacaaaagctttcataactcactgtgaaaataaggcagggctaatctattcaaactatttataataaaaattttaaacaacattaaagaaaattaagaccctatgcaacctaggag

SEQ ID NO: 12 - DNA, 5235 bp, Pig
ctcagagaaaaatcttggctgcagtctgattttgaaaaattctcaccttcaacccaggttcaatttgtattggtaaaaccctggacacagtctgaaaatgatacactcatgccatggacccaggttcaaccttctgcagtaaatgtctggacaaaggctacagctttcacagtcataccatggactcaagatgaattttttgcagaaaagtcctggacagagactgtggtttccatagtcacactgtggattaaggctacatctccagaagtaaatacctggacagaggtttctctttccacagtcacaccagggtctcaaactggatctccagaggtaaaaccctgggcagaggctctaacttccacagttatacccacactactgcctcaggatgaaatttctgcaacaaaatcctggacaaagaatgtgggtgccagagtcacactatggactcagcctgaatctccatctgtccatccctgggcacagactgtagcttccacagtcacactcacaccatggactcaggatgaattttctgtaacaaaattctggacagagacttttttttccacagttccacagtggactcaggctcaatctcccttaaatcctttgacagagactatagatgccatagtcacaccatggattcaggctgaatctccaggagtaaatcatttgacagaggctgtttcttccatagtcacacctacaccatggactcaggctaaagattcagcaataaaaccctggacacagactgtcacaccatggattcacactcaatctccagaagtaaatccctggacacagactgcagcttccacagctgcattgtggactcatattgaatctctagcattaagttattccgcacagagtattactgatacagttatattttggaccatgcttcaaactgaatctaagaaaccctggagtctttccaaacctagtttattcactatttcactgaatactcaaagtgatcttactggatcactgattcataatgaaaatcaagcatatcttctgttgacacattctgaaatttataatgtcaagacactgaacttgcctaaatctgaaacactcatatcatggacagtgccctttcctcaagcagtcagtctctggccccaatctgaagttgatattaccagaacttggtttaaaataccagaaagaaaaacatcctgggcccagtcagaatctctgacaatgagtaccatgacacagataggagatgctggaatgaggatgatagcacagcatgaacctgctgaagtcacatcatggattcagactgaaagtggtatattccacccttgggagaagtctgaaggagatatagtgagattctggaccccttctgaaactaagcctgtaaaaccatggatgcaaactggaactgctttagtcactccactgacccagcctgaaatgcaagcattaaaatacatgagcatgcctgacattaattctatcagaccttggttcaagactcaaactgagcctataagagaaggcactcaacctgaatctcaaatagtttctactaggatccaaccagaaccacaaatcatccatccatggttccagcctgaaagtaatgcagtcagattctggactcagcctaaaggtaatttagcccaatcctggtttcaagctaaagccaatatagtaagattccagactcagtatgaaattgatacaggaaaaccatggacccagcctgaatctcagtcagtcacatttttgtccttaacgcaaaatgatgcaattagtcccacatcccactttgaatctcagaagacgtgttcctggacccaaaatgaatttggtataatcagtctttggactcagtctgaaactagttcagtcatatttggaaccaagcttgaaactattacaggccaaccctggcttcatcttgaatctgctacatgcagaccatggaccctgcatgaaaccttaacccagcctgaagctgctacagtaagacactggctccatacccaaatggacttaaaagaaccttggaatcagcctgaagatgatgcagttatacactggacccagcttgaaacagaaacaatttatgtttggacccagactgaaagagaagtagtaaaacctccaactttatctgaagtcaatttagtcacatcttggttacagactcaaagtgatataattagaccttggattaaacctgagtttcagtcagtcattccttcgatccacactggagttgatatacttcacttctggtctcagcaaagagctgctacaaatcaacccagaacataccccaaaacccaggtagtcagaccatggatcaagctggaggctgatacaatcaaatcttggttccacattcaaaggaataaagtcagaccatggatgcctacaaaatctcaaatattgagcctctggaggcagcatgaagttgatatagtccatccatggatccagccagaaaccctggcagacagaccctggtcccactctgaaactgagattgcattcttggaaagttataaacctgaccaagttagaacatggatccagcctgaaatagaaataaggcctggcatccattataaagctgataaaatgacatcatttacttctcctgaagtagagcaaaatgaaacaacccaattaaccagtcactttggctcttggtataaacataaaccttttataccaacagaaagtattccttccccggatgagtattttacagctttgtcaactgagataactacacaagaaatccaagatcaaatcaattctgtccaacccactgagctcacagatattctctttttcaccctttcaagcatgtggtttcctgaaggagttggttatctaaaatttggcagtaaattacaaattaccaaaacaaaaggaagccctgagttcccatctacttctcagagccccctttctccatccctttcctttcctgttccatgtttttcaccatccctgtgttccttgtccctttcctgttcggtgttttcttcttgcgcattccctagaccctgtattttcccttcttgctcaagtctttcctctgtggtattctctcctattctccttcccttagctgcttctgatagttttctccagaaactatattactcaaaagttactgaagaaaccattctttctcatacttctccatccctgaatattgcttcagccatacttttaacaaagcagccttccctgatgtctggatctcaatctggaaccaagtctaatcaacctgaacaagatcctctcaagtattctgaactcaatatttccttggctgaatgtcacctgggtgtggtctggaaagagaatctccaagctttctggctattcaaaacagctgttatttcccatgaaaccacagagtgtggattacgccctggcctcgtcccacattgtcccaactgctgggaagcagaagtgggtgaattcccttggatggtttctgtgcaactgtctttctctcatttctgtgctggctccatactgaatgaacagtggatccttactaccgctagatgcgcaaatttcataaaaaactcagaagcactggcccttgtccaggtggggcttattgatcttcaagacactgcccaagctcaaacagtaggcattcatcgtgccatgccctacctaggtcctaagggccctttgggacctgggctgatcttccttaagcagccattacattttcaacctctggtgcttcctatctgcctggaagagagtctggagcaagagaaaaacatacaactgtatgactgctggctacctagttggtctctcatgagaggaagtcctggaattctgcaaaaaagacacctaagcatcctgcaagtcagcacgtgtgctcaattttggcccaatttgagtgaatttactttctgtgtagaagccaagaaagctatgggggaagctggctgtaagggtgacctgggggcacctttggtgtgccatctacaacaaaaggacacatgggtgcaggtgggaatattgagtcattttgatgaacattgcacaaagccctacgtcttcagccaagtgagccctttccttttctggctccagggagttacacgaccaagccatgctccctggtccaagcaagggcccatgactacttctgcttccatctcactttcagtatctacctctacgaatgcttcagcttttacctccactcctgcttctgtccagcctcacttcatctcactgccacagcctcagactctggcagatcgggtttctctaagatatgccatgccttggcaggccatgattatcagctgtggcagtcaaatttgcagtggttccattgttagcagctcctgggtactcactgctgctcactgtgtcaggaacatgaatcctgaagacactgctgtaatactgggcctgagacacccaggggcacctctgagagttgtcaaggtatccaccattttactgcatgagagattccggttggtaagtggagcagcaagaaatgacctagcattgctgctgcttcaagaggtacagactcccattcagcttttagcacccttaggccatctaaagaacttgaatagctcagaatgctggctctcaggaccacggattctaaagccaggagagacagatgaaaatccagaaatactgcagatgcaggtgataggagcttcaagctgtgcccacctctatccagacataggcagttctattgtgtgcttcattacacaggacaaagactccaatacaaatgtggaaccggtgagcccaggcagtgctgttatgtgccgaccaatgtctgggaatggaagctggagacagatagggctcaccagtctgaaagctctagctaccattgtgagaccccacttctcctggatattatccacttctgcaaaagcagggcatcccctaaaccaagcactcatgccttgggtagaaaaggccaaatcctctagtctcttaaaaaagccaaccatattaccatcagtaataattattgcaatacaaagccttttgtaagttaatagctatagagggaggagataatctgttcacactgtactggaaagtatattaacaatgtcataacaagactgatacatttagatagaactcagtaattctgaaataaagaaatactctatctaaatgactaatttcttttaaaacacttaaaaaacaaattat

SEQ ID NO: 13 - DNA, 5759 bp, Sheep
ctcaggatgacagtccaggtgtaattacctggggagagactgttcctttcagaaccacatcaaggacaccgattgaaagtccagatataaacacctggagagagactgtgcctttcacagcctcacaatggactcaggctgaacctccagctataaattcttggagagagatgatacctttttccatcccaccatggacacaggatggaagtccaggtgtaattacctggggagagactgtccctttcagagccccaccaaggacgcaaattgaaagtccagatgtaaacacctggagagagattgtgcctttcacagcactaccatggacacaggctgaaggtccagatgtaaatacccagagagatactgtgccttttacaggtcggactcaggctgaaagtacagctgtgcatactgggagagatactgtgcctttcacagctccaccatggactcaagataaaggcccagatgtaaataacagcagtgaggtgctgagtttcacaggaccatcattggcacatgctaaaggtccagctttaaataactggagggagactgtacctataataggctcatcctggactcaggctgaaaatccaactgtaaatgcctggagagagaatatgcctttaacagccccaccctggtcacaggttgaacatccagctataaatacatggagagaaactgtgcctttcacagagccaccatggactcaggctgaatatcctgctgtaaacacctggagagaaactgtgcctttctcagctctaccatggactcaggctgaaagtccagctgtaaacacctggagagaggctatgcctttcacagacccatcatggattcaagagaaaagtctaactgtaaatagctggagacagatttttactttcccagcccaaccatggccacagactgaaagtacagtggaaaacgactggatatggaatgccccttttacagctccaccatggtcacacactgaaagttcagctgtaaatacctggactgagcctatgctttttatagcccctccatggactgaggctgaaaatccagctacaaatacctggaaagtgaatatgcattacagagacacactatggcctcagtctgactttgcaccagcaaacccttggacatcaactgaaagtttcagaatcacctcatggactcatactgtaaagcaagttttaaatatttggacagagccaatagcttccacagccacactgtggactcaggctgaatattcaacaccaacatattggacagagattaaggccatttatatagtcacaccattgacccagtatcagttttcaataaatactttgacaaaatctgtaggagccataatcacactttggacgtctgctgaatctctgtcattaagttctttcacacagaatagtatcgatacaatcgaattttggccaatgcttaaaactgagtctaagaaaaggtggaatctgcctcaaactagtacattcatgttttcactaaatcctcaaattgatacttttggatccttgaaccaaattgaaaatcaagaatctcctcggtgggcccatcctgagattgataatgccaatacaatggcctttcttgaatttggaacactcatatcacaggtagtacctttgccccaagcagctagactctggccccaaactgaagctgctaatagcaaaatttggtttgtatcctctgaaagaataaattcctgggaccaatcagagtctcaaagaatgagtacctcaatccattttggagtgggtagagtgaagcccctggcccaacatgaaactgctatagtcatgtcatggcttcagattgaaactggtatattccacccttggaacaagtctgaaggaggcacagggaggttctggcccctttctgaaactgaggatataagagaatggatccaaactggagccagtacagttaactcttggactcaactgagaactaatatagtcagagcttgtccccaagctgaatctgaattagtcagaccctggacacaagctaaaactaatgcaatcacactattgacccagactgatgctatcaaaccttggttccaaactaaaattaattcactaagagaagggacccaaactcaatctcaaattgttactacttggatccaaacacagttgcaaatatttcacccctggattcagcctaaaagtgattcagtcagattttggacccagccttggatccaagctgaaacccacacagtcagactctattatgaaattgatgtaagaaaatcatgggcttcatctgaatctcagtcagtcacattttggtcactgagtcaaaattcagttaggacctcatttcactttgaatctcagatgacatgttcctgggtccgaaatgaatttgatataatcagtccttggaatcaatatgaaactagttctgttggatcctggatccagtctgaaactggtacatgtcaaccctggttccatattgaatcttctacaatcacaccatggacccaatatgaaactttagagatctccccttcaacccatcctgagactgatacagcaataaggcatttgttccagccccaaattgatccaattagtacttggaatcagcctgaggtagatacaatcagattctggacccaagttgaaacagaaacaattccaatttggatccagattggaagtcaagtagttaaacctcccaacttttctgaagttggtatagttacaccttggctaaagactgaaactgatgcaattattccctggattcagtctgactttcagtcaatccatccttcgacccagactggatttggtataattgacccctggtttcagccaagagcttctgtaaatcaaccctggacctttgttcaaacacagtcaatcagaccttggattaaagtggaatccaatacaatcaaatcttggtttcatgttccaatgaaaaaagtcagactggggattccttctgagtctcaaatattgagtttctggttgcagtctgatgttagtagagttaatgcttggatccaaccagaaacccaggcagtcaatcctggggctcatcctaaaactggtaatgttgcatccctgactattcctaagcctgaaagagtcagaatgtggatccagcctgaaagagaaatgagacctggcatcatttataaaactaatataaccacatcatttgcttctgaaattgaaccagatggaacaattagtcattttgattcctgggctaaccatgtaacatttttaccaatagaaactgttccttccctagatgagcattttgcagctttgtcaactgaaatagctgcagtagaaagccaaggtcaaataaatcctgtccaacccagtgaaatcacaaatattatctttcttacagtttcaagcacacagcttcctggaggagctggttacctgaactttggcaacaaattacaaattaccaattcaaaaggaagtcctaatgtcccatctagttctctcaacccactttttccatctttttcctttattgttccttgttttttcccattttcatgttctttgtcccttacttgttcagtcttttcttcttgcacattttcttcaccatgtacttttccttcttgctcagttcttcctattgtgggtctctctcctgttcctcccttagctgcttctgatagttctctccagaaaccatcttcctcgaaagttattgaagacaccattctttcccatactttttcatcctttcatgctgctccagccactcttttaacaaagcaaccatctctgatgcctggatttcaattggaaaccaagtctaatcagcctgaacaagatcttcctaagtattctgaactcaatatttcccttgctgagtgtcgcctgggtgtggtctggaaagaaagtctccaggctttctcgctcttcaagacagctgttatttctcatgagatcacagagtgtggattacgccctggccttgttccacactgtcccaactgctgggaggctgaagtgggtgaattcccttggatggtttctgtgcaactctctttctcccatttttgtgctggttctatactgaatgaacaatggattctcactacagctaggtgtgcaaatttcataaaaaactcagaagcactggcccatgtccaggtggggcttatcgatcttcaagaccctgctcaagctcaaactataggcattcatcgtgccatgccctacctgggccctagaggacctctggggcctggtctaatcttcttgaagcaaccattacattttcaacccctggttcttcctatctgcctggaggagagcctagagcaagagaaaaatatacaactgtatgactgctggctacccagttggtccctcatgagaggaagtcctggaattttgcaaaaaaggcacctgagcatcctgcaagccatcacatgtgcccagttttggcccaaactgaatgaatttactttctgtgtggcagccaagaaagctatgggggaggctggctgtaagggtgacctgggggcacctcttgtgtgtcatctgcaacaaaaagacacatgggtgcaggtgggaattttgactcactttgatgaacactgcacaaagccctacgtcttcagccaagtgagccctttccttttctggctccagggagttacacgacctagccaagcaccctggtccaagcaagggcccatgaccacctctgcttcagtctccctttcagtctctacctctacgaatgcctcagcttttacttccacacctgcttctgtccggccacatttcatctctctgccacagcctcagactttggcagatcgaatttctctgagatatgccatgccttggcaggccatgatcatcagttgtggcagtcaaatttgtagtggttccattgttagcagctcttgggtactcactgcggcccactgtgtcaggaatatgaatcctgaagacacagctgtaatattgggtctgaggcaccctggggcacctctgagagttgttaagatctctaccattcttctgcatgagagatttcgattggtgagtagggcagcaagaaacgatctagcattgctgctccttcaagaggtccagactcccattcagattttagcaccgctaggtcatctgaagaatctgaacagctcagaatgctggctgtctgggccacgaattcttaagccaggagagacagatgaaaatccagaaatattacagatgaaggtgataggagcttcaagctgtgcccacctttaccctgatataggcagttctattgtgtgcttcattacacaagacaaagacgctgacacaaatgtggaaccagtgagtccaggcagtgctgtcatgtgcagaccaatgtctaggaatggaagctggagacagataggcctcactagtctgaaggcactggctaccattgtgagcccccacttctcatggatattatccacttcatcaaaggcaggacatccattaaaccatgcactcatgccttggatggaaaagcctaagtcctctagtcttgtaaaacagccaaccaccctgccattttgttcaacaataattgttatactacaaaggctttcataactcattgcaaaaataaggcagggctaatctattcaaactattcataataaaaatgttaaacaatgttaaaaaaaattaagaccctatgcaacctaggaatatatgtcatgaataaac

SEQ ID NO: 14 - DNA, 6009 bp, Goat
cacagattgaaagtccagatataaacacctggagagagactgtgcctttcacagcctcacaatggacaaaggctgaacctccagctataaattcctggagagagaatatgcctttttcagtcccaccatggacacaggatggaagtccaggtgtaattacctggggagagactgtcactttcagagacccacaaaggacacaaattgaaagtccagatataaacacctggagagagactgtgcctttcatagcactaccatggacacaggctgaaggtccagatgtaaatacccagagagatactttgccttttacaggtctacgttggactcaggctgaaagtacagctgtgcatactgggagagatattgtgcctttcacagccccatcatggacacaggctgaacctccagctgtaaattcctggagagagattatgcctttttcagtcccatcatggacacaggatggaaatccaggtgtaattacctggggagaaactgtccctttcagaatcccaccaaagtcacagattgaaagtccagatgtaaacacctggagagagactatgcctttcatagcactaccatggacacaggctgaaggtccagatgtaaatacccagagagatactgtgccttttgcaggtctacactggactcaggctgaaagtacagctgtgcatactgggagagatattgtgcctttcacagccccatcatggactcaagataaaggcccagatgtaaataactggagtgagatgctgagtttcacaggaccatcatgggtacatgctaaaggtccagctttaaatacctggacagagactgtctggagggagactgtacctttaacagtctcaccttggactcagactgaaaatccaactgtaaatacctggagagagaatatgcttttaacagccctaccctggtcacaggttgaatatccagctgtaaatacatggagagaaaccgtacatttcacagagccaccatggaatcaggctgaatatcctgctgtaaacacctggagagaaactgtgcctttctcagctctacaatggactcaggctgaaagtccagctgtaaacacctggagagaggctatgcctttcacagacccatcatggattcaagataaaagtccaactgtaaatagctggagacggatttttactttcccagcccaaccatggccacagactgaaagtacagtggaaaacgacttgatatggaatgccccttttacagctccaccgtggtcacacactgaaagtccagctgtaaatacctggacagagcctatgctttttatcgcccctccatggactcaggctgaaaatccatctacaaatacctggaaagtgaatatgcattacagagacccactatggcctcagtctgactttgcaccagcaaacccttggacatcaactgaaagttttagaatcacatcatggactcttacaggaaagcaagttttaaatatttggacagagccaatagcttccacagccacactgtggactcaggctgaatattcaacaccaaaatattggacagagactaaggccatttatatagtcacatcattgacccagtgtcagtttccaataaatactttgacagaatctgtaggagccataatcacactttgtacatctgctgaatctctatcattaagttctttcacacagaatattattgatacaattgaattttggccaatggttaaaactgagtctaagaaaaggtggaatctgcctcaaactagtacattcatgttttcactaaatcctcaaactgatacttttggatccttgaaccaaattgaaaatcaagaatctcctctgtggacccatcctgaaattgataatgtcaatacaatggcgtttcttgaatttggaacactcatatcacaggtagtacctttgccccaagcagctagattctggccccaaactgaagctgatactagcaaaatttggtttgtctcctctgaaagaataaattcctgggaccaatcagagtctcaaagaatgagtacctcaatccattttggagtgggtagagtgaagcccctcgcccaacatgaaactgctacagtcatgtcatggcttcagattgaaactggtatattccacccttggaacaagtctgaaggaggcacagggaggttctggcccctttctgaaactgaggatgtaagagaatggatccaaactggagccggtacagttaactcttggactcaactgagaactaatatagtcagagcttggccccaagctgaatctgaactagtcagaccctggacacaaactaaaacgaatgcaatcacactattgacccagactgatgctatcaaaccttggttccaaactaaaattaatgcactaagagaagggacccaaactcaatctcaaattgttactacttggatccaaacacagttgcaaatatttcacccctggattcagcctaaaagtgattcagtcagattttggacccagccttggatccaagctgaaacccacacagtcagactctattatgaaattactataagaaaatcatgggcctcatctgaatctcagtcagtcacattttcatcactgagtcaaaattcagttaagaactcatttcactttgaatctcagatgacatgttcctgggtccgaaatgaatttgatataatcagtccttggaatcaatatgaaactagttctgttggatcctggttccagtctgaaactggtacctgtcaaccctggctccatattgaatcttctacaatcacaccatggacccaatatgaaacattagagatctccccttcaaccaatcctgagactgatacagcaataaggaatttgttccagccccaaattgatctaattagtacttggaatcagcctgaagtagacacaatcagattctggacccaagttgaaacagaaacaattccaatgtggacccagattggaactcaagtagttaaacctctcaacttttctgaagttggtatagttacaccttggctaaagactgaaactgatgcaagtagaccctggattcagtctgactttcagtcaatccatccgtggagccagattggatttggtataattgccccctggtctcagccaggagcttctgtaaatcaaccctggacctttgttcaaacacagtcaatcagaccttggattacagtggaatccaatagaatcaaatattggtttcatgttccaatgaaaaaagtcagactgaggattccttctgagtctcaaatattgagtttctggatgcagtctgatgttagtagagttaatgcttggatccaaccagaaacccaggcagtcaatcctggggctcatcctaaaactggcaatgttgcatccctgactattcctaaccctgaaagagtcagaatgtggatccagcctgaaacagaaataagacttggcatcatttataaaactaatatagccacatcatttgcttctgaaattgaaccagatggaacaattagttattttgattcgtggtctatccatgtaacatttttaccaatagaaactgttacttccctagatgagcattttgcagctttgtcaactgaaatagctgcagtagaaagccaaggtcaaataaattctgtccaacccagtgagatcacaaatattctctttcttacaattgcaagcacacagcttcctggaggatttggttacctgaactttggcaacaaattacaaattaccaattcaaaaggaagccctaatgtcccatatagttctctcaacccactttttccgtctttttcctttcctgttccttgttttttcccattttcatgttctttgtcccttacttgttcagtcttttcttcttgcacattttcttcaccatgtacttttccttcttgctcagttcttcctattgtgggtttctctcctgttcctcccttagctgcttctgatagttctctccagaaaccatcttcctcaaaagttattgaagacaccattctttcccatactttttcatcctttcatgctgctccagccactcttttaacaaagcaaccatctctgatgcctggatttcaattgggaaccaagtctaatcagcctgaacaagatcttcctaagtattctgaactcaatatttcccttgctgagtgtcgcctgggtgtggtctggaaagagagtctccaggctctctcgctcttcaagacagctgttatttctcatgaaatcacagagtgtggattgcgccctggccttgttccacactgtcccaactgctgggaggctgaagtgggtgaattcccttggatggtttctgtgcaactctctttctcccatttttgtgctggttctatactaaatgaacaatggattctcactacagctagatgtgcaaatttcataaaaaactcagaagcactggcccatgtccaggtggggcttatagatcttcaagaccctgctcaagctcaaactgtaggcattcatcgtgccatgccctacctgggccctagaggacctctgggacctggtctaatcttcttgaagcaaccattacattttcaacccctggttcttcctatctgcctggaggagaacctagagcaagagaaaaatatacaactgtatgactgctggctacccagttggtccctcatgagaggaagtcctggaattttgcaaaaaaggcacctgagcatcctgcaagccatcacatgtgcccagttttggcccaaactgaatgaatttactttctgtgtggcagccaagaaagctatgggggaggctggctgtaagggtgacctgggggcacctcttgtgtgtcatctgcaacaaaaagacacatgggtgcaggtgggaattttgactcactttgatgaacactgcacaaagccctacgtcttcagccaagtgagccctttccttttctggctccagggagttacacgacctagccaagcaccctggtccaagcaagggcccatgaccacctctgcttccatctccctttcagtctctacctctatgaatgcctcagcttttacttccacgcctgcttctgtccggccacatttcatctctctgccacagcctcagactttggcagatcgaatttctctgagatatgccatgccttggcaggccatgatcatcagttgtggcagtcaaatttgcagtggttccattgttagcagctcttgggtactcactgcagcccactgtgtcaggaatatgaatcctgaagacacagctgtaatattgggcctgaggcaccctggggcacctctgcgagttgttaagatctctaccattcttctgcatgaaagatttcggttggtgagtagggcagcaagaaacgatctagcattgctgctccttcaagaggtccagactcctattcagattttagcaccgctaggtcatctgaagaacctgaacagctcagaatgctggctgtctgggccacgaattcttaagccaggagagacagatgaaaatccagaaatattacagatgcaggtgataggagcatcaagctgtgcccacctttaccctgatataggtagttctattgtgtgcttcattacacaagacaaagatgctgacacaaatgtggaaccggtgagtccaggcagtgctgtcatgtgcagaccaatgtctaggaatggaagctggagacagataggcctcactagtctgaaggcactggctaccattgtgagcccccacttctcatggatattatccacttcatcaaaggcaggacatccattaagccatgcactcatgccttggatggaaaagcctaagtcctctagtatcatgaaacagccaaccactctgccattttattcaacaataattgttatactacaaaggctttaataactcattgcaaaaataatgcagggctaatctattcaaactattcataataaaaatgttaacgttaaaaaaaataaggccctat